SEQUENCE LISTING (43 sequences)

SEQ ID NO: 1 - protein, 21 aa, Human immunodeficiency virus type 1
RTWKSLVKHHMYVSGKARGWF

SEQ ID NO: 2 - protein, 14 aa, Human immunodeficiency virus type 1
WRKKRYSTQVDPEL

SEQ ID NO: 3 - protein, 2 aa, Human immunodeficiency virus type 1
FD

SEQ ID NO: 4 - protein, 2 aa, Human immunodeficiency virus type 1
CF

SEQ ID NO: 5 - protein, 9 aa, Human immunodeficiency virus type 1
DSAIRKALL

SEQ ID NO: 6 - protein, 8 aa, Human immunodeficiency virus type 1
IVSPRCEY

SEQ ID NO: 7 - protein, 11 aa, Human immunodeficiency virus type 1
QAGHNKVGSLQ

SEQ ID NO: 8 - protein, 17 aa, Human immunodeficiency virus type 1
LITPKKIKPPLPSPTKL

SEQ ID NO: 9 - protein, 11 aa, Homo sapiens
LRQESVTKQQW

SEQ ID NO: 10 - protein, 10 aa, Homo sapiens
LFSDVHAVCL

SEQ ID NO: 11 - protein, 11 aa, Homo sapiens
DKGPAKIHQAL

SEQ ID NO: 12 - protein, 36 aa, Artificial Sequence
  Chemically synthesized
LRQESVTKQQWFDLFSDVHAVCLWDDKGPAKIHQAL

SEQ ID NO: 13 - protein, 11 aa, Artificial Sequence
  Chemically synthesized
LRQESVTKQQW

SEQ ID NO: 14 - protein, 12 aa, Artificial Sequence
  Chemically synthesized
LFSDVHAVCLWD

SEQ ID NO: 15 - protein, 11 aa, Artificial Sequence
  Chemically synthesized
DKGPAKIHQAL

SEQ ID NO: 16 - protein, 23 aa, Artificial Sequence
  Chemically synthesized
LFSDVHAVCLWDDKGPAKIHQAL

SEQ ID NO: 17 - protein, 17 aa, Artificial Sequence
  Chemically synthesized
KQQWFDLFSDVHAVCLW

SEQ ID NO: 18 - protein, 12 aa, Artificial Sequence
  Chemically synthesized
KQQWFDLFSDVH

SEQ ID NO: 19 - protein, 35 aa, Artificial Sequence
  Chemically synthesized
ENLYFQSASGGHHHHGHHHHTSGGENLYFQSGGGS

SEQ ID NO: 20 - protein, 7 aa, Artificial Sequence
  Chemically synthesized
ENLYFQS

SEQ ID NO: 21 - protein, 9 aa, Artificial Sequence
  Chemically synthesized
HHHHGHHHH

SEQ ID NO: 22 - protein, 16 aa, Artificial Sequence
  Chemically synthesized
MGHHHHHHLEVLFQGP

SEQ ID NO: 23 - protein, 119 aa, Homo sapiens
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQ

SEQ ID NO: 24 - protein, 97 aa, Homo sapiens
MGYVKLISSDGHEFIVKREHALTSGTIKAMLSGPGQFAENETNEVNFREIPSHVLSKVCMYFTYKVRYTNSSTEIPEFPIAPEIALELLMAANFLDC

SEQ ID NO: 25 - protein, 102 aa, Human immunodeficiency virus type 1
SGGGSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 26 - protein, 193 aa, Human immunodeficiency virus type 1
MGENRWQVMIVWQVDRMRIRTWKSLVKHHMYVSGKARGWFYRHHYESPHPRISSEVHIPLGDARLVITTYWGLHTGERDWHLGQGVSIEWRKKRYSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 27 - protein, 197 aa, Homo sapiens
MGHHHHHHLEVLFQGPPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEVRVSQLLAVTGKKTTRP

SEQ ID NO: 28 - protein, 617 aa, Artificial Sequence
  Chemically synthesized
MGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPMHMGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFK

SEQ ID NO: 29 - protein, 390 aa, Homo sapiens
GPMHMGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFK

SEQ ID NO: 30 - DNA, 1128 bp, Artificial Sequence
  Chemically synthesized
atgggcgatgtgttcctgatgatccgccgtcacaagaccacgatttttaccgacgctaaagaatcttctaccgttttcgaactgaaacgcatcgtcgaaggtattctgaaacgcccgccggacgaacagcgtctgtataaagatgatcagctgctggatgacggcaaaaccctgggcgagtgtggtttcacttctcagacggcacgtccgcaggcccctgcaaccgttggtctggcgtttcgtgccgacgatacctttgaagctctgtgcattgaaccgttctccagcccgccagagctccccgatgtgatgaagccgcaggactctggttcttctgcgaacgaacaggcggttcagtctagaggtaagcctattcctaaccctctcctcggtctcgattctacggatatcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgaactgtacaagtag

SEQ ID NO: 31 - protein, 375 aa, Artificial Sequence
  Chemically synthesized
MGDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQSRGKPIPNPLLGLDSTDIVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 32 - DNA, 1317 bp, Artificial Sequence
  Chemically synthesized
atgggacatcaccatcaccatcacctggaagttctgttccaggggcccccgcgcgtcgtgcccgaccagagaagcaagttcgagaacgaggagttttttaggaagctgagccgcgagtgtgagattaagtacacgggcttcagggaccggccccacgaggaacgccaggcacgcttccagaacgcctgccgcgacggccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggtgagagtttcacagctgctggcagtaactggcaagaagacaacaagacccggagaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgaactgtacaagtag

SEQ ID NO: 33 - protein, 438 aa, Artificial Sequence
  Chemically synthesized
MGHHHHHHLEVLFQGPPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEVRVSQLLAVTGKKTTRPGEFVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 34 - DNA, 1305 bp, Artificial Sequence
  Chemically synthesized
atgggagaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatctgggtcagggagtctccatagaatggaggaaaaagagatatagcacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacacggagaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgaactgtacaagtag

SEQ ID NO: 35 - protein, 434 aa, Artificial Sequence
  Chemically synthesized
MGENRWQVMIVWQVDRMRIRTWKSLVKHHMYVSGKARGWFYRHHYESPHPRISSEVHIPLGDARLVITTYWGLHTGERDWHLGQGVSIEWRKKRYSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGHGEFVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 36 - DNA, 2613 bp, Artificial Sequence
  Chemically synthesized
atgggctcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgcatatgggaggatccacgtctaatctgttaaagaataaaggttctcttcagtttgaagacaaatgggattttatgcgcccgattgttttgaagcttttacgccaggaatctgttacaaaacagcagtggtttgatctgttttcggatgtgcatgcagtctgtctttgggatgataaaggcccagcaaaaattcatcaggctttaaaagaagatattcttgagtttattaagcaggcacaggcacgagtactgagccatcaagatgatacggctttgctaaaagcatatattgttgaatggcgaaagttctttacacaatgtgatattttaccaaaacctttttgtcaactagagattactttaatgggtaaacagggcagcaataaaaaatcaaatgtggaagacagtattgttcgaaagcttatgcttgatacatggaatgagtcaatcttttcaaacataaaaaacagactccaagatagtgcaatgaagctggtacatgctgagagattgggagaagcttttgattctcagctggttattggagtaagagaatcctatgttaacctttgttctaatcctgaggataaacttcaaatttatagggacaattttgagaaggcatacttggattcaacagagagattttatagaacacaagcaccctcgtatttacaacaaaatggtgtacagaattatatgaaatatgcagatgctaaattaaaagaagaagaaaaacgagcactacgttatttagaaacaagacgagaatgtaactccgttgaagcactcatggaatgctgtgtaaatgccctggtgacatcatttaaagagactatcttagctgagtgccaaggcatgatcaagagaaatgaaactgaaaaattacatttaatgttttcattgatggacaaagttcctaatggtatagagccaatgttgaaagacttggaggaacatatcattagtgctggcctggcagatatggtagcagctgctgaaactattactactgactctgagaaataccgtgagcagttagatacactatttaatagatttagtaaactcgtcaaagaagcttttcaagatgatccacgatttcttactgcaagagataaggcgtataaagcagttgttaatgatgctaccatatttaaatctagaggcggttacccctacgacgtgcccgactacgccgatatcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttaatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactggaacagcgtcaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagcgcgtgaccgccgccgggatcactctcggcatggacgagctgtacaagtag

SEQ ID NO: 37 - protein, 870 aa, Artificial Sequence
  Chemically synthesized
MGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLHMGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFKSRGGYPYDVPDYADIVSKGEELFTGVVPILVELDGDVNGHKFSVSGEGEGDATYGKLTLKLICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNWNSVNVYIMADKQKNGIKVNFKIRHNIEDGSVQLADHYQQNTPIGDGPVLLPDNHYLSYQSALSKDPNEKRDHMVLLERVTAAGITLGMDELYK

SEQ ID NO: 38 - DNA, 1938 bp, Artificial Sequence
  Chemically synthesized
atgggacatcaccatcaccatcacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttaatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactggaacagcgtcaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagcgcgtgaccgccgccgggatcactctcggcatggacgagctgtacaagcaggaattcctggaagttctgttccaggggcccggtggtagtggtggatccacgtctaatctgttaaagaataaaggttctcttcagtttgaagacaaatgggattttatgcgcccgattgttttgaagcttttacgccaggaatctgttacaaaacagcagtggtttgatctgttttcggatgtgcatgcagtctgtctttgggatgataaaggcccagcaaaaattcatcaggctttaaaagaagatattcttgagtttattaagcaggcacaggcacgagtactgagccatcaagatgatacggctttgctaaaagcatatattgttgaatggcgaaagttctttacacaatgtgatattttaccaaaacctttttgtcaactagagattactttaatgggtaaacagggcagcaataaaaaatcaaatgtggaagacagtattgttcgaaagcttatgcttgatacatggaatgagtcaatcttttcaaacataaaaaacagactccaagatagtgcaatgaagctggtacatgctgagagattgggagaagcttttgattctcagctggttattggagtaagagaatcctatgttaacctttgttctaatcctgaggataaacttcaaatttatagggacaattttgagaaggcatacttggattcaacagagagattttatagaacacaagcaccctcgtatttacaacaaaatggtgtacagaattatatgaaatatgcagatgctaaattaaaagaagaagaaaaacgagcactacgttatttagaaacaagacgagaatgtaactccgttgaagcactcatggaatgctgtgtaaatgccctggtgacatcatttaaagagactatcttagctgagtgccaaggcatgatcaagagaaatgaaactgaaaaattacatttaatgttttcattgatggacaaagttcctaatggtatagagccaatgttgaaagacttggaggaacatatcattagtgctggcctggcagatatggtagcagctgctgaaactattactactgactctgagaaataccgtgagcagttagatacactatttaatagatttagtaaactcgtcaaagaagcttttcaagatgatccacgatttcttactgcaagagataaggcgtataaagcagttgttaatgatgctaccatatttaaataa

SEQ ID NO: 39 - protein, 645 aa, Artificial Sequence
  Chemically synthesized
MGHHHHHHVSKGEELFTGVVPILVELDGDVNGHKFSVSGEGEGDATYGKLTLKLICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNWNSVNVYIMADKQKNGIKVNFKIRHNIEDGSVQLADHYQQNTPIGDGPVLLPDNHYLSYQSALSKDPNEKRDHMVLLERVTAAGITLGMDELYKQEFLEVLFQGPGGSGGSTSNLLKNKGSLQFEDKWDFMRPIVLKLLRQESVTKQQWFDLFSDVHAVCLWDDKGPAKIHQALKEDILEFIKQAQARVLSHQDDTALLKAYIVEWRKFFTQCDILPKPFCQLEITLMGKQGSNKKSNVEDSIVRKLMLDTWNESIFSNIKNRLQDSAMKLVHAERLGEAFDSQLVIGVRESYVNLCSNPEDKLQIYRDNFEKAYLDSTERFYRTQAPSYLQQNGVQNYMKYADAKLKEEEKRALRYLETRRECNSVEALMECCVNALVTSFKETILAECQGMIKRNETEKLHLMFSLMDKVPNGIEPMLKDLEEHIISAGLADMVAAAETITTDSEKYREQLDTLFNRFSKLVKEAFQDDPRFLTARDKAYKAVVNDATIFK

SEQ ID NO: 40 - DNA, 1506 bp, Artificial Sequence
  Chemically synthesized
atgggacatcaccatcaccatcacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttaatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactggaacagcgtcaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagcgcgtgaccgccgccgggatcactctcggcatggacgagctgtacaagcaggaattcctggaagttctgttccaggggcccggtggtagtggtggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgaactgtacaagtag

SEQ ID NO: 41 - protein, 501 aa, Artificial Sequence
  Chemically synthesized
MGHHHHHHVSKGEELFTGVVPILVELDGDVNGHKFSVSGEGEGDATYGKLTLKLICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNWNSVNVYIMADKQKNGIKVNFKIRHNIEDGSVQLADHYQQNTPIGDGPVLLPDNHYLSYQSALSKDPNEKRDHMVLLERVTAAGITLGMDELYKQEFLEVLFQGPGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 42 - DNA, 42 bp, Artificial Sequence
  Chemically synthesized
ctggaagttctgttccaggggcccggtggtagtggtggatcc

SEQ ID NO: 43 - protein, 14 aa, Artificial Sequence
  Chemically synthesized
LEVLFQGPGGSGGS

(19) United States
(12) Patent Application Publication
(10) Pub. No.: US 2015/0268247 A1
(43) Pub. Date: Sep. 24, 2015

(54) Assay for Screening of Anti-Viral Compounds That Inhibit Specific Interaction Interfaces Between Cullin5 and an ElonginB/ElonginC/ CBF-beta/HIV-1 Vif Complex

(71) Applicant: UNIVERSITY OF ROCHESTER, Rochester, NY (US)

(72) Inventors: Joseph E. Wedekind, Rochester, NY (US); Jason D. Salter, Rochester, NY (US); Geoffrey M. Lippa, East Rochester, NY (US); Ivan A. Belashov, Rochester, NY (US)

(21) Appl. No.: 14/435,860

(22) PCT Filed: Oct. 22, 2013
(86) PCT No.: PCT/US13/66170

§ 371 Date: Apr. 15, 2015

Related U.S. Application Data
(60) Provisional application No. 61/716,916, filed on Oct. 22, 2012.

(51) Int. Cl. G01N 33/68; C07K 14/47; C07K 14/005; G01N 21/64

(52) U.S. Cl. CPC G01N 33/6845; G01N 21/6428; C07K 14/47; C07K 14/005; C12N 2740/16322; G01N 2021/6432; G01N 2333/4703; G01N 2333/163; A61K 38/00

(57) ABSTRACT

The present invention relates to the production of an ElonginB/ElonginC/Vif/CBFβ tetramer complex comprising full-length Vif protein. The present invention provides an assay for screening any agent that inhibits the ability of Vif to bind with Cul5. The invention provides an agent identified by the screening methods and methods of treatment using the identified agent. The invention also provides compositions that inhibit Vif-Cul5 binding based upon regions identified in Vif and Cul5 that mediate Vif-Cul5 binding.

### CROSS-REFERENCE TO RELATED APPLICATIONS

This application claims priority to U.S. Provisional Application Ser. No. 61/716,916, filed Oct. 22, 2012, the contents of which are incorporated by reference herein in their entirety.

### STATEMENT REGARDING FEDERALLY SPONSORED RESEARCH OR DEVELOPMENT

This invention was made with government support under NIH R33 AI076085 awarded by National Institutes of Health. The government has certain rights in the invention.

### BACKGROUND OF THE INVENTION

Viral infections can be accompanied by the hijacking of cellular pathways to subvert innate defense mechanisms (Barry and Fruh, 2006, Sci STKE 2006(335):pe21). This is exemplified by HIV-1 in which an essential protein, viral infectivity factor (Vif), neutralizes APOBEC3G (A3G) and related family members inherent to CD4(+) T cells [reviewed in (Wolf and Goff, 2008, Annu Rev Genet 42:143-163)]. In Vif deficient HIV-1 infection, A3G incorporates into virions and travels to subsequently infected cells where it exhibits antiviral properties including dC-to-dU deamination of first-strand HIV-1 DNA (Harris et al., 2003, Cell 113:803-809; Mangeat et al., 2003, Nature 424:99-103). In wild-type HIV-1 infections, however, Vif masquerades as a SOCS-box substrate receptor that directly binds A3G via conserved sequences [reviewed in (Dang et al., 2010, J Virol 84:8561-8570) and FIG. 1], and recruits it to a Cullin-RING E3 ubiquitin ligase resulting in polyubiquitination and proteasomal degradation (FIG. 1A) (Marin et al., 2003, Nat Med 9:1398-1403; Sheehy, et al., 2003, Nat Med 9:1404-1407). Vif binds EloC via a canonical BC-box conserved in cellular SOCS-box proteins (Stanley et al., 2008, J Virol 82:8656-8663; Yu et al., 2004, Genes Dev 18:2867-2872) but utilizes a novel HCCH Zn²⁺-binding motif to associate with N-terminal Cul5 regions in lieu of the cellular Cul5-box (Xiao et al., 2006, Virology 349:290-299; Wolfe et al., 2010, J Virol 84:7135-7139). The model for E3 complex formation posits that the SOCS-box/EloB/C interaction precedes Cul5 binding (Babon et al., 2009, J Mol Biol 387:162-174).

Thus, the formation of a Vif-comprising protein complex, and the eventual binding to Cullin5, results in degradation of the host anti-viral protein A3G and related family members, thereby permitting viral infectivity. While such protein interactions have been known for some time, the development of anti-viral therapeutics based upon the inhibition of these protein-protein interactions have been lacking One reason for the dearth of inhibitors is due to the absence of an effective screening system that allows for observing the effects of potential compounds on the activity of full-length Vif protein. Vif protein has been notoriously difficult to produce recombinantly, which has hampered progress in developing effective screens.

Thus, there is a need in the art for the development of effective screening methods and the identification of compounds that inhibit the Vif-mediated degradation of A3G and other antiretroviral family members. The present invention satisfies this unmet need.

### SUMMARY OF THE INVENTION

The present invention provides a method of producing soluble, functionally active, full-length HIV-1 Vif. The method comprises providing to a cell exogenous ElonginB, ElonginC, Vif, and CBFβ polynucleotide; expressing exogenous ElonginB, ElonginC, Vif, and CBFβ polypeptide; and isolating an ElonginB/ElonginC/Vif/CBFβ tetramer complex from the cell.

The present invention also includes an isolated protein complex comprising ElonginB, ElonginC, full-length Vif, and CBFβ, where full-length Vif is able to bind Cullin5 at benchmarked levels. In one embodiment, the isolated protein complex further comprises Cullin5. In one embodiment, the isolated protein complex comprises amino acids 1-118 of said ElonginB, and amino acids 17-112 of ElonginC.

The present invention also includes a method of identifying a compound that inhibits the interaction between Vif and Cullin5. The method comprises providing a mixture comprising Cullin5, a protein complex comprising ElonginB, ElonginC, Vif, and CBFβ, and a test compound under conditions that are effective for binding of Vif to Cullin5; and detecting whether or not the test compound inhibits binding of Vif to Cullin5, thereby identifying a compound that inhibits the interaction between Vif and Cullin5.

In one embodiment, the test compound that inhibits the binding between Vif and Cullin5 is an inhibitor of lentiviral infectivity.

In one embodiment, the method is a high throughput method. In one embodiment the high throughput method is Förster quenched resonance energy transfer (FqRET). In one embodiment, at least one of ElonginB, ElonginC, Vif, and CBFβ is labeled with a FRET donor and Cullin5 is labeled with a FRET quencher. In one embodiment, detecting whether or not the test compound inhibits binding of Vif to Cullin5 comprises detecting an increase in fluorescence compared to a condition where the test compound is absent.

In one embodiment, the mixture comprises Brij 35 and glycerol.

In one embodiment, the mixture is formed by providing a first mixture comprising the protein complex and Cullin5, and contacting the first mixture with the test compound. In one embodiment, the mixture is formed by providing a first mixture comprising the protein complex, contacting the first mixture with the test compound to produce a second mixture, and contacting the second mixture with Cullin5. In one embodiment, the mixture is formed by providing a first mixture comprising Cullin5, contacting the first mixture with the test compound to produce a second mixture, and contacting the second mixture with the protein complex.

The present invention also includes a composition that inhibits the binding of full-length Vif to Cullin5. In one embodiment, the composition is identified by a screening method comprising the steps of providing a mixture comprising Cullin5, a protein complex comprising ElonginB, ElonginC, Vif, and CBFβ, and a test compound under conditions that are effective for binding of Vif to Cullin5; and detecting whether or not the test compound inhibits binding of Vif to Cullin5.

In one embodiment, the composition comprises at least one amino acid sequence selected from the group consisting of SEQ ID NOs: 1-18. In one embodiment the composition binds to an epitope of Vif, where the epitope is defined by at least one amino acid sequence selected from the group consisting of SEQ ID NOs: 1-8. In one embodiment, the composition binds to an epitope of Cullin5, where the epitope is defined by at least one amino acid sequence selected from the group consisting of SEQ ID NOs: 9-11.

In one embodiment, the composition reduces the affinity for a Vif-Cullin5 interaction by inhibiting the binding of Vif and CBFβ.

The present invention also includes a method for inhibiting infectivity of a lentivirus. The method comprises contacting a cell that is producing the virus with an antiviral-effective amount of a composition that inhibits Vif-Cullin5 binding. In one embodiment, the antiviral-effective amount of the composition does not substantially affect proteins in the cell other than lentivirus Vif.

In one embodiment, the composition is identified by a screening method comprising the steps of providing a mixture comprising Cullin5, a protein complex comprising ElonginB, ElonginC, Vif, and CBFβ, and a test compound under conditions that are effective for binding of Vif to Cullin5; and detecting whether or not the test compound inhibits binding of Vif to Cullin5.

In one embodiment, the lentivirus expresses Vif. In one embodiment, the lentivirus is HIV.

In one embodiment, the composition inhibits the interaction of Vif with cellular Cullin5-E3 ubiquitin ligase, thereby preventing the degradation of the viral inhibitor, APOBEC3G and/or related family members, and thus allowing the APOBEC3G and/or related family members to inhibit viral infectivity.

In one embodiment, the composition comprises at least one amino acid sequence selected from the group consisting of SEQ ID NOs: 1-18. In one embodiment the composition binds to an epitope of Vif, where the epitope is defined by at least one amino acid sequence selected from the group consisting of SEQ ID NOs: 1-8. In one embodiment, the composition binds to an epitope of Cullin5, where the epitope is defined by at least one amino acid sequence selected from the group consisting of SEQ ID NOs: 9-11.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawing.

FIG. 1, comprising FIG. 1A and FIG. 1B, is a schematic of the Vif-mediated E3 ligase and Vif sequence motifs. FIG. 1A illustrates that A3G is recruited by Vif to the N-terminus of Cullin 5 (herein called Cul5(N)) in conjunction with the heterodimeric EloB/C substrate adaptor. Cul5(C) and Rbx2 position the E2 ubiquitin conjugase. FIG. 1B depicts conserved Vif binding motifs.

FIG. 2 is a graph depicting the heat capacity change (ΔCp) for interaction of Cul5(N) with Vif_{C}/EloB/C and Vif/EloB/C/CBFβ taken as the slope of best-fit lines. Here, Vif_{C} is truncated Vif (amino acids 95-192), while Vif in the context of the Vif/EloB/C/CBFβ is full-length Vif (amino acids 1-192).

FIG. 3, is a set of images depicting Coomassie-stained SDS-PAGE analysis of Vif_{C}/EloB/C, Vif/EloB/C/CBFβ, SOCS2_{SOCS-box}/EloB/C, and Cul5(N). Purified protein complexes and Cul5(N) are >95% pure. Vif_{C}/EloB/C and Cul5(N) samples was electrophoresed in NuPAGE 4-12% Bis-Tris gels (Invitrogen) while Vif/EloB/C/CBFβ and SOCS2_{SOCS-box}/EloB/C were electrophoresed in MiniProtean TGX AnyKD gels (BioRad). Protein bands were confirmed with peptide mass mapping (not shown) at the PAN Facility (Stanford University).

FIG. 4, comprising FIG. 4A through FIG. 4F, is a set of Representative ITC isotherms at 303.15 K in ITC Buffer A for the titration of Cul5(N) to: (FIG. 4A) Vif_{C}/EloB/C, (FIG. 4B) Vif/EloB/C/CBFβ, (FIG. 4C) SOCS2_{SOCS-box}/EloB/C, and (FIG. 4D) CBFβ alone. Other controls included: (FIG. 4E) Titration of CBFβ into Vif_{C}/EloB/C and (FIG. 4F) titration of Cul5(N) to ITC buffer A as a negative control.

FIG. 5, comprising FIG. 5A and FIG. 5B, is a set of representative ITC isotherms for measurement of temperature-dependent enthalpies of Cul5(N) binding to (FIG. 5A) Vif_{C}/EloB/C, and (FIG. 5B) Vif/EloB/C/CBFβ.

FIG. 6, comprising FIG. 6A and FIG. 6B, are a set of illustrations depicting the Sequence alignments of cellular SOCS-box type E3 ubiquitin ligase substrate receptors and the HIV-1 Vif serotypes. FIG. 6A illustrates the sequence alignment of SOCS-box motif from cellular SOCS-box type E3 ubiquitin ligase substrate receptors. The SOCS-box motif of cellular SOCS-box type substrate receptors (Receptor_{SOCS-box}) comprises: i) the BC-box motif that mediates binding to EloC of the EloB/C heterodimer, and ii) the Cul5-box that is necessary for Cul5 binding by cellular receptor_{SOCS-box}/EloB/C complexes (reviewed in Mahrour et al., 2008, J Biol Chem, 283: 8005-8013). FIG. 6B depicts the sequence alignment of HIV-1 Vif Cul5- and EloC-binding motifs. Primary sequence that depicts how HIV-1 Vif can mimic the cellular Receptor_{SOCS-box} based on a completely conserved BC-box motif, but only a partial Cul5-box with poor conservation (Yu et al., 2003, Science, 302: 1056-1060; Yu et al., 2004, Genes Dev, 18: 2867-2872). Consequently, Vif utilizes the HCCH Zn²⁺-binding motif to bind Cul5 (Luo et al., 2005, Proc Natl Acad Sci USA, 102: 11444-11449; Mehle et al., 2006, Proc Natl Acad Sci USA, 103: 18475-18480; Wolfe et al., 2010, J Virol, 84: 7135-7139; Xiao et al., 2006, Virology, 349: 290-299). Conserved hydrophobic residues within the motif have been implicated in the Cul5 interaction (Xiao et al., 2006, Virology, 349: 290-299; Xiao et al, 2007, FASEB J, 21: 217-222).

FIG. 7 is an image depicting the expression of purified Cul5(N) (lane 1), EloB/C/Vif/CBFβ tetramer (lane 2), and EloB/C/Vif/CBFβ/Cul5(N) (pentamer) for HDX-MS.

FIG. 8 is an image depicting the deuteration perturbation levels for EloB, EloC, Vif, CBFB, and Cul5. Perturbation levels for peptic fragments of each protein are indicated by the bars for 15, 50, 150, 500, 1500, 5000 s. Protein sequences are provided for EloB, EloC, Vif, CBFβ, and Cul5. Perturbation is equal to the difference in deuteration levels for the Bound (pentameric) and Unbound (respective tetrameric complex and Cul5(N) alone) complexes. A negative perturbation level is indicative of protection from deuteration upon pentameric complex formation. A positive perturbation is indicative of an acceleration of deuteration upon formation of the pentameric complex.

FIG. 9 is a schematic of peptide sequences (SEQ ID NOs: 12-18).

FIG. 10 is a schematic depicting the structural mapping of peptides to a crystal structure of EloB/C/SOCS2/Cul5(N) demonstrating that region B forms an interface with EloC, but most of region A and region C are exposed.

FIG. 11 is a set of graphs depicting the results of fluorescence anisotropy assays investigating the binding affinity of the peptides for the Vif/EloB/EloC/CBFβ complex or control.

FIG. 12, comprising FIG. 12A through FIG. 12D, is a set of representative ITC isotherms at 303.15 K for the titration of Cul5(N) to: (FIG. 12A) Vif/EloB/C/CBFβ, (FIG. 12B) Vif_{C}/EloB/C, (FIG. 12C) EloB/C, and (FIG. 12D) CBFβ to Vif_{C}/EloB/C.

FIG. 13 is an image depicting the purification of Vif/EloB/C/CBFβ complexes, where one of the proteins is tagged with mEGFP, and Cul5(N), which is tagged with sREACh either on its N-terminal or C-terminal end.

FIG. 14 is a table summarizing the binding of various tagged quaternary complexes with tagged Cul5(N) demonstrating that the presence of a FRET tag does not negatively influence the binding of Cul5(N) to the complex.

FIG. 15 is a set of representative ITC isotherms for the titration of Cul5(N)-sREACh to Vif/EloB-mEGFP/EloC/CBFβ (left), Vif/EloB/EloC/CBFβ-mEGFP (center), or Vif-mEGFP/EloB/EloC/CBFβ (right).

FIG. 16 is a graph depicting the quenching of Vif/EloB/EloC/CBFβ-mEGFP at 508 nm, when excited at 469 nm, upon addition of various concentrations of Cul5(N)-sREACh.

FIG. 17 is a graph depicting the loss of protein fluorescence at room temperature of mEGFP tagged quaternary complexes (10 nM) with addition of either 4-fold molar excess of Cul5(N), Cul5(N)-sREACh (quencher), or both. Proteins were incubated at room temperature for up to 20 hours, where fluorescence intensity was monitored at various time points shown.

FIG. 18 is a set of graphs depicting the fluorescence intensity at 4° C. (left) and 25° C. (right) of 10 nm Vif/EloB/EloC/CBFβ-mEGFP incubated in a solution containing the identified agents, used to determine if such agents can inhibit or reduce the loss in fluorescence over time.

FIG. 19 depicts a set of graphs depicting the comparison between the loss of fluorescence over time in non-detergent-containing assay proteins (left) to those with various amounts of Brij 35 (right). The Vif/B/C/CBFβ-mEGFP complex (box) is shown (right) in the presence of varying concentrations of Brij 35.

FIG. 20 depicts the quenching between quaternary complexes and Cul5(N)-sREACh. Quenching was observed over time for Cul5(N)-sREACh binding to Vif/EloB/EloC/CBFβ-mEGFP (left), Vif/EloB-mEGFP/EloC/CBFβ (center), and Vif-mEGFP/EloB/EloC/CBFβ (right). Quenching was observed with 10 μM quaternary complex in addition to 10 μM, 40 μM, or 80 μM Cul5(N)-sREACh.

FIG. 21 is a graph (left) depicting the increase in fluorescence (loss of FRET quenching) of a positive control fusion protein comprising mEGFP and sREACh separated by a short flexible protease-cleavable linker. The Coomasie-stained electrophoresis gel (right) demonstrates the cleavage of the fusion protein over time.

FIG. 22 depicts the quenching between quaternary complexes and sREACh-Cul5(N), where sREACh and Cul5(N) are separated by the linker used in the positive control fusion protein (FIG. 21). Quenching was observed for sREACh-Cul5(N) binding to Vif/EloB-mEGFP/EloC/CBFβ (left), Vif/EloB/EloC/CBFβ-mEGFP (center), and Vif-mEGFP/EloB/EloC/CBFβ (right).

### DETAILED DESCRIPTION OF THE INVENTION

The present invention is based, at least in part, on the discovery that expression of a recombinant complex comprising full-length Vif (residues 1-192) is predicated upon the inclusion of core-binding factor B (CBFβ). In one aspect, the present invention provides a screening assay comprising a recombinant complex of ElonginB/ElonginC/Vif/CBFβ and recombinant Cullin5 (Cul5). Preferably, the recombinant complex and recombinant Cullin5 protein are soluble. The assay is useful in identifying agents that are able to inhibit the interaction between Vif and Cul5. In one embodiment, the assay of the invention is able to screen for an agent that inhibits the ability of Vif to bind with Cul5. Without wishing to be bound by any particular theory, inhibiting Vif binding to Cul5 also inhibits recruitment of anti-viral host factors to the cellular E3 ligase, which is thought to be an important component of the cellular ubiquitin protease machinery that degrades the host's innate immune proteins, such as those of the APOBEC3 family of DNA deaminases. The agent can target a domain in the Vif protein that is required for the interaction with Cul5. In some instances, the agent can target a domain in the Cul5 protein that is required for the interaction with Vif. In another instance, the agent can target both a domain in the Vif protein and a domain in the Cul5 protein. In another instance, the agent can target other host proteins including CBFβ, ElonginB, or ElonginC.

The present invention provides a full-length Vif-mediated assay and agents identified by the Vif-mediated assay. Accordingly, the invention provides a method of preventing ubiquitination of APOBEG3G (A3G) and related antiretroviral family members that act on HIV-1, without broadly inhibiting the cell's ability to carry out ubiquitination on other proteins.

Inhibiting or reducing the interaction between Vif and Cul5 allows the virus to become sensitive to the antiviral activities of A3G (or one of the other noted APOBEC3 proteins). Therefore, if a cell that is producing virus is treated with an agent that inhibits Vif and Cul5 interaction, virus that is being produced by the cell is inactivated and thus is unable (or exhibits a reduced capacity) to carry out future rounds of infection. In this manner, infectivity of the virus is inhibited by the compounds identified by the screening methods of the invention.

The present invention provides a screening method comprising the inclusion of CBFβ into the full-length Vif-comprising protein complex. As described elsewhere herein, inclusion of CBFβ is shown to have certain benefits compared to a system that does not comprise CBFβ. The method disclosed herein allows for rapid screening of agents for their ability to inhibit interaction between Vif and Cul5, which yields agents that are important potential therapeutics for use in methods where selective inhibition of Vif and Cul5 binding provides a therapeutic benefit, including, but not limited to, development of agents useful for treating viral infection, while reducing the risk of cell toxicity that might otherwise arise form general inhibitors of ubiquitination. Preferably, the viral infection is HIV-1.

In one aspect, the invention provides a composition that inhibits the binding of Vif and Cullin5. The composition of the invention can therefore reduce ubiquitination of APOBEC3G, or related family members, and reduce viral infectivity in a subject. The present invention is partly based upon the identification of sequences within Vif and Cullin5 that are critical for the formation of the ElonginB/ElonginC/Vif/CBFβ/Cullin5 complex that eventually results in degradation of APOBEC3G or related family members. Inclusion of CBFβ within the Vif complex allows for expression of full-length Vif, and therefore allows analysis of the complete Vif sequence for regions integral in Cul5 binding. Such an analysis has, until now, been impossible. Further, inclusion of CBFβ allows for the binding of full-length Vif with 5 nm avidity to Cul5(N). Such an avid interaction has not been demonstrated until now, and the analysis of this system has not been impossible. In one embodiment, the composition is a peptide inhibitor comprising at least one of the identified sequences. In one embodiment, the peptide inhibitor comprises at least one sequence selected from the group consisting of SEQ ID NOs: 1-18. In one embodiment, the composition is a peptide inhibitor that competes with Vif for binding to Cullin5. In one embodiment, the composition is a peptide inhibitor that competes with Cullin5 for binding to Vif. In another embodiment, the composition directly or indirectly binds to an epitope of Vif defined by at least one of the identified sequences, thereby inhibiting the ability of Vif to bind to Cullin5. In one embodiment, the composition binds to an epitope defined by at least one of SEQ ID NOs: 1-8 of Vif protein. In another embodiment, the composition directly or indirectly binds to an epitope of Cullin5 defined by at least one of the identified sequences, thereby inhibiting the ability of Cullin5 to bind to Vif. In one embodiment, the composition is an antibody, or fragment thereof, that binds to an epitope defined by at least one of SEQ ID NOs: 9-11 of Cullin5 protein.

In one embodiment, the present invention is directed to a composition that inhibits the binding of Vif and CBFβ. As detailed elsewhere herein, it is found that the affinity of Vif-Cul5 binding is drastically increased when Vif is complexed with CBFβ. Thus, in one embodiment, the composition of the invention prevents Vif binding with CBFβ, and therefore reduces the affinity of Vif-Cul5 binding. In one embodiment, the composition that inhibits Vif-CBFβ binding reduces degradation of APOBEC3G (or other APOBEC3 proteins), thereby reducing viral infectivity.

### DEFINITIONS

As used herein, each of the following terms has the meaning associated with it in this section.

The articles “a” and “an” are used herein to refer to one or to more than one (e.g., to at least one) of the grammatical object of the article. By way of example, “an element” means one element or more than one element.

The term “binding” refers to a direct association between at least two molecules, due to, for example, covalent, electrostatic, hydrophobic, ionic and/or hydrogen-bond interactions under physiological conditions.

A “fusion protein” is a fusion of a first amino acid sequence encoded by a polynucleotide with a second amino acid sequence defining a domain foreign to and not substantially homologous with any domain of the first amino acid sequence.

As used herein, the term “fragment,” as applied to a nucleic acid, refers to a subsequence of a larger nucleic acid. A “fragment” of a nucleic acid can be at least about 20 nucleotides in length; for example, at least about 50 nucleotides to about 100 nucleotides; preferably at least about 100 to about 500 nucleotides, more preferably at least about 500 to about 1000 nucleotides, even more preferably at least about 1000 nucleotides to about 1500 nucleotides; particularly, preferably at least about 1500 nucleotides to about 2500 nucleotides; most preferably at least about 2500 nucleotides.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, lentiviruses, adenoviruses, and adeno-associated viruses) that incorporate the recombinant polynucleotide.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic mammals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operatively linked by sequential attachment to the adjacent element. A gene comprising the above elements is inserted by standard recombinant DNA methods into any expression vector.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 5′TATGGC3′ share 50% homology.

As used herein, “homology” is used synonymously with “identity.”

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

As used herein, an “inhibitory-effective amount” is an amount that results in a detectable (e.g., measurable) amount of inhibition of an activity of Vif, such as its ability to target and degrade A3G in a cell infected by a lentivirus. In some instance, the activity of Vif is its ability to bind with Cullin5.

The term “lentivirus” as used herein may be any of a variety of members of this genus of viruses. In one embodiment of the invention, the lentivirus contains a Vif gene. The lentivirus may be, e.g., one that infects a mammal, such as a sheep, goat, horse, cow or primate, including human. Typical such viruses include, e.g., Vizna virus (which infects sheep); simian immunodeficiency virus (SIV), bovine immunodeficiency virus (BIV), chimeric simian/human immunodeficiency virus (SHIV), feline immunodeficiency virus (FIV) and human immunodeficiency virus (HIV). “HIV,” as used herein, refers to both HIV-1 and HIV-2. Much of the discussion herein is directed to HIV or HIV-1; however, it is to be understood that other suitable lentiviruses are also included.

The term “mammal” as used herein refers to any non-human mammal. Such mammals are, for example, rodents, non-human primates, sheep, dogs, cows, and pigs. The preferred non-human mammals are selected from the rodent family including rat and mouse, more preferably mouse. The preferred mammal is a human.

A “nucleic acid molecule” is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications. In addition, “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. Essentially, the pharmaceutically acceptable material is nontoxic to the recipient. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

As used herein, “pharmaceutical compositions” include formulations for human and veterinary use.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, transformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from,” with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. In certain instances the term “antiviral-effective amount” refers to the amount of the subject compound that prevents, reduces, inhibits, delays, or slows viral activity to some extent.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

“Viral infectivity” as that term is used herein means any of the infection of a cell, the replication of a virus therein, and the production of progeny virions therefrom.

A “virion” is a complete viral particle; nucleic acid and capsid, further including and a lipid envelope in the case of some viruses.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is partly based on the discovery that recombinant full-length Vif is efficiently produced when co-expressed with CBFβ. Further, data presented herein provides quantitative validation of this interaction thereby allowing the delineation of specific chemical properties of the Vif-Cul5 interaction that contribute to the measured high-affinity of the interaction. The production of full-length Vif, in the context of an ElonginB/ElonginC/Vif/CBFβ tetramer complex allows for high throughput screening of Vif-Cul5 interactions. Without wishing to be bound by any particular theory, the present method of assessing Vif-Cul5 interactions allows for assessing the activity of the Cullin-RING E3 ligase that is responsible for degradation of the innate antiviral proteins such as APOBEC3G (A3G), APOBEC3F (A3F), and related APOBEC3 family members.

The unique design of the recombinant proteins enables the ability to generate ElonginB/ElonginC/Vif/CBFβ as well as Cullin5 in soluble forms in a large-scale production setting, producing milligram or greater levels of pure protein, which allows for the development of an assay to screen for agents that block the Vif interaction with Cullin5. The assay provides a method to screen for any agent that inhibits the ability of Vif to bind with Cul5. The agent can target a domain in the Vif protein, Cul5 protein, or both whereby interaction between Vif and Cul5 is inhibited.

The invention is also partly based upon the identification of sequences within Vif and Cul5 that play a role in Vif-Cul5 binding. Such sequences were identified by analyzing the Vif-Cul5 interface within the ElonginB/ElonginC/Vif/CBFβ/Cul5 pentameric complex. The present invention is directed to a composition that prevents or inhibits Vif-Cul5 binding. In one embodiment, the composition comprises at least one of the identified sequences and competes with Vif and/or Cul5, thereby inhibiting Vif-Cul5 binding. In another embodiment, the composition binds to an epitope of Vif and/or Cul5 defined by at least one of the identified sequences, thereby inhibiting Vif-Cul5 binding. In another embodiment, the composition is identified by a high-throughput screen for its ability to reduce Vif-Cul5 binding. In another embodiment, the composition inhibits Vif-CBFβ binding, thereby reducing the affinity for Vif-Cul5 binding.

### Screening Compositions

In one embodiment, the present invention is directed to a composition that is useful in a screening assay for identifying agents that inhibit Vif-Cul5 binding. The invention is partly based upon the discovery that full-length Vif expression and function is greatly enhanced by the co-expression of CBFβ. In one embodiment, the composition comprises a peptide, or derivative thereof, wherein the peptide comprises at least one of ElonginB, ElonginC, Vif, CBFβ, Cul5, or fragments thereof. In one embodiment, the composition comprises a nucleic acid sequence encoding at least one of ElonginB, ElonginC, Vif, CBFβ, Cul5, or fragments thereof. In one embodiment, the composition comprises a detectable signal for use in a screening assay of the invention.

In one embodiment, the compositions of the invention allow for the large scale production of ElonginB/ElonginC/Vif/CBFβ tetramer complex and Cul5 protein. The present invention is at least partly based upon the validated high-affinity and homogenous composition of the produced proteins. The large amount of purified recombinant protein and protein complex produced allows for the use of these proteins in a robust screening assay to identify agents that are able to inhibit the interaction between Vif and Cul5. In one embodiment, the composition is a complex comprising ElonginB, ElonginC, Vif, and CBFβ.

It is discovered herein that co-expression of CBFβ with Vif allows production of: (i) large quantities of soluble Vif, (ii) a Vif protein that is far more functional in terms of binding to Cullin 5 than previously known in the art, and (iii) this functional form of full-length Vif can be expressed and purified at a modest expense from E. coli bacteria. Thus, co-expression of CBFβ allows for the stable production of full-length Vif and the production of a tetramer complex of ElonginB/ElonginC/Vif/CBFβ that comprises full-length Vif. Production of such a complex is useful in a screening assay along with recombinant Cul5 to identify compositions that prevent or inhibit Vif-Cul5 binding.

In one embodiment, ElonginB, ElonginC, Vif, CBFβ, and Cul5 are produced from individual expression vectors. In another embodiment, more than one of ElonginB, ElonginC, Vif, CBFβ, and Cul5 are expressed from the same expression vector. For example, in one embodiment, ElonginB and ElonginC are co-expressed from the same vector. For example, such a vector can comprise two multiple cloning sites (MCS) each of which is preceded by a promoter

In one embodiment, ElonginB comprises residues 1-118 of ElonginB (SEQ ID NO: 23). In one embodiment, ElonginC comprises residues 17-112 of ElonginC (SEQ ID NO: 24). In one embodiment Vif comprises full-length Vif comprising residues 1-192 of HXB2 subtype Vif (SEQ ID NO: 25). However, the invention is not limited to the expression of Vif from any particular HIV-1 isolate. Rather, the present invention encompasses the expression of full-length Vif from any isolate including, but not limited to HXB2 and HXB3. In another embodiment, Vif comprises a C-terminal domain of Vif (Vif_{C}), comprising residues 95-192 of Vif (SEQ ID NO: 26). In one embodiment, CBFβ, as used herein, comprises the amino acid sequence of SEQ ID NO: 27. In one embodiment, one or more of ElonginB, ElonginC, Vif, and CBFβ comprise a reporter or tag sequence that is used for purification purposes or for detection purposes in a screening assay. In certain embodiments, the mammalian or viral proteins of the present invention include variants of the sequences described herein, including for example variants with functional or non-functional mutations, splice variants, species variants, and the like.

In one embodiment, in order to evaluate the Vif-mediated interaction between Cul5 and ElonginB/ElonginC, a construct comprising the N-terminal domain of Cullin5 that encompasses the Vif interaction domain is used. In some embodiments, this construct is referred to as Cul5(N). In one embodiment Cul5(N) comprises residues 2-384 of Cul5. This is because, in some instances, expression of full-length Cul5 from is hindered by its large size and inherent insolubility of the globular C-terminal domain, which must be produced in the presence of rbx2 protein. However, in some embodiments, full-length Cul5 with rbx2, or fragments thereof, is used. In some embodiments, to improve solubility, two point mutations are introduced within the Cul5 fragment. The point mutations are V341R and L345D (Zheng et al, 2002, Nature, 416: 703-709). In one embodiment, the amino acid sequence of Cul5(N) used herein comprises SEQ ID NO: 28. In one embodiment, the amino acid sequence of Cul5(N) used herein comprises SEQ ID NO: 29. In other embodiments, full-length Cul5 is used. For purification purposes, a reporter or tag can be engineered to the Cul5 fragment.

In one embodiment, ElonginB/ElonginC is expressed from a pETDuet-1 expression vector. In one embodiment, full-length Vif is expressed from a pCDFDuet-1 expression vector. In one embodiment, CBFβ is expressed from a pRSFDuet-1 expression vector. In one embodiment, Cul5(N) is expressed from a pRSFDuet-1 expression vector. However, as would be understood by the skilled artisan, any suitable expression vector may be used to produce the desired peptides of the present invention.

The present disclosure is the first time large-scale production of a pure complex comprising, ElonginB, ElonginC, full-length Vif, and CBFβ has been successfully demonstrated, which is fully competent to avidly bind Cul5. Further, the data presented elsewhere herein quantifies the exceptionally high affinity of Cul5 for the EloB/EloC/Vif/CBFβ tetramer that comprises full-length Vif. The level of protein production is suitable for at least in vitro high-throughput screening. Based on the information provided herein, the polypeptides of the invention can be produced recombinantly using standard techniques well known to those of skill in the art or produced by a host cell. For example, the sequences of Vif, ElonginB, ElonginC, CBFβ, Cul5 are known and can be used to engineer the polypeptides of the invention. The nucleic acid sequence may be optimized to reflect particular codon “preferences” for various expression systems according to methods known in the art.

In general, fusion polypeptides of the invention can be produced by preparing a fused gene comprising a first DNA segment and a second DNA segment. Each fused gene is assembled in, or inserted into an expression vector. Recipient cells capable of expressing the gene products are then transfected with the genes. The transfected recipient cells are cultured under conditions that permit expression of the incorporated genes and the expressed fusion proteins are harvested.

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage et. al., 1981 Tetrahedron Letters. 22: 1859-1862.

Once a nucleic acid encoding a desired polypeptide is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook et al., Molecular Cloning: A Laboratory Manual, volumes 1-3 (3^{rd} ed., Cold Spring Harbor Press, NY 2001).

Once the nucleic acid for a desired polypeptide is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the polypeptides of the invention.

The present invention also provides for analogs of polypeptides of the invention. Analogs may differ from naturally occurring proteins or polypeptides by conservative amino acid sequence differences or by modifications that do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups: (a) glycine, alanine; (b) valine, isoleucine, leucine; (c) aspartic acid, glutamic acid; (d) asparagine, glutamine; (e) serine, threonine; (f) lysine, arginine; (g) phenylalanine, tyrosine.

Modifications (that do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes that affect glycosylation, e.g., mammalian glycosylating or deglycosylating enzymes. Also embraced are sequences that have phosphorylated amino acid residues, e.g., phosphotyrosine, phosphoserine, or phosphothreonine.

The present invention should also be construed to encompass “mutants,” “derivatives,” and “variants” of the peptides of the invention (or of the DNA encoding the same) in which such mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the polypeptides disclosed herein, in that the peptide has biological/biochemical properties.

### Vectors

Nucleic acids encoding the desired polypeptide or equivalents may be replicated in wide variety of cloning vectors in a wide variety of host cells.

In brief summary, the expression of natural or synthetic nucleic acids encoding a desired polypeptide will typically be achieved by operably linking a nucleic acid encoding the desired polypeptide or portions thereof to a promoter, and incorporating the construct into an expression vector. The vectors can be suitable for replication and integration. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In some aspects, the expression vector is selected from the group consisting of a viral vector, a bacterial vector, and a mammalian cell vector. Numerous expression vector systems exist that comprise at least a part or all of the compositions discussed above. Prokaryote- and/or eukaryote-vector based systems can be employed to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., Molecular Cloning: A Laboratory Manual, volumes 1-3 (3^{rd} ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (e.g., WO 01/96584; WO 01/29058; and U.S. Pat. No. 6,326,193).

For expression of the polypeptides of the invention or portions thereof, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (e.g., U.S. Pat. No. 4,683,202, U.S. Pat. No. 5,928,906).

Naturally, it will be important to employ a promoter and/or enhancer that effectively directs the expression of the DNA segment in the cell type, organelle, and organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of the polypeptides of the invention or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The invention includes a tag polypeptide that can be covalently linked thereto to the polypeptides of the invention. That is, the invention encompasses a recombinant nucleic acid wherein the nucleic acid encoding the tag polypeptide is covalently linked to the nucleic acid of the polypeptides of the invention. Such tag polypeptides are well known in the art and include, for instance, green fluorescent protein (GFP), myc, myc-pyruvate kinase (myc-PK), His₆, maltose binding protein (MBP), an influenza virus hemagglutinin tag polypeptide, a flag tag polypeptide (FLAG), a glutathione-S-transferase (GST) tag polypeptide, REACH2, sREACh, and a mEGFP protein. However, the invention should in no way be construed to be limited to the nucleic acids encoding the above-listed tag polypeptides. Rather, any nucleic acid sequence encoding a polypeptide that may function in a manner substantially similar to these tag polypeptides should be construed to be included in the present invention. Further, in some embodiments, addition of a tag polypeptide facilitates isolation and purification of the “tagged” protein such that the protein of the invention can be produced and purified readily.

### Methods of Introduction and Expression

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

In one embodiment, a vector or vectors encoding Vif, ElonginB, ElonginC, and CBFβ are transformed into E. coli cells. In one embodiment, a vector encoding Cul5(N) is transformed into E. coli cells.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, volumes 1-3 (3rd ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape.

Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Certain post-translational modifications are the result of the action of recombinant host cells on the expressed polypeptide. Glutaminyl and aspariginyl residues are frequently post-translationally deamidated to the corresponding glutamyl and aspartyl residues. Alternatively, these residues are deamidated under mildly acidic conditions. Either form of these residues falls within the scope of this invention.

Other post-translational modifications include hydroxylation of proline and lysine, phosphorylation of hydroxyl groups of seryl, threonyl or tyrosyl residues, methylation of the α-amino groups of lysine, arginine, and histidine side chains (T E Creighton (1983) Proteins: Structure and Molecular Properties, W.H. Freeman & Co., San Francisco, pp. 79-86).

Regardless of the method used to introduce exogenous nucleic acids into a host cell or otherwise expose a cell to the nucleic acid, in order to confirm the presence of the recombinant DNA sequence in the host cell, a variety of assays may be performed. Such assays include, for example, “molecular biological” assays well known to those of skill in the art, such as Southern and Northern blotting, reverse transcription polymerase chain reaction (RT-PCR) and PCR; “biochemical” assays, such as detecting the presence or absence of a particular peptide, e.g., by immunological means (ELISAs and Western blots).

In one embodiment, transformed or transfected cells are permitted to grow for a period of time suitable for protein expression. In one embodiment, cells are cultured in culture media that permits cell growth and/or protein expression. The precise type or formulation of culture media is dependent on the type of cell used to express the protein of the invention. For example, in one embodiment, E. coli cells transformed with a vector or vectors encoding the proteins of the invention are grown in Luria-Bertani (LB) media. In some embodiments, the cells are stimulated or induced to initiate protein expression. For example, in one embodiment, protein expression is induced by the administration of IPTG. In another embodiment, expression is induced through autoinduction in a lactose-containing media.

In certain embodiments, protein or protein complexes are isolated and purified from the expression system. Purification of the protein or protein complex may be carried out by any method known in the art. For example, in one embodiment, the expressed protein or protein complex may by purified through the use of compositions and methods that specifically detect a protein tag. In one embodiment, the purity of the purified protein or protein complex is greater than 90%. In another embodiment, the purity of the purified protein or protein complex is greater than 95%. In another embodiment, the purity of the purified protein or protein complex is greater than 98%.

### Screening Assay

In one aspect, the present invention is directed to a screening assay to identify compounds that prevent or inhibit Vif-Cul5 binding. As described elsewhere herein, it has been discovered that full-length Vif protein can be produced when co-expressed with CBFβ. In one embodiment, full-length Vif is produced in the context of an ElonginB/ElonginC/Vif/CBFβ tetramer complex to produce milligram quantities of active, purified, soluble, and functional protein. Moreover, the production of a soluble Cullin5 protein, sufficient to interact with Vif, provides an opportunity to initiate in vitro drug discovery assays. The assay is useful for identifying antiviral compounds that selectively bind to Vif or selectively inhibit Vif-mediated interactions between Cullin 5 and EloB/C, which is necessary for ubiquitination and degradation of the innate antiviral proteins APOBEC3G and related A3 family members. The assays described here are unique and are an enabling technology for the HIV/AIDS drug discovery industry.

The invention provides a method for identifying compounds that bind to Vif when the ElonginB/ElonginC/Vif/CBFβ tetramer complex is used in assays to contact chemistries from a library of compounds. In one embodiment, the ElonginB/ElonginC/Vif/CBFβ tetramer complex with an appropriate tag (such as GST, poly Histine, or epitope tag, etc.) is immobilized on a solid support and interacted with compounds with chemical libraries with the expressed intent of identify those compounds that bound to Vif.

The invention provides a method of identifying compounds that block the interaction between Cul5 and Vif. Without wishing to be bound by any particular theory, it is believed that blocking the interaction between Cul5 and Vif protects the infected cell's own innate immune factors, such as A3G. That is, under normal circumstances, HIV-1 infection leads to the production of the viral protein Vif. This viral factor is essential to evade the host's own A3G and A3-related defense factors. Vif works in concert with the cell's own ubiquitin ligase machinery to promote degradation of A3G and A3-related antiviral factors by the 26S proteasome. This requires a direct interaction between the HIV-1 protein Vif and Cullin 5.

Accordingly, the invention includes compounds identified by the screening methods that block the virus/host protein interaction. These compounds are considered antiviral compounds because they prevent Vif-APOBEC3G or Vif-APOBEC3F from interacting with Cullin 5 and the other components of the ubiquitination machinery. Consequently APOBEC3G and A3-related proteins are not destroyed by Vif and the increased intracellular abundance of these host-defense factors enables them to enter nascent viral particles from which point the host-defense factors are positioned to interact with viral replication complexes and assemble with viral particles following infection and thereby block viral infectivity.

One aspect of the invention is a method for identifying an agent (e.g. screening putative agents for one or more that elicits the desired activity) that inhibits the infectivity of a lentivirus (e.g., a lentivirus which expresses a Vif protein). Typical such lentiviruses include, e.g., SW, SHIV and/or HIV. The method takes advantage of the successful production of large-scale amounts of recombinant full-length Vif protein. Production of full-length Vif allow for assays for detecting an agent that is capable of interfering with the interaction between Vif and Cul5. The present assay allows for identification of an agent that acts anywhere along Vif protein. Such identification has otherwise been impossible, due to the ability to only recombinantly produce Vif fragments. An agent that interferes with Vif-Cul5 binding would be expected to inhibit the formation of a Vif/Cul5 complex thereby inhibiting the infectivity of a lentivirus that expresses a Vif protein. In some instances, because the assay is Vif-mediated or otherwise Vif dependent (Vif is not found in other cellular proteins), such an agent would not be expected to interfere with the function of cellular proteins and thus would be expected to elicit few, if any, side effects as a result of the binding.

In certain embodiments, the method comprises: (a) contacting a putative inhibitory agent with a mixture comprising Vif and Cul5 under conditions that are effective for Vif/Cul5 complex formation; and (b) detecting whether the presence of the agent decreases the level of Vif/Cul5 complex formation. In some instances, the agent binds to Vif and thereby inhibits Vif/Cul5 complex formation. In another instance, the agent binds to Cul5 and thereby inhibits Vif/Cul5 complex formation. Any of a variety of conventional procedures can be used to carry out such an assay. In certain embodiments, the Vif of the screening method is in the context of the Vif/EloB/EloC/CBFβ complex.

In certain embodiments, the method comprises contacting the putative inhibitory agent to a mixture comprising one of Vif or Cul5 and then contacting the mixture with the other of Vif or Cul5. For example, in certain instances, a screening method is enhanced by adding a potential inhibitor of protein-protein binding to a mixture comprising a first protein, followed by adding the second protein (Bauman et al., Top Curr Chem, 317: 181-200). In one embodiment, the method comprises (a) contacting a putative inhibitory agent with a mixture comprising Vif, (b) contacting Cul5 to the mixture, and (c) detecting whether the presence of the agent inhibits the level of Vif/Cul5 complex formation. In one embodiment the method comprises (a) contacting a putative inhibitory agent with a mixture comprising Cul5, (b) contacting Vif to the mixture, and (c) detecting whether the presence of the agent inhibits the level of Vif/Cul5 complex formation. Any quenched complex would be seen as unresponsive to the agent whereas a retention of fluorescence is indicative of the binding of the agent that precludes pentamer formation, thereby indicating that the agent is an inhibitor of Vif-Cul5 binding.

The invention encompasses methods to identify a compound that inhibits the interaction between Vif and Cul5. In one embodiment, the invention provides an assay for determining the binding between Cul5 with Vif, wherein Vif is in the context of ElonginB/ElonginC/Vif/CBFβ complex. The method includes contacting recombinant Vif and Cul5 in the presence of a candidate compound. Detecting inhibition or a reduced amount of Vif/Cul5 complex in the presence of the candidate compound compared to the amount of Vif/Cul5 complex in the absence of the candidate compound is an indication that the candidate compound is an inhibitor of Vif/Cul5 interaction.

Based on the disclosure presented herein, the screening method of the invention is applicable to a robust Förster quenched resonance energy transfer (FqRET) assay for high-throughput compound library screening in microtiter plates. The assay is based on selective placement of chromoproteins or chromophores that allow reporting on complex formation between the ElonginB/ElonginC/Vif/CBFβ protein complex and Cul5 in vitro. For example, an appropriately positioned FRET donor and FRET quencher will results in a “dark” signal when the pentameric complex is formed between ElonginB/ElonginC/Vif/CBFβ and Cullin5. In one embodiment, the FqRET assay of the invention includes one of ElonginB/ElonginC/Vif/CBFβ and Cullin5 comprising a FRET donor, while the other comprises a FRET quencher. Examples of FRET donors and FRET quenchers are well known in the art. In one embodiment, the ElonginB/ElonginC/Vif/CBFβ tetramer complex comprises mEGFP acting as a FRET donor. In one embodiment EGFP is expressed in the tetramer in the form of a fusion protein comprising at least one of ElonginB, ElonginC, Vif, or CBFβ fused to mEGFP. In one embodiment, the mEGFP tagged tetramer comprises at least one of ElonginB-mEGFP, ElonginC-mEGFP, Vif-mEGFP, or CBFβ-mEGFP. In one embodiment, Cul5 comprises sREACh, acting as a FRET acceptor and quencher. However, the present assay is not limited as the precise arrangement of the FRET donor and quencher. Rather, any arrangement that results in a “dark” signal when the pentameric ElonginB/ElonginC/Vif/CBFβ/Cul5 complex is formed and a detectable signal when formation of the pentameric complex is inhibited may be used in the present assay.

In one embodiment, the tetramer complex comprises ElonginB-mEGFP having an amino acid sequence of SEQ ID NO: 31. In one embodiment, the tetramer complex comprises CBFβ-mEGFP having an amino acid sequence of SEQ ID NO: 33. In one embodiment, the tetramer complex comprises Vif-mEGFP having an amino acid sequence of SEQ ID NO: 35.

In one embodiment, the tagged Cul5 comprises sREACh tagged to the N-terminus of Cul5(N), and has an amino acid sequence of SEQ ID NO: 37. In one embodiment, the tagged Cul5 comprises sREACh tagged to the C-terminus of Cul5(N), and has an amino acid sequence of SEQ ID NO: 39. In certain embodiments, the tagged Cul5 comprises a flexible linker peptide between the sREACh tag and Cul5.

In one embodiment, the screening method of the invention comprises a mixture comprising sREACh-Cul5(N) and tetramer complex comprising Vif-mEGFP, ElonginB, ElonginC, and CBFβ. It is demonstrated herein that sREACh-Cul5(N) effectively quenches the FRET signal from the labeled comprising Vif-mEGFP/ElonginB/ElonginC/CBFβ complex. Thus, the method of the invention comprises identifying a test compound which reduces the amount of quenching, as indicated by an increase in fluorescence compared to conditions in which the test compound is absent, thereby indicating that the compound inhibits the binding of Cul5 to Vif/ElonginB/ElonginC/CBFβ complex.

In one embodiment, the assay comprises expression of a recombinant complex comprising full-length HXB2 Vif (residues 1-192). As described elsewhere herein, production of full-length functionally interacting Vif is predicated upon the co-expression of CBFβ. The identification of methods to produce milligram quantities of the relevant protein complexes for screening, as well as quantitative validation of the binding between Cul5(N) and the ElonginB/ElonginC/full-length Vif/CBFβ complex is superior than prior methods that do not incorporate the essential protein CBFβ. Experiments presented elsewhere herein reveal that the inclusion of CBFβ in the context of full-length Vif/EloB/EloC (produces an apparent equilibrium K_{d} of 5±2 nM), which is 65-fold more avid in binding relative to the Cul5 interaction with truncated Vif in the tripartite complex comprising Vif(95-192)/EloB/EloC (K_{d} of 327±40 nM). Additional demonstrations of the quantification of this high-affinity complex in terms of heat capacity changes are described elsewhere herein.

The screening methods should not be limited solely to the assays disclosed herein. Rather, the recombinant proteins of the invention can be used in any assay, including other high-throughput screening assays that are applicable to the screening of agents that regulate the binding between to proteins. Thus, the invention encompasses the use of the recombinant proteins of the invention in any assay that is useful for detecting an agent that interferes with protein-protein interactions. Furthermore, the screening methods should also not be limited to identification of a Cullin5-Vif antagonist as a Vif-ElonginB antagonist, a Vif-ElonginC antagonist, and a Vif-CBFβ antagonist are also envisioned and these would also have value as to antiviral compounds that prevent ubiquitination and degradation of APOBEC3G and related family members.

The skilled artisan would also appreciate, in view of the disclosure provided herein, that standard binding assays known in the art, or those to be developed in the future, can be used to assess the binding of Vif with Cul5 using the recombinant proteins of the invention in the presence or absence of the test compound to identify a useful compound. Thus, the invention includes any compound identified using this method. Exemplary compounds that may be identified as inhibitors include, but are not limited to, nucleic acids, oligomers, peptides, antibodies, small molecules, aptamers, and the like.

The screening method includes contacting a mixture comprising recombinant Vif and Cul5 with a test compound and detecting the presence of the Vif/Cul5 complex, where a decrease in the level of Vif/Cul5 complex compared to the amount in the absence of the test compound or a control indicates that the test compound is able to inhibit the binding between Vif and Cul5. In certain embodiments, the control is the same assay performed with the test compound at a different concentration (e.g. a lower concentration), or in the absence of the test agent, etc. In certain embodiments, the mixture comprises an appropriate buffer which allows for the screening of Vif/Cul5 binding. In one embodiment, the mixture comprises a detergent, which in certain instances is required for the stability of the assay. For example, in one embodiment, the mixture comprises at least one of Brij 35, Tween 20, Triton X-100, and glycerol. In one embodiment, the solution comprises any agent that reduces protein aggregation, including, but not limited to bovine serum albumin (BSA).

Without wishing to be bound by any particular theory, it is believed that the Vif/Cul5 complex contains a ceiling level of complex formation because the presence the two proteins have a propensity to bind with one another and in the absence of the Cullin5 scaffold, E2 ligase cannot ubiquinate Vif or APOBEC3G (and related A3 proteins) and thus Vif will not mediate the destruction of these antiretroviral proteins. The activity of a test compound can be measured by determining whether the test compound can decrease the level of Vif/Cul5 complex formation.

Determining the ability of the test compound to interfere with the formation of the Vif/Cul5 complex can be accomplished, for example, by coupling the Vif protein or the Cul5 protein with a tag, radioisotope, or enzymatic label such that the Vif/Cul5 complex can be measured by detecting the labeled component in the complex. For example, a component of the complex (e.g., Vif or Cul5) can be labeled with ³²P, ¹²⁵I, ³⁵S, ¹⁴C or ³H, either directly or indirectly, and the radioisotope detected by direct counting of radioemission or by scintillation counting. Alternatively, a component of the complex can be enzymatically labeled with, for example, horseradish peroxidase, alkaline phosphatase, or luciferase, and the enzymatic label is then detected by determination of conversion of an appropriate substrate to product.

Determining the ability of the test compound to interfere with the Vif/Cul5 complex can also be accomplished using technology such as real-time Biomolecular Interaction Analysis (BIA) as described in Sjolander et al., 1991, Anal. Chem. 63:2338-2345 and Szabo et al., 1995, Curr. Opin. Struct. Biol. 5:699-705. BIA is a technology for studying biospecific interactions in real time, without labeling any of the interactants (e.g., BIAcore, BIAcore International AB, Uppsala, Sweden). Changes in the optical phenomenon of surface plasmon resonance (SPR) can be used as an indication of real-time reactions between biological molecules.

In more than one embodiment of the methods of the present invention, it may be desirable to immobilize either Vif or Cul5 to facilitate separation of complexed from uncomplexed forms of one or both of the molecules, as well as to accommodate automation of the assay. The effect of a test compound on the Vif/Cul5 complex can be accomplished using any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/target fusion proteins can be adsorbed onto glutathione sepharose beads (Sigma Chemical, St. Louis, Mo.) or glutathione-derivatized micrometer plates, which are then combined with the other corresponding component of the Vif/Cul5 complex in the presence of the test compound. The mixture is incubated under conditions conducive to complex formation (e.g., at physiological conditions for salt and pH). Following incubation, the beads or microtiter plate wells are washed to remove any unbound material, the matrix is immobilized in the case of beads, and the formation of the complex is determined either directly or indirectly, for example, as described elsewhere herein.

Other techniques for immobilizing proteins on matrices can also be used in the screening assays of the invention. For example, either Vif or Cul5 can be separated from a mixture using conjugated biotin and streptavidin. For example, biotinylated Cul5 or Vif can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates. Alternatively, antibodies reactive with for example Cul5 or Vif, but do not interfere with binding of Vif with Cul5 can be derivatized to the wells of the plate. Methods for detecting such complexes, in addition to those described above for the GST-immobilized complexes, include immunodetection of complexes using reactive antibodies, as well as enzyme-linked assays.

The assay of the present invention also includes a cell-based assay, wherein the recombinant ElonginB/ElonginC/Vif/CBFβ tetramer complex and recombinant Cul5 protein are introduced into a cell. Any suitable cell may be used for the cell-based assay including, but not limited to, prokaryotic cells, eukaryotic cells, and mammalian cells. In one embodiment, the cell-based assay comprises one or more cells derived from a cell line including, but not limited to, HEK 293T, CHO, BHK, VERO, HeLa, COS, MDCK, NS0 and W138. In one embodiment, the cell based assay comprises one or more primary cells isolated from a subject (e.g. a mammal). For example, in one embodiment, the assay comprises the use of a CD4+ cell isolated from a subject. In one embodiment, the cell based screen comprises an in vivo screening assay, wherein the recombinant protein is introduced into one or more cells in animal. In some embodiments, a cell based assay is used as a secondary screen on test compounds identified as inhibitors of Vif-Cul5 binding in an in vitro screening assay.

The test compounds can be obtained using any of the numerous approaches in combinatorial-library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical that new chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Therapeutic Compositions

In one embodiment, the present invention is directed to a composition that inhibits or prevents the binding between Vif and Cul5. In one embodiment, the composition is a peptide, antibody, antibody fragment, nucleic acid, peptidomimetic, small molecule, aptamer, or any combination thereof. As described elsewhere herein, the present invention is partly based upon the identification of specific sequences within Vif and Cul5 that mediate binding between Vif and Cul5. In one embodiment, the composition comprises a peptide inhibitor that competes with Vif and/or Cul5, thereby preventing Vif-Cul5 binding. In another embodiment, the composition directly or indirectly binds to an epitope of Vif and/or Cul5 thereby preventing Vif-Cul5 binding. In one embodiment, the epitope is a region of Vif as defined by at least one of SEQ ID NOs: 1-8. In another embodiment, the epitope is a region of Cul5 as defined by at least one of SEQ ID NOs: 9-11. In one embodiment, the composition comprises an agent identified in the screening assay of the invention as an agent that prevents or inhibits Vif-Cul5 binding. In one embodiment, the composition inhibits Vif-CBFβ binding, thereby reducing the affinity of Vif-Cul5 binding.

### Inhibitors

In one embodiment, the present invention is related to a composition that prevents or inhibits Vif-Cul5 binding. The present invention is partly based upon hydrogen-deuterium exchange mass spectrometry (HDX-MS) performed on (i) the ElonginB/ElonginC/Vif/CBFβ tetramer complex, (ii) Cullin 5, and (iii) the ElonginB/ElonginC/Vif/CBFβ/Cullin 5 pentameric complex, which provided the identification of specific sequences within Vif and Cul5 that were critical in the formation of the ElonginB/ElonginC/Vif/CBFβ/Cullin 5 pentameric complex.

The regions of Vif that are critical for Vif-Cul5 binding are: (a) ¹⁹RTWKSLVKHHMYVSGKARGWF³⁹ (SEQ ID NO: 1), (b) ⁸⁹WRKKRYSTQVDPEL¹⁰⁴ (SEQ ID NO: 2), (c) ¹¹³FD¹¹⁴ (SEQ ID NO: 3), (d) ¹¹⁵CF¹¹⁶ (SEQ ID NO: 4), (e) ¹¹⁷DSAIRKALL¹²⁵ (SEQ ID NO: 5), (f) ¹²⁸IVSPRCEY¹³⁵ (SEQ ID NO: 6), (g) ¹³⁶QAGHNKVGSLQ¹⁴⁶ (SEQ ID NO: 7), and (h) ¹⁵³LITPKKIKPPLPSPTKL¹⁶⁹ (SEQ ID NO: 8).

The regions of Cul5 that are critical for Vif-Cul5 binding are (A) ³⁰LRQESVTKQQW⁴⁰ (SEQ ID NO: 9), (B) ⁴³LFSDVHAVCL⁵² (SEQ ID NO: 10), and (C) ⁵⁵DKGPAKIHQAL⁶⁵ (SEQ ID NO: 11).

In one embodiment, the composition of the present invention comprises a peptide, or derivative thereof that inhibits or prevents Vif-Cul5 binding. In one embodiment, the composition comprises at least one of SEQ ID NOs: 1-11. In one embodiment the composition competes with Vif and/or Cul5, thereby inhibiting or preventing Vif-Cul5 binding.

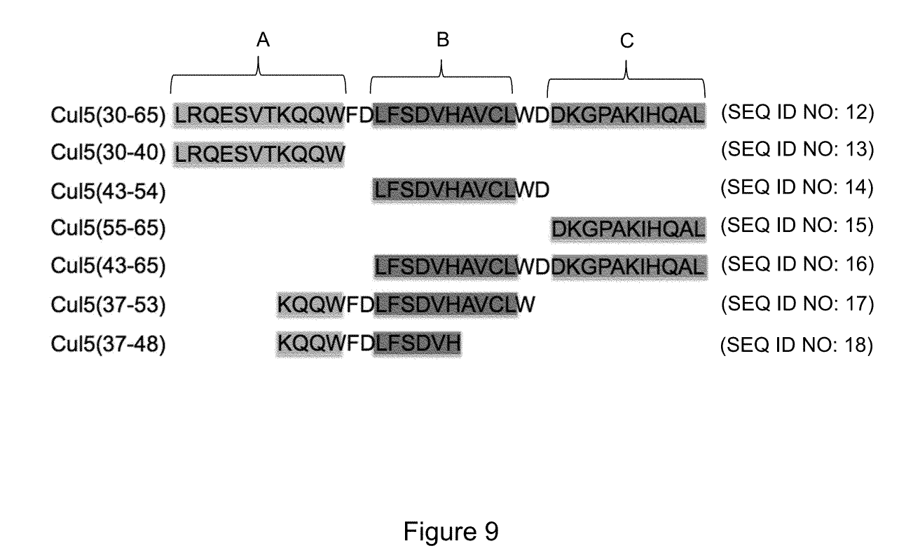

In one embodiment, the composition of the present invention comprises a peptide comprises an amino acid sequence of Cul5(30-65) (SEQ ID NO: 12), or a fragment thereof. For example, in certain embodiments, the peptide comprises an amino acid sequence comprising at least one of Cul5(30-65)(SEQ ID NO: 12), Cul5(30-40)(SEQ ID NO: 13), Cul5(43-54)(SEQ ID NO: 14), Cul5(55-65)(SEQ ID NO: 15), Cul5(43-65)(SEQ ID NO: 16), Cul5(37-53)(SEQ ID NO: 17), and Cul5(37-48)(SEQ ID NO: 18). SEQ ID NOs: 12-18 are depicted in FIG. 9. It is demonstrated herein that peptides having an amino acid sequence of one of SEQ ID NOs: 12-18 bind to the ElonginB/ElonginC/Vif/CBFβ tetramer complex, and therefore serve as inhibitors of Cul5 binding to the tetramer complex.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The polypeptides of the invention can be post-translationally modified. For example, post-translational modifications that fall within the scope of the present invention include signal peptide cleavage, glycosylation, acetylation, isoprenylation, proteolysis, myristoylation, phosphorylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs that have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The term “functionally equivalent” as used herein refers to a polypeptide according to the invention that preferably retains at least one biological function or activity of the specific amino acid sequence of a peptide of the invention.

A peptide inhibitor of the invention may be conjugated with other molecules, such as proteins, to prepare fusion proteins. This may be accomplished, for example, by the synthesis of N-terminal or C-terminal fusion proteins provided that the resulting fusion protein retains the functionality of protein inhibitor of the invention.

(a) Tags

In a particular embodiment of the invention, the polypeptide of the invention further comprises the amino acid sequence of a tag. The tag includes but is not limited to: polyhistidine tags (His-tags) (for example H₆ and H₁₀, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

(b) Leader and Secretory Sequences

Accordingly, the polypeptides of the invention can be fused to another polypeptide or tag, such as a leader or secretory sequence or a sequence which is employed for purification or for detection. In a particular embodiment, the polypeptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the polypeptide sequence, thus permitting the removal of the binding tag after incubation with this specific enzyme and thus facilitating the purification of the corresponding protein of interest.

(c) Targeting Sequences

The invention also relates to a peptide of the invention fused to, or integrated into, a target protein, and/or a targeting domain capable of directing the peptide to a desired cellular component or cell type or tissue. The peptide may also contain additional amino acid sequences or domains. The peptides are recombinant in the sense that the various components are from different sources, and as such are not found together in nature (i.e. are heterologous).

The targeting domain can be a membrane spanning domain, a membrane binding domain, or a sequence directing the peptide to associate with for example vesicles or with the nucleus. The targeting domain can target the peptide of the invention to a particular cell type or tissue. For example, the targeting domain can be a cell surface ligand or an antibody against cell surface antigens of a target tissue (e.g. neuron or tumor antigens). A targeting domain may target a peptide of the invention to a cellular component.

(d) Intracellular Targeting

Combined with certain formulations, such peptides can be effective intracellular agents. However, in order to increase the efficacy of such peptides, the peptide of the invention can be provided a fusion peptide along with a second peptide which promotes “transcytosis”, e.g., uptake of the peptide by cells. To illustrate, the peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the peptide of the invention can be provided a fusion polypeptide with all or a portion of the antenopedia III protein.

To further illustrate, the peptide of the invention (or peptidomimetic) can be provided as a chimeric peptide which includes a heterologous peptide sequence (“internalizing peptide”) which drives the translocation of an extracellular form of a peptide sequence across a cell membrane in order to facilitate intracellular localization of the peptide. In this regard, the peptide sequence is one which is active intracellularly. The internalizing peptide, by itself, is capable of crossing a cellular membrane by, e.g., transcytosis, at a relatively high rate. The internalizing peptide is conjugated, e.g., as a fusion protein, to the peptide of the invention. The resulting chimeric peptide is transported into cells at a higher rate relative to the peptide alone to thereby provide a means for enhancing its introduction into cells to which it is applied.

In one embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193.

The present invention contemplates a peptide or peptidomimetic sequence as described herein, and at least a portion of the Antennapedia protein (or homolog thereof) sufficient to increase the transmembrane transport of the chimeric peptide, relative to the peptide or peptidomimetic, by a statistically significant amount.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell, 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8).

Another exemplary transcellular polypeptide can be generated to include a sufficient portion of mastoparan (T. Higashijima et al., (1990) J. Biol. Chem. 265:14176) to increase the transmembrane transport of the chimeric peptide.

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefore serve as an internalizing peptide for the subject transcellular peptides and peptidomimetics

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of peptides and peptidomimetics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa values within the range of pH 5-7, so that a sufficient uncharged membrane-binding domain will be present within the peptide at pH 5 to allow insertion into the target cell membrane.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore-forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

Mere membrane intercalation of an internalizing peptide may be sufficient for translocation of the peptide or peptidomimetic, across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

To further illustrate use of an accessory peptide, a phosphorylatable accessory peptide is first covalently attached to the C-terminus of an internalizing peptide and then incorporated into a fusion protein with the peptide or peptidomimetic of the invention. The internalizing peptide component of the fusion protein intercalates into the target cell plasma membrane and, as a result, the accessory peptide is translocated across the membrane and protrudes into the cytoplasm of the target cell. On the cytoplasmic side of the plasma membrane, the accessory peptide is phosphorylated by cellular kinases at neutral pH. Once phosphorylated, the accessory peptide acts to irreversibly anchor the fusion protein into the membrane. Localization to the cell surface membrane can enhance the translocation of the polypeptide into the cell cytoplasm.

Suitable accessory peptides include peptides that are kinase substrates, peptides that possess a single positive charge, and peptides that contain sequences which are glycosylated by membrane-bound glycotransferases. Accessory peptides that are glycosylated by membrane-bound glycotransferases may include the sequence x-NLT-x, where “x” may be another peptide, an amino acid, coupling agent or hydrophobic molecule, for example. When this hydrophobic tripeptide is incubated with microsomal vesicles, it crosses vesicular membranes, is glycosylated on the luminal side, and is entrapped within the vesicles due to its hydrophilicity (C. Hirschberg et al., (1987) Ann. Rev. Biochem. 56:63-87). Accessory peptides that contain the sequence x-NLT-x thus will enhance target cell retention of corresponding polypeptide.

In another embodiment of this aspect of the invention, an accessory peptide can be used to enhance interaction of the peptide or peptidomimetic of the invention with the target cell. Exemplary accessory peptides in this regard include peptides derived from cell adhesion proteins containing the sequence “RGD”, or peptides derived from laminin containing the sequence CDPGYIGSRC (SEQ ID NO. 19). Extracellular matrix glycoproteins, such as fibronectin and laminin, bind to cell surfaces through receptor-mediated processes. A tripeptide sequence, RGD, has been identified as necessary for binding to cell surface receptors. This sequence is present in fibronectin, vitronectin, C3bi of complement, von-Willebrand factor, EGF receptor, transforming growth factor beta, collagen type I, lambda receptor of E. Coli, fibrinogen and Sindbis coat protein (E. Ruoslahti, Ann. Rev. Biochem. 57:375413, 1988). Cell surface receptors that recognize RGD sequences have been grouped into a superfamily of related proteins designated “integrins”. Binding of “RGD peptides” to cell surface integrins will promote cell-surface retention, and ultimately translocation, of the polypeptide.

As described elsewhere herein, the internalizing and accessory peptides can each, independently, be added to the peptide or peptidomimetic of the invention by either chemical cross-linking or in the form of a fusion protein. In the instance of fusion proteins, unstructured polypeptide linkers can be included between each of the peptide moieties.

In general, the internalization peptide will be sufficient to also direct export of the polypeptide. However, where an accessory peptide is provided, such as an RGD sequence, it may be necessary to include a secretion signal sequence to direct export of the fusion protein from its host cell. In preferred embodiments, the secretion signal sequence is located at the extreme N-terminus, and is (optionally) flanked by a proteolytic site between the secretion signal and the rest of the fusion protein.

In certain instances, it may also be desirable to include a nuclear localization signal as part of the peptide.

In the generation of fusion polypeptides including the subject peptides, it may be necessary to include unstructured linkers in order to ensure proper folding of the various peptide domains. Many synthetic and natural linkers are known in the art and can be adapted for use in the present invention, including the (Gly₃Ser)₄ linker.

(e) Peptidomimetics

In other embodiments, the subject compositions are peptidomimetics of the peptides of the invention. Peptidomimetics are compounds based on, or derived from, peptides and proteins. The peptidomimetics of the present invention typically can be obtained by structural modification of the peptide sequence of the invention using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject peptides can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p 134). Also, see generally, Session III: Analytic and synthetic methods, in Peptides: Chemistry and Biology, G. R. Marshall ed., ESCOM Publisher: Leiden, Netherlands, 1988)

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the peptide. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to guide structure prediction of potential mimetopes by, for example, computer modeling, the predicted mimetope structures can then be produced by, for example, chemical synthesis, recombinant DNA technology, or by isolating a mimetope from a natural source (e.g., plants, animals, bacteria and fungi).

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a RLP or chimeric protein may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide of the invention conjugated with other molecules may be prepared by fusing, through recombinant techniques, the N-terminal or C-terminal of the peptide, and the sequence of a selected protein or selectable marker with a desired biological function. The resultant fusion proteins contain the peptide of the invention fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, the present invention provides a composition that directly or indirectly binds to an epitope of Vif or Cul5, thereby preventing or inhibiting Vif-Cul5 binding. In one embodiment, the epitope is a region of Vif or Cul5 that is known to be integral in mediating Vif-Cul5 binding. For example, in one embodiment, the epitope is a region of Vif defined by at least one of SEQ ID NOs: 1-8. In one embodiment, the epitope is a region of Cul5 defined by at least one of SEQ ID NOs: 9-11. In one embodiment, a composition that binds to a defined epitope of Vif or Cul5 is a peptide. In one embodiment, the composition that binds to an epitope of Vif or Cul5 is a peptide, antibody, antibody fragment, nucleic acid, peptidomimetic, small molecule, aptamer, or any combination thereof.

### Antibodies

In a particular embodiment, the composition of the invention is an antibody that specifically binds to an epitope of Vif or Cul5, sometimes referred herein as an antibody of the invention. In some embodiments, the antibody specifically binds to an epitope of Vif defined by at least one of SEQ ID NOs: 1-8. In other embodiments, the antibody specifically binds to an epitope of Cul5 defined by at least one of SEQ ID NOs: 9-11. Such antibodies include polyclonal antibodies, monoclonal antibodies, Fab and single chain Fv (scFv) fragments thereof, bispecific antibodies, heteroconjugates, human and humanized antibodies. Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity and are cleared rapidly from the blood.

The antibodies of the present invention may be polyclonal antibodies. Such polyclonal antibodies can be produced in a mammal, for example, following one or more injections of an immunizing agent, and preferably, an adjuvant. Typically, the immunizing agent and/or adjuvant will be injected into the mammal by a series of subcutaneous or intraperitoneal injections. The immunizing agent may include Vif or Cul5 or a fragment thereof or a fusion protein thereof. For example, in one embodiment, the immunizing agent includes a peptide comprising at least one of SEQ ID NOs: 1-11. Alternatively, a crude protein preparation that has been enriched for Vif or Cul5 or a fragment thereof can be used to generate antibodies. Such proteins, fragments or preparations are introduced into the non-human mammal in the presence of an appropriate adjuvant. If the serum contains polyclonal antibodies to undesired epitopes, the polyclonal antibodies are purified by immunoaffinity chromatography.

Alternatively, the antibodies may be monoclonal antibodies. Monoclonal antibodies may be produced by hybridomas, wherein a mouse, hamster, or other appropriate host animal, is immunized with an immunizing agent to elicit lymphocytes that produce or are capable of producing antibodies that will specifically bind to the immunizing agent, e.g. Kohler and Milstein, Nature 256:495 (1975) The immunizing agent will typically include the Vif or Cul5 protein or a fragment thereof or a fusion protein thereof and optionally a carrier. Alternatively, lymphocytes may be immunized in vitro. Generally, spleen cells or lymph node cells are used if non-human mammalian sources are desired, or peripheral blood lymphocytes (“PBLs”) are used if cells of human origin are desired. The lymphocytes are fused with an immortalized cell line using a suitable fusing agent, such as polyethylene glycol, to produce a hybridoma cell. In general, immortalized cell lines are transformed mammalian cells, for example, myeloma cells of rat, mouse, bovine or human origin. The hybridoma cells are cultured in a suitable culture medium that preferably contains one or more substances that inhibit the growth or survival of unfused, immortalized cells. The culture medium (supernatant) in which the hybridoma cells are cultured can be assayed for the presence of monoclonal antibodies directed against Vif or Cul5 by conventional techniques, such as by immunoprecipitation or by an in vitro binding assay, such as RIA or ELISA.

The monoclonal antibodies may also be made by recombinant DNA methods, such as those described in U.S. Pat. No. 4,816,567. DNA encoding the monoclonal antibodies of the invention can be isolated from the phosphorylated Elk-1-specific hybridoma cells and sequenced, e.g., by using oligonucleotide probes that are capable of binding specifically to genes encoding the heavy and light chains of murine antibodies. Once isolated, the DNA may be inserted into an expression vector, which is then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for the murine heavy and light chain constant domains for the homologous human sequences, or by covalently joining to the immunoglobulin coding sequence all or part of the coding sequence for a non-immunoglobulin polypeptide. The non-immunoglobulin polypeptide can be substituted for the constant domains of an antibody of the invention, or can be substituted for the variable domains of one antigen-combining site of an antibody of the invention to create a chimeric bivalent antibody.

The antibodies may also be monovalent antibodies. Methods for preparing monovalent antibodies are well known in the art. For example, in vitro methods are suitable for preparing monovalent antibodies. Proteolytic digestion of antibodies to produce fragments thereof, particularly, Fab fragments, can be accomplished using routine techniques known in the art.

Antibodies and antibody fragments characteristic of hybridomas of the invention can also be produced by recombinant means by extracting messenger RNA, constructing a cDNA library, and selecting clones which encode segments of the antibody molecule.

The antibodies of the invention may further comprise humanized antibodies or human antibodies. The term “humanized antibody” refers to humanized forms of non-human (e.g., murine) antibodies that are chimeric antibodies, immunoglobulin chains or fragments thereof (such as Fv, Fab, Fab′, F(ab′), or other antigen-binding partial sequences of antibodies) which contain some portions of the sequence derived from non-human antibodies. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Methods for humanizing non-human antibodies are well known in the art. Generally, a humanized antibody has one or more amino acids introduced into it from a source which is non-human in order to more closely resemble a human antibody, while still retaining the original binding activity of the antibody.

Heteroconjugate antibodies that comprise two covalently joined antibodies, are also within the scope of the present invention. Heteroconjugate antibodies may be prepared in vitro using known methods in synthetic protein chemistry, including those involving crosslinking agents. For example, immunotoxins may be prepared using a disulfide exchange reaction or by forming a thioether bond.

The antibodies of the invention are preferably specific for the Vif or Cul5 protein and so, do not bind peptides derived from other proteins with high affinity. The antibodies may be used as functional modulators, most commonly as antagonists. Preferably, antibody modulators of the invention are derived from monoclonal antibodies specific for Vif or Cul5 protein. Monoclonal antibodies capable of blocking or neutralizing Vif or Cul5 protein are generally selected by their ability to inhibit Vif-Cul5 binding.

Preferably, monoclonal antibodies, Fv fragments, Fab fragments, or other binding compositions derived from monoclonal antibodies of the invention have a high affinity to Vif or Cul5 protein. The affinity of monoclonal antibodies and related molecules to Vif or Cul5 protein may be measured by conventional techniques.

In one embodiment, the antibodies of the present invention include those cloned from a phage antibody library. For example, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab or scFv fragments on their surface. Phage that display the antibody of interest are selected by antigen binding and are propagated in bacteria to produce soluble human Fab or scFv immunoglobulin. Thus, in contrast to conventional monoclonal antibody synthesis, this procedure immortalizes DNA encoding human immunoglobulin rather than cells which express human immunoglobulin.

In one embodiment, the antibodies of the invention comprise anti-Vif or anti-Cul5 antibodies isolated using phage display. In another embodiment, the antibodies of the invention comprise anti-Vif or anti-Cul5 scFv mAbs isolated using phage display.

Phage display libraries allow for the in vitro identification of human antibody products directed against molecular targets. In one embodiment, the methods of the present invention provide that phage display libraries enable the in vitro identification of human antibody products directed against isolated Vif or Cul5 or fragment thereof.

Antibody phage display of the present invention provides the linkage between genotype and phenotype. In another embodiment, selection of phage clones of the present invention is based on binding affinity. In another embodiment, selection of phage clones of the present invention is based on binding specificity. In another embodiment, selection of phage clones of the present invention is based on functional activity of the displayed antibody (phenotype). In another embodiment, selection of phage clones of the present invention is based on binding affinity, specificity, and functional activity of the displayed antibody (phenotype). Each phage carries the DNA for the antibody it displays on its surface, the phenotype is directly linked to the antibody genotype (cDNA sequence). This enables rapid selection of clones displaying antibody chains with desirable characteristics with desirable affinity to a target antigen.

In another embodiment, the present invention provides a high affinity human antibody that is specific for Vif or Cul5. In another embodiment, the present invention provides that light chain shuffling while retaining the heavy chain variable region using phage display technology is able to modify the ultrafine specificity of the antigen-binding construct. In another embodiment, the present invention provides that heavy chain shuffling while retaining the light chain variable region using phage display technology is able to modify the ultrafine specificity of the antigen-binding construct. In another embodiment, the present invention provides that heavy chain promiscuity is limited in in vivo developed antibodies. In another embodiment, the present invention provides that light chain promiscuity is limited in in vivo developed antibodies. In another embodiment, the present invention allows such to form antigen-binding structures following random assortment of heavy and light chain variable regions. In another embodiment, the present invention provides that phage display technology selects against clones which are restricted with respect to light chain usage. In another embodiment, the present invention provides that phage display technology selects against clones which are restricted with respect to heavy chain usage. In another embodiment, the present invention provides that phage display technology represents a population which is restricted in this respect.

In one aspect, the antibodies of the present invention are useful for preventing or inhibiting Vif-Cul5 binding.

The present invention also includes small molecules that specifically bind to an epitope of Vif or Cul5 thereby preventing Vif-Cul5 binding. In one embodiment, the small molecule is identified in a screening assay, for example in the screening assay described elsewhere herein. In one embodiment, the small molecule is identified by evaluating its functional activity of preventing or reducing Vif-Cul5 binding. In another embodiment, the small molecule is identified by evaluating its functional activity of reducing viral infectivity. As would be understood by those skilled in the art, the present invention encompasses any composition that directly or indirectly prevents or inhibits Vif-Cul5 binding. As described elsewhere herein, in one embodiment, the composition competes with either Vif or Cul5 thereby preventing or inhibiting the formation of a Vif-Cul5 complex. In another embodiment, the composition binds to an epitope of Vif or Cul5, wherein the epitope is critical for Vif-Cul5 binding, thereby preventing or inhibiting Vif-Cul5 binding.

In one embodiment, the present invention provides a pharmaceutical composition comprising a composition of the invention, or a pharmaceutically acceptable salt, derivative or prodrug thereof together with a pharmaceutically acceptable carrier, adjuvant, or vehicle, for administration to a patient. The use of a composition of the invention in the manufacture of a pharmaceutical composition to prevent or treat viral infection constitutes another aspect of the invention.

In one embodiment, the present invention includes an isolated nucleic acid comprising a nucleotide sequence encoding an inhibitor (e.g. peptide inhibitor, antibody, antibody fragment, etc.) of the present invention. As discussed elsewhere herein, a nucleotide sequence encoding a protein of interest can comprise sequence variations with respect to the original nucleotide sequence. Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage et. al., 1981 Tetrahedron Letters. 22: 1859-1862. As discussed elsewhere herein, once a nucleic acid encoding a desired polypeptide is synthesized it may be amplified or cloned using standard procedures and the recombinant gene may be expressed in a variety of cell types. The present invention also includes a vector encoding the inhibitor (e.g. peptide inhibitor, antibody, antibody fragment, etc.) of the present invention. Vectors useful for expressing a peptide of interest is discussed elsewhere herein.

### Methods of Treatment

In one embodiment, the present invention provides methods of treating a disease, disorder, or condition associated with a viral infection. Preferably, the viral infection is associated with Vif. In one embodiment, the viral infection is HIV. The method comprises administering to a subject, such as a mammal, a therapeutically effective amount of a pharmaceutical composition that inhibits the interaction between Vif and Cullin5. In one embodiment, the subject is a human. However, the present invention is not limited to the treatment of a human subject. That is, the present invention encompasses the treatment of a viral infection in any suitable subject.

The invention includes compositions discussed elsewhere herein and compositions identified using the screening methods discussed elsewhere herein. Such a composition can be used as a therapeutic to treat an HIV infection or otherwise a disorder associated with Vif.

The ability for a composition to inhibit the interaction between Vif and Cullin5 can provide a therapeutic to protect or otherwise prevent viral infection, for example HIV infection.

Thus, the invention includes pharmaceutical compositions. Pharmaceutically acceptable carriers that are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, peritoneal, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, peritoneal, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

As used herein, “peritoneal administration” of a pharmaceutical composition includes any route of administration characterized by physical breaching of a tissue of a subject and administration of the pharmaceutical composition through the breach in the tissue. Peritoneal administration thus includes, but is not limited to, administration of a pharmaceutical composition by injection of the composition, by application of the composition through a surgical incision, by application of the composition through a tissue-penetrating non-surgical wound, and the like. In particular, peritoneal administration is contemplated to include, but is not limited to, subcutaneous, intraperitoneal, intramuscular, intrasternal injection, and kidney dialytic infusion techniques.

A pharmaceutical composition can consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Controlled- or sustained-release formulations of a pharmaceutical composition of the invention may be made using conventional technology.

Formulations of a pharmaceutical composition suitable for peritoneal administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for peritoneal administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for peritoneal administration, the active ingredient is provided in dry (i.e., powder or granular) form for reconstitution with a suitable vehicle (e.g., sterile pyrogen-free water) prior to peritoneal administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered as a bolus injection that provides lasting effects for at least one day following injection. The bolus injection can be provided intraperitoneally.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Core-Binding Factor β (CBFβ) Increases the Affinity Between Human Cullin 5 and HIV-1 Vif within an E3 Ligase Complex

HIV-1 Vif masquerades as a receptor for a cellular E3 ligase harboring ElonginB, ElonginC, and Cullin5 (EloB/C/Cul5) proteins that facilitate degradation of the antiretroviral factor A3G. This Vif-mediated activity requires human CBFβ in contrast to cellular substrate receptors. As presented herein, it was calorimetrically observed that Cul5 binds tighter to full-length Vif(1-192)/EloB/C/CBFβ (K_{d}=5±2 nM) than Vif(95-192)/EloB/C (K_{d}=327±40 nM), which cannot bind CBFβ. A comparison of heat-capacity changes supports a model wherein CBFβ prestabilizes Vif(1-192) relative to Vif(95-192), consistent with a stronger Cul5 interaction with Vif's C-terminal Zn²⁺-binding motif. The data presented herein suggests that an additional interface between Cul5 and an N-terminal region of Vif may exist, which has therapeutic-design implications.

The materials and methods used in the experiments are now described.

### Cloning of Vif_{C}/EloB/C and SOCS2_{SOCS-box}/EloB/C Expression Constructs

Bacterial expression of the ternary Vif_{C}/EloB/C complex entailed co-expression of an EloB₁₋₁₁₈-Vif₉₅₋₁₉₂ fusion protein and the EloC₁₇₋₁₁₂ protein from their DNA sequences, which were sub-cloned into MCS1 and MCS2 of pETDuet-1 (EMD4Biosciences), respectively. A 35 amino acid linker (ENLYFQSASGGHHHHGHH-HHTSGGENLYFQSGGGS) (SEQ ID NO: 19) was employed to fuse the C-terminus of EloB with residue 95 of Vif to impart solubility and to overcome stoichiometry problems that arose from use of independent open reading frames. This linker comprises two TEV protease cleavage sequences (ENLYFQS) (SEQ ID NO: 20) that flank a tandem HIS-tag (HHHHGHHHH) (SEQ ID NO: 21) that is punctuated by a Gly for flexibility. The E. coli expression-optimized gene encoding human EloB₁₋₉₈ was synthesized (BioBasic, Inc.) and cloned into MCS1 of a previously modified pETDuet-1 vector with 5′ NcoI and 3′ SacI restriction sites. The HXB-2 Vif₉₅₋₁₉₂ gene was cloned downstream of EloB with 5′ BamHI and 3′ XhoI restriction sites. Subsequently, a synthetic DNA sequence coding for EloB₉₉₋₁₁₈ and the 35 amino acid linker (IDT, Inc.) was cloned between EloB₁₋₉₈ and Vif₉₅₋₁₉₂ with 5′ SacI and 3′ BamHI restriction sites, thus generating the fusion gene for EloB₁₋₁₁₈-Vif₉₅₋₁₉₂. The human EloC₁₇₋₁₁₂ gene (Open Biosystems) was cloned subsequently into MCS2 using restriction enzymes NdeI and BglII, completing the expression construct pV_{C}EloBC. Expression of the ternary SOCS2_{SOCS-box}/EloB/C was analogous to that of Vif_{C}/EloB/C. The SOCS2_{SOCS-box}/EloB/C expression construct (pSOCS2EloBC) was generated by digesting pV_{C}EloBC with BamHI and XhoI to remove Vif₉₅₋₁₉₂, followed by ligation of the human SOCS2₁₅₈₋₁₉₈ synthetic gene (BioBasic, Inc.). All constructs were verified by DNA sequencing.

### Expression and Purification of Vif_{C}EloB/C and SOCS2_{SOCS-box}/EloB/C

Starter cultures of BL21(DE3) cells with the requisite plasmids were grown at 37° C. overnight in Luria-Bertani (LB) media in the presence of 100 μg ml⁻¹ carbenicillin. Freshly inoculated, large-scale cultures were then allowed to reach an OD₆₀₀ of 0.5 before induction with 0.001 M IPTG at 30° C. for 4 h. Cells were pelleted by centrifugation (10 min, 2830 rcf), frozen in N₂(I), and stored at −90° C. Thawed cell pellets were resuspended in cell lysis buffer A (CLB-A) [0.40 M NaCl, 0.05 M HEPES (pH 7.4), 0.01 M imidazole (pH 7.6), 0.005 M β-mercaptoethanol] and incubated with lysozyme (2 mg ml⁻¹) for 20 min at 4° C., followed by sonication with a Sonic Dismembrator 60 (Fisher Scientific). Nucleic acid was degraded with 50 μg ml⁻¹ DNase I and RNase A (Sigma) for 20 min at 4° C. The lysate was clarified by centrifugation (25 min, 18K×g, 4° C.) and then placed in a cylindrical chromatography column with 1 ml of packed Ni-NTA resin per 4 g cell pellet, pre-equilibrated in CLB-A; the column contents were subjected to rotary mixing for 2 h at 4° C. Resin-bound protein was washed with 50 ml of CLB-A per 1 ml packed Ni-NTA resin, followed by 4 ml of CLB-A+0.03 M imidazole (pH 7.6) per 1 ml packed resin. Protein was eluted with CLB-A plus 0.24 M imidazole (pH 7.6), buffer-exchanged into CLB-A (BioRad 10 DG desalting column) and concentrated to ˜20 mg ml⁻¹ with a 9K MWCO ultrafiltration centrifugal device (ThermoScientific). The cleavable linker between EloB and Vif or between EloB and SOCS2_{SOCS-box} was removed with ProTEV protease (Promega) at 1 u per mg of protein for 20 h at 4° C. ProTEV protease, cleaved linker, partially cleaved linker complex, and non-cleaved protein complex were removed by rotary mixing with 1 ml packed Ni-NTA resin per 50 mg protein at 4° C. for 2 h. Washing the CLB-A column yielded the linker-removed ternary complex, which was concentrated to 20 mg ml⁻¹. This protein was subjected to size-exclusion chromatography with a Sephacryl S-100 column equilibrated with a buffer comprising 0.125 M NaCl, 0.02 M HEPES (pH 7.4), and 0.005 M β-mercaptoethanol. The eluted protein was concentrated to 3 mg ml⁻¹, frozen as beads in N₂(I), and stored at −90° C. The purity of the respective Vif_{C}/EloB/C and SOCS2_{SOCS-box}/EloB/C were estimated to be >99% by coomassie-stained SDS-PAGE (FIG. S1).

### Cloning, Expression, and Purification of Vif/EloB/C/CBFβ

Expression of the quaternary Vif/EloB/C/CBFβ complex entailed co-expressing EloB₁₋₁₁₈ and EloC₁₇₋₁₁₂ from MCS1 and MCS2, respectively, from a pETDuet-1 vector (pEloBC). Expression vectors were produced as follows. Plasmid pEloBC was constructed from pV_{C}EloBC (described elsewhere herein) by introducing a STOP codon after EloB residue 118 using the Quickchange Lightning site-directed mutagenesis kit (Stratagene). HXB2 Vif₁₋₁₉₂ was cloned into pCDFDuet-1 (EMD4Biosciences) using 5′ Nco1 and 3′ EcoR1 restriction sites to generate pVif. The gene for core-binding factor β subunit isoform 2 (CBFβ) (Open Biosystems) was cloned into MCS1 of pRSFDuet-1 (EMD4Biosciences) using 5′ Nco1 and 3′ EcoR1 restriction sites; primer extension PCR was used to encode an upstream, N-terminal HIS tag and a 3C protease site (MGHHHHHHLEVLFQGP) (SEQ ID NO: 22). This vector was called pCBFβ. All constructs were verified by DNA sequencing.

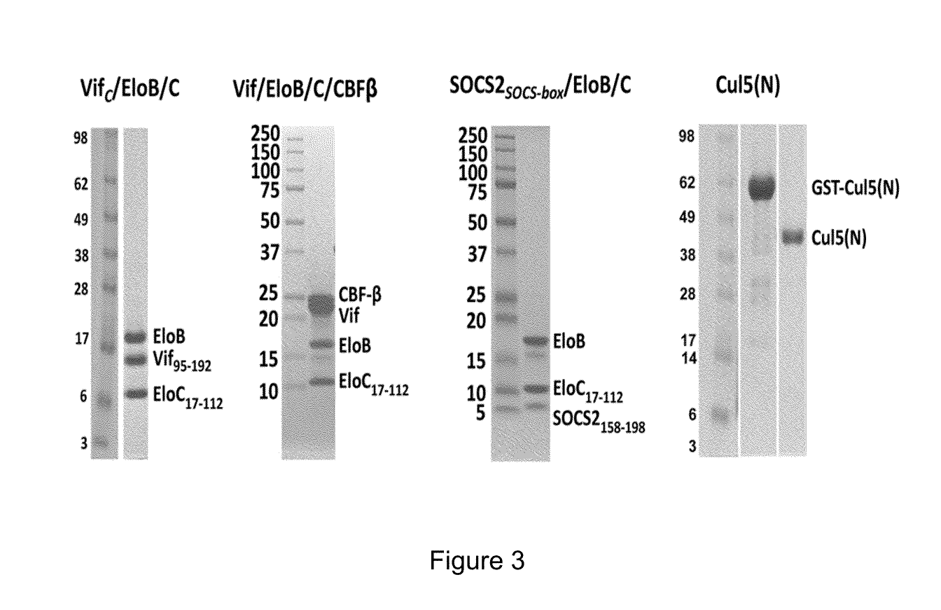

For expression, 20 ng of each vector were co-transformed into competent E. coli BL21(DE3) cells. Cells were grown at 37° C. as overnight starter cultures in LB media containing 30 μg mL⁻¹ of kanamycin, 50 μg mL⁻¹ of spectinomycin, and 50 μg mL⁻¹ carbenicillin. Large-scale flasks with fresh antibiotics were inoculated with overnight growths. When these growths reached an OD₆₀₀ of 0.5, expression was induced by the addition of 0.0003 M IPTG. The cultures were allowed to grow for 20 h at 20° C. Cells were pelleted by centrifugation (10 min, 2.8K×g), frozen in N₂(I), and stored at −90° C. Thawed cell pellets were suspended in cell lysis buffer B (CLB-B) [0.20 M NaCl, 0.05 M Tris-HCl (pH 8.0), 0.02 M imidazole (pH 8.0), and 0.005 M β-mercaptoethanol] and incubated with lysozyme (2 mg mL⁻¹) for 20 min at 4° C., which was followed by sonication with a Sonic Dismembrator 60 (Fisher Scientific). Nucleic acid was degraded with 100 μg mL⁻¹ DNase I and RNase A (Roche) for 20 min at 4° C. Soluble protein was separated from insoluble material and purified by Ni-NTA resin (Qiagen) as described for EloB/C/Vif_{C} (above) except that the protein-bound resin was washed additionally with 10 mL CLB-B+0.02 M imidazole (pH 8.0) and 10 mL of CLB-B+0.05 M imidazole (pH 8.0) per 1 mL of packed resin. The eluted quaternary complex was buffer-exchanged into IEX-1 buffer [same as CLB-B, but without imidazole] as described elsewhere herein (BioRad 10 DG desalting column). Cation exchange chromatography was run on a 5 ml HiTrap FF SP column (GE Healthcare) with a 0.15-1.50 M NaCl gradient in 0.05 M HEPES (pH 7.0), followed by size-exclusion chromatography on a Sephacryl S-300 column (GE Healthcare) equilibrated with buffer comprising 0.15 M NaCl, 0.05 M HEPES (pH 7.0), and 0.005 M β-mercaptoethanol. Eluted protein was concentrated to 30×10⁻⁶ M as described elsewhere herein and stored at −90° C. The quaternary complex purity was estimated at >95% by coomassie-stained SDS-PAGE (FIG. 3).

### Cloning, Expression, and Purification of Human Cul5(N)

To express GST-Cul5₂₋₃₈₄, hereafter referred to as GST-Cul5(N), the Cul5₂₋₃₈₄ DNA sequence (Open Biosystems) was cloned into MCS1 of pRSFDuet-1 (EMD4BioSciences) with 5′ BamHI and 3′ EcoRI restriction sites. The DNA sequence of glutathione-S-transferase (GST), including a C-terminal extension encoding a 3C protease cleavage site, was cloned upstream of the Cul5 sequence using 5′ NcoI and 3′ BamHI restriction sites. To enhance solubility of the truncated Cul5(N) protein, the mutations V341R and L345D were introduced (Zheng et al, 2002, Nature, 416, 703-709) using the Quick Change Lightning Site-Directed Mutagenesis Kit (Stratagene) according to the manufacturer's protocol. These mutations are analogous to those reported for Cullin 1 (Zheng et al., 2002, Nature 416:703-709).

GST-Cul5(N) protein was expressed and cell pellets were lysed as described for Vif_{C}/EloB/C (above) except 60 μg ml⁻¹ kanamycin was substituted for carbenicillin and cell lysis buffer C (CLB-C) [0.20 M NaCl, 0.05 M HEPES (pH 7.4), 0.005 M β-mercaptoethanol] was substituted for CLB-A. Soluble protein was rotated with 1 ml of packed glutathione sepharose 4B resin (GE Healthcare) per 6 g of thawed cell pellet for 2 h at 4° C. Resin-bound protein was washed with 50 ml of CLB-C per 1 ml packed resin. On-resin cleavage was carried out with 10 units PreScission Protease (GE Healthcare) per 1 ml resin at 4° C. for 48 h. Liberated Cul5(N) was recovered by washing the resin with CLB-C, concentrated to 5 mg ml⁻¹, and flash frozen as described elsewhere herein. The purity of Cul5(N) was >98% based on coomassie-stained SDS-PAGE (FIG. 3).

### Thermodynamic Analysis of Protein-Protein Interactions

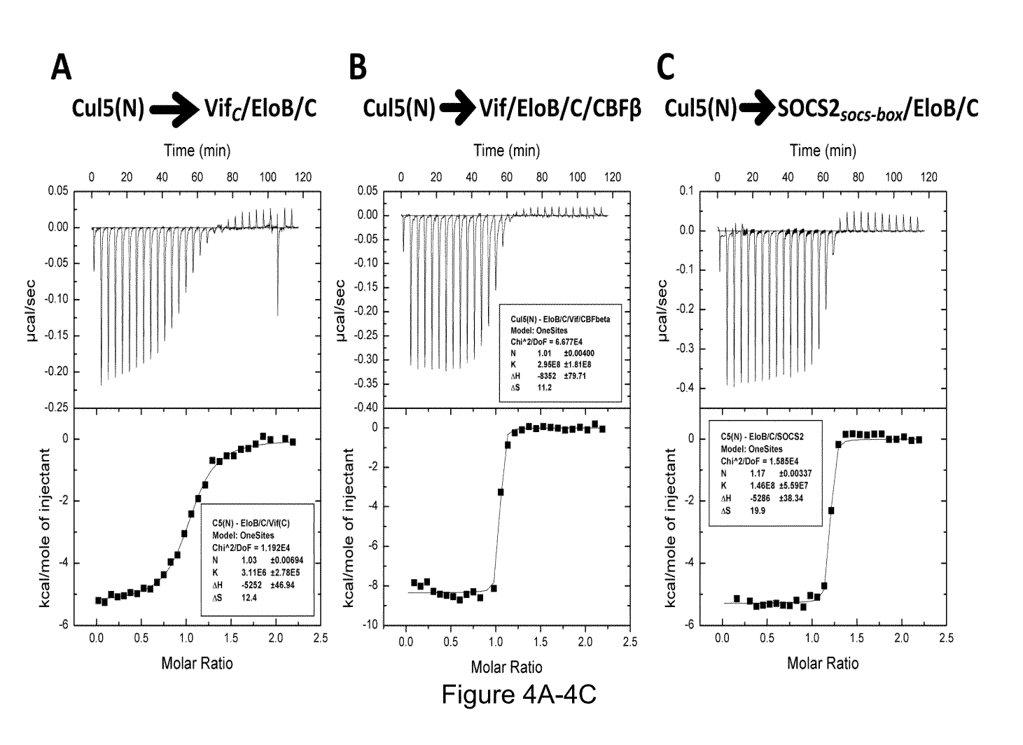

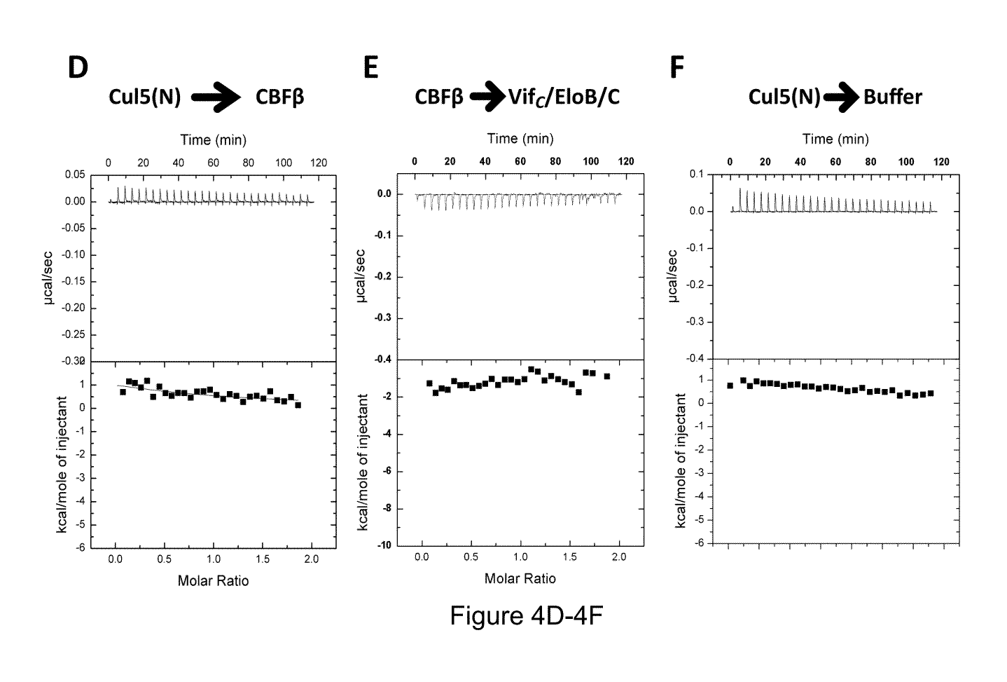

ITC experiments were conducted with a VP-ITC isothermal titration calorimeter (GE Healthcare) at temperatures ranging from 293.15 to 308.15 K using a reference power of 15 μcal s⁻¹ and a 307 rpm stirring rate. Purified protein was dialyzed for 8 h at 4° C. against 1300 volumes of ITC buffer A [0.125 M NaCl, 0.02 M HEPES (pH 7.4), 0.0002 M TCEP] or ITC buffer B [0.125 M NaCl, 0.02 M phosphate (pH 7.4), 0.0002 M TCEP]. Twenty-nine 10 μl aliquots of protein in the syringe were injected into the sample cell at time intervals ranging from 240 to 360 s. The concentration of protein in the syringe ranged from 120 to 210 μM, while the concentration of protein in the sample cell ranged from 12 to 27 μM. Representative isotherms for ITC titrations in ITC Buffer A at 303.15 K are shown in FIG. 4. Errors in the thermodynamic parameters were obtained from the standard deviation resulting from average values with at least two replicates. The titration of Cul5(N) into ITC Buffer A (FIG. 4F) established that the heat of dilution was relatively constant over the course of the entire experiment. The heat of dilution of Cul5(N) into ITC Buffer A was endothermic (average heat of injection=638±185 cal mol⁻¹); thus, upon reaching saturation the heats of injection for titration of Cul5(N) into each protein complex were endothermic. As such, for all protein-into-protein experiments, the heats of injection were accounted for by subtracting the average heat of the last three injections from each data point of an experiment. All data were fit with the “One Set of Sites” curve-fitting model to determine the parameters Kₐ, n, and AH using the Origin software v7.0 (GE Healthcare). ΔG was calculated from ΔG=−RT ln Kₐ, where R=1.9872 cal K⁻¹ mol⁻¹, and T=absolute temperature (Kelvin). ΔS was calculated from the equation, ΔG=ΔH−TΔS. Although high, the C values used here [where C=Kₐ•[cell protein concentration]•n] for titration of Cul5(N) into Vif/EloB/C/CBFβ and Cul5(N) into SOCS2_{SOCS-box}/EloB/C were necessary to obtain sufficient heats of injection. Lower C values resulted in poorer fits of the data. As such, the current equilibrium dissociation values represent the apparent K_{d} (derived from 1/Kₐ), which should be considered the upper limit for the dissociation constant of each interaction shown in FIGS. 4B and 4C, and reported in Table 1.

TABLE 1
Average Thermodynamic Parameters at 303 K for Cul5(N) Binding to Vif/EloB/C/CBFβ

| Syringe | | ΔG | ΔH | −TΔS | K_{d}^{a} | ΔCₚ^{b} | | ΔASA^{c} |
|---|---|---|---|---|---|---|---|---|
| sample | Cell sample | kcal mol⁻¹ | kcal mol⁻¹ | kcal mol⁻¹ | nM | kcal mol⁻¹ K⁻¹ | Nᵣₑₛ^{c} | Å² |
| Cul5(N) | Vif_{C}/EloB/C | −9.0 ± 0.1 | −5.2 ± 0.4 | −3.8 ± 0.5 | 327 ± 40 | −0.30 ± 0.01 | 20 | ~2,100 |
| Cul5(N) | Vif/EloB/C/CBFβ | −11.5 ± 0.3 | −8.8 ± 0.6 | −2.8 ± 0.9 | 5 ± 2 | −0.52 ± 0.02 | 37 | ~3,600 |
| Cul5(N) | SOCS2_{SOCS-box}/EloB/C | −11.3 ± 0.1 | −5.4 ± 0.2 | −5.9 ± 0.0 | 7 ± 2 | N/A | N/A | N/A |

^{a}The C values of 3,200 and 2,783, for titration of Cul5(N) into Vif/EloB/C/CBFβ and SOCS2_{SOCS-box}/EloB/C, respectively, prohibit determination of unique Kₐ values. Thus, the apparent K_{d} values represent lower limits of affinity.
^{b}Heat capacity derived from slope of ΔH vs. T (K) plotted at four temperatures between 293.15 and 308.15 K.
^{c}Number of residues buried (Nᵣₑₛ) and change in solvent-accessible surface area (ΔASA) upon interaction derived from ΔCₚ.

### Measuring the Change in Heat Capacity for Protein-Protein Interactions

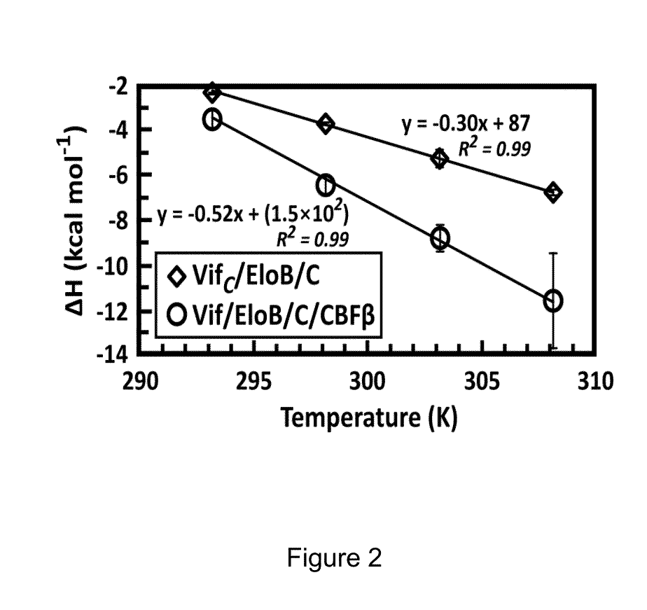

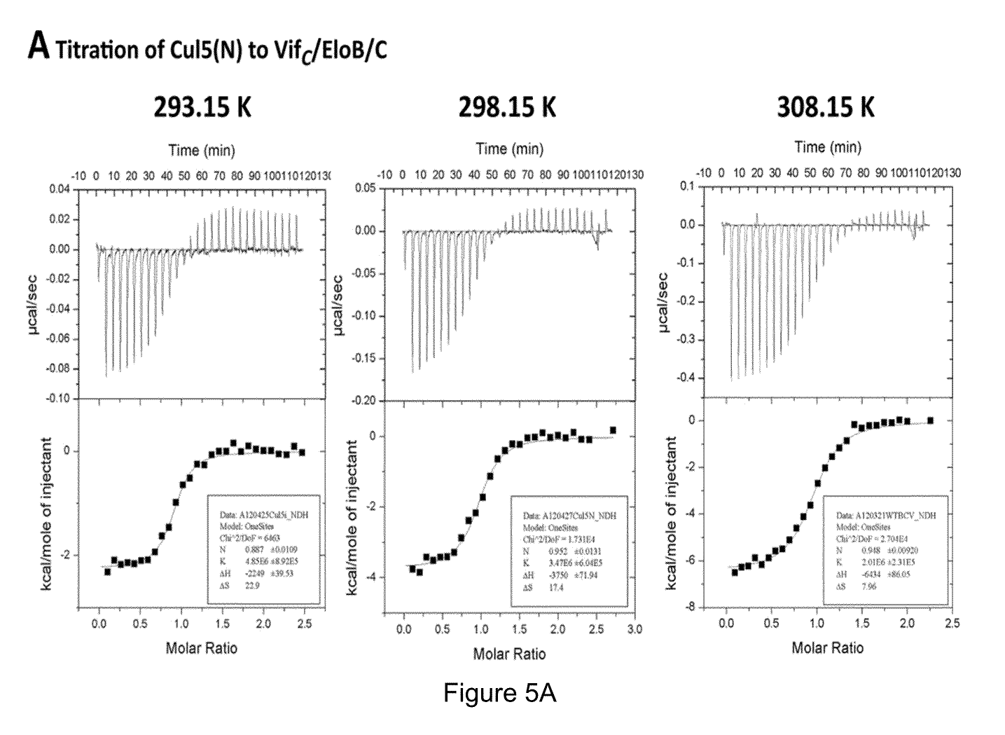

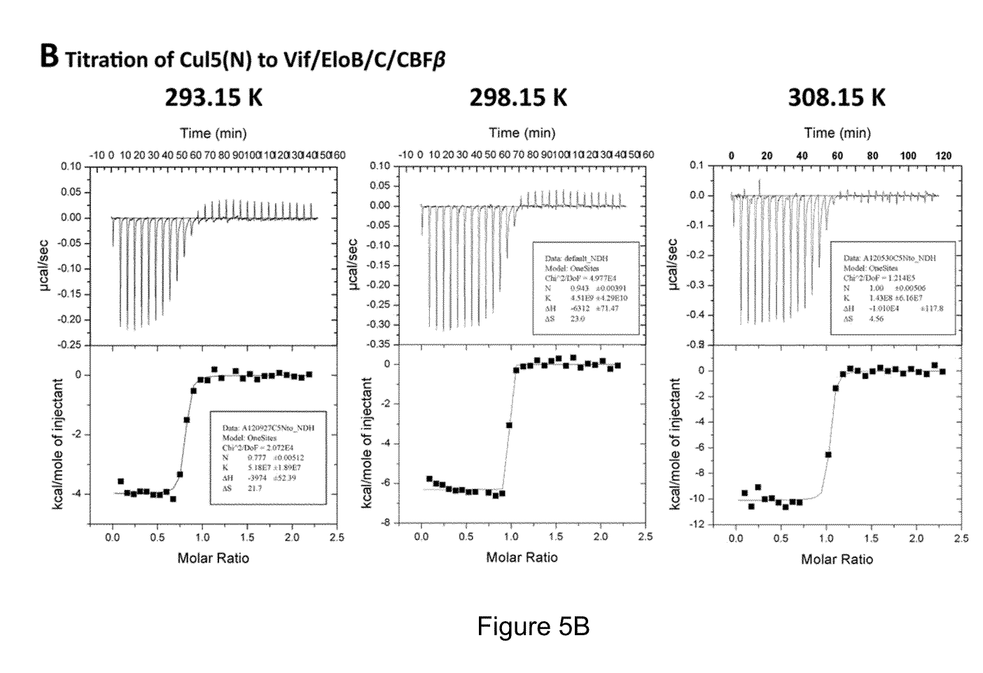

ΔH was measured for the titration of Cul5(N) into Vif_{C}/EloB/C and Cul5(N) into Vif/EloB/C/CBFβ at 293.15, 298.15, 303.15, and 308.15 K in ITC Buffer A (Table 1). Representative isotherms for each interaction at 293.15, 298.15, and 308.15 K are shown in FIG. 5; isotherms at 303.15 K are shown in FIG. 4B and FIG. 4C. The change in heat capacity (ΔCₚ) was determined as the slope of the best-fit line from a plot of 4H vs. T for the respective interactions (FIG. 2). The ΔCₚ was assumed to be constant over the temperature ranges analyzed, which is reasonable based on prior reports (Privalov and Gill, 1988, Adv Protein Chem. 39:191-234). As such, the data were fitted to a straight line using least-squares fitting with Prism v. 6.0 and standard errors were calculated.

### Calculation of Residue Burial and the Change in Solvent-Accessible Surface Area for Cul5 (N) Interaction

An empirical approach relating ΔCₚ of a protein-protein interaction to the number of residues (Nᵣₑₛ) buried upon folding, and the total buried surface area (i.e. change in solvent-accessible surface area, ΔASA) has been described (Robertson and Murphy, 1997, Chem Rev 97:1251-1268). The relationships are: ΔCₚ=(−14)Nᵣₑₛ and ΔCₚ=(−0.146)ΔASA, respectively. For the interaction of Cul5(N) with Vif_{C}/EloB/C, 20 residues in the interface was calculated, with ˜2,100 Å² buried (Table 1), which is typical for a heterodimeric interface (Lo Conte et al., 1999, J Mol Biol 285:2177-2198); while for the interaction of Cul5(N) with Vif/EloB/C/CBFβ, 37 residues were calculated to be buried in the interface with ˜3,600 Å² buried.

### Effect of pH Buffer Composition on ΔH Measurement by ITC

Some protein-protein interactions are associated with side-chain and solvent protonation/deprotonation events. The protonation/deprotonation of a solvated buffer, such as HEPES used here, can contribute an enthalpic component to the total measured ΔH, in accord with its heat capacity (ΔCₚ), thereby obfuscating the actual ΔCₚ for the protein-protein interaction (Baker and Murphy, 1996, Biophysical Journal 71:2049-2055; Leavitt and Freire, 2001, Curr Opin Struct Biol 11:560-566). To assess whether the interaction of Cul5(N) with Vif-containing complexes is accompanied by a protonation/deprotonation event, ΔH was measured for the protein-protein interactions in both ITC buffer A and ITC buffer B. At 303.15 K, HEPES (ITC buffer A) and phosphate (ITC buffer B) have ΔH values for proton dissociation of 5.08 and 1.00 kcal mol⁻¹, respectively; ΔH values were extrapolated from ΔH values calculated at 298.15 K (5.02 kcal mol⁻¹ for HEPES and 1.22 kcal mol⁻¹ for phosphate) using ΔCₚ of 11.7 and −44.7 cal mol⁻¹ K⁻¹ for HEPES and phosphate, respectively (Fukada and Takahashi, 1998, Proteins 33:159-166). If solvent protonation were associated with the protein-protein interaction, the ΔΔH of the interaction in different buffers should be 4.08 kcal mol⁻¹. However, the ΔΔH of the interaction of both Cul5(N) with Vif_{C}/EloB/C and with Vif/EloB/C/CBFβ in the two different buffers is small (Table 2), thereby establishing that protonation/deprotonation events are not likely to contribute to the ΔH values resulting from titration of Cul5(N) into Vif_{C}/EloB/C or Cul5(N) into Vif/EloB/C/CBFβ.

TABLE 2
Effect of pH buffer on ΔH of Cul5(N) interaction with
Vif_{C}/EloB/C and Vif/EloB/C/CBFβ

| | | ΔH | ΔΔH |
|---|---|---|---|
| Titration¹ | Buffer² | (kcal mol⁻¹) | (kcal mol⁻¹) |
| Cul5(N) → Vif_{C}/EloB/C | HEPES | −5.2 | 0.8 |
| | Phosphate | −4.4 | |
| Cul5(N) → Vif/EloB/C/CBFβ | HEPES | −9.2 | 1.7 |
| | Phosphate | −7.5 | |

¹Titrations done at 30° C.
²HEPES present in ITC Buffer A and Phosphate present in ITC Buffer B.

The results of the experiments are now described.

Prior calorimetric analysis showed strong binding of mouse Cul5(N) to Vif(100-192)/EloB/C (K_{d} 89±26 nM) (Wolfe et al., 2010, J Virol 84:7135-7139). Despite the importance of CBFβ in fortifying the interaction between HIV-1 Vif and its host-binding partners (Zhang et al., 2012, Nature 481:376-379; Jager et al., 2012, Nature 481, 371-375), the affinity of Cul5 for Vif/EloB/C/CBFβ has not been quantified. To assess the effect of CBFβ on the interaction between Cul5 and Vif, a thermodynamic analysis was performed, which examined human Cul5(N) binding to: (i) the Vif(95-192)/EloB/C complex, herein called Vif_{C}/EloB/C, where Vif's N-terminal truncation precludes CBFβ binding (Zhang et al., 2012, Nature 481:376-379); and (ii) a complex with full-length forms of Vif and CBFβ, herein called Vif/EloB/C/CBFβ. The resulting parameters were then compared to the Cul5(N) interaction with a minimal human SOCS2/EloB/C complex, which is representative of cellular SOCS-box affinity.

The inability to express Vif as an isolated polypeptide necessitated its production in the presence of its host partners (Barraud et al., 2008, Curr HIV Res 6:91-99). Efforts to produce a Vif/EloB/C complex comprising full-length Vif, but missing CBFβ, were confounded by poor solubility. As such, Vif was expressed in E. coli as Vif_{C}/EloB/C or Vif/EloB/C/CBFβ. Both complexes and Cul5(N) were purified to homogeneity (FIG. 3). Thermodynamic measurements were then conducted for the interaction of Cul5 (N) with ternary and quaternary complexes (FIG. 4A and FIG. 4B).

Results presented herein revealed that human Cul5(N) interacts strongly with Vif_{C}/EloB/C, which harbors the conserved HCCH Zn²⁺-binding motif and the BC-box. The interaction was favorable enthalpically (ΔH=−5.2±0.4 kcal mol⁻¹) and entropically (−TΔS=−3.8±0.5 kcal mol⁻¹) (Table 1) in agreement with results on the closely related mouse Cul5(N) (Wolfe et al., 2010, J Virol 84:7135-7139). Likewise, the interaction between Cul5(N) and the quaternary complex, comprising full-length Vif and CBFβ, was also favorable (ΔH=−8.8±0.6 kcal mol⁻¹) and (−TΔS=−2.8±0.9 kcal mol⁻¹). However, the upper limit of the affinity of Cul5(N) for Vif/EloB/C/CBFβ was 65-fold greater (apparent K_{d}=5±2 nM) than for Vif_{C}/EloB/C (K_{d}=327±40 nM). Cul5(N) affinity in the presence of CBFβ was on par with the SOCS2_{SOCS-box}/EloB/C interaction (apparent K_{d}=8 nM, Table 1 and FIG. 4C), which comprises the human SOCS2 SOCS-box (residues 158-198). The stoichiometry of Cul5(N) binding to each complex was 1:1 (n=0.99 and 0.94, respectively), consistent with its binding to EloB/C bound to cellular SOCS-box proteins (Babon et al., 2009, J Mol Biol 387:162-174).

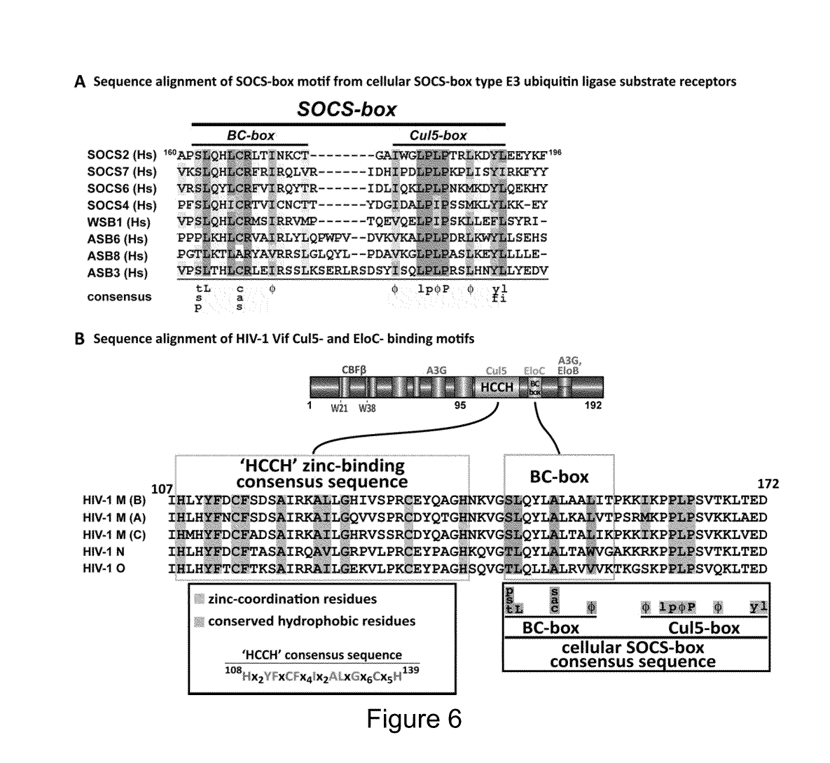

At present, the structural basis of Vif's greater affinity for Cul5(N) in the presence of CBFβ is unknown. The increased affinity of Cul5(N) for Vif/EloB/C/CBFβ over Vif_{C}/EloB/C supports a prior hypothesis that CBFβ acts as a Vif ‘regulator’ that promotes Vif affinity for Cul5 via conformational stabilization (Zhang et al., 2012, Nature 481:376-379; Zhou et al., 2012, PLoS One 7:e33495; Hultquist et al., 2012, J Virol 86:2874-2877; Jager et al., 2012, Nature 481, 371-375.). The results presented herein further support this idea since CBFβ did not interact with Vif_{C}/EloB/C or Cul5(N) alone (FIG. 4D and FIG. 4E) in accord with prior co-immunoprecipitation data (Zhang et al., 2012, Nature 481:376-379). To probe the influence of CBFβ on the Cul5-Vif interaction, ΔCp was measured for Cul5(N) binding to Vif_{C}/EloB/C and Vif/EloB/C/CBFβ, respectively (FIG. 2 and FIG. 5). ΔCp for the interaction of Cul5(N) with VifC/EloB/C and Vif/EloB/C/CBFβ was −0.30±0.01 and −0.52±0.02 kcal K⁻¹ mol⁻¹, respectively. ΔCp<0 can indicate a predominantly apolar interface whereas a ΔCp>0 suggests a predominantly polar one [reviewed in (Janin et al., 2008, Q Rev Biophys 41:133-180; Prabhu and Sharp, 2005, Annu Rev Phys Chem 56:521-548)]. The present findings are consistent with the presence of conserved apolar residues in the Vif HCCH motif reported as crucial for Cul5 binding and HIV-1 infectivity [(Xiao et al., 2006, Virology 349:290-299) and FIG. 6]. Notably, the results presented herein support a direct interaction of Vif residues with Cul5.

Several interpretations are possible for the nearly 2-fold difference in ΔCp for the Cul5(N) interaction with the respective ternary and quaternary Vif complexes in Table 1. Proton transfer effects were ruled out by conducting measurements in buffers with disparate deprotonation enthalpies, which revealed negligible ΔH changes (Table 2). Other possibilities include ion transfer, or protein conformational changes upon complex formation. Notably, large negative ΔCp values—as in Table 1—correlate highly with burial of hydrophobic area (Robertson and Murphy, 1997, Chem Rev 97:1251-1268). As such, an empirical approach was used to estimate the size of binding interfaces. For the Cul5(N) interaction with Vif_{C}/EloB/C, 20 residues were calculated in the interface with ˜2,100 Å² buried (Table 1)—typical of a heterodimeric interface. These values may represent interfaces between Cul5 and the combined surface of Vif's HCCH motif and EloC (Wolfe et al., 2010, J Virol 84:7135-7139; Babon et al., 2009, J Mol Biol 387:162-174). By contrast, Cul5(N)'s interaction with Vif/EloB/C/CBFβ nearly doubles the buried residues to 37 with a buried area of ˜3,600 Å² (Table 1). As a caveat, any values would be misestimated if protein conformational rearrangements accompany binding.

The absence of experimental structures for the Vif complexes in Table 1 leaves the location of putative buried area an open question, especially beyond the well-studied HCCH motif. While not wishing to be bound by any particular theory, possibilities include Cul5(N) interactions with N-terminal regions of Vif, CBFβ, or both. While a direct interaction between Cul5 and CBFβ in the context of EloB/C/Vif/CBFβ cannot be ruled out, it is unprecedented in E3 ligases. By contrast, several conserved N-terminal residues of Vif (⁸⁶SIEW⁸⁹, T⁹⁶, A¹⁰³ and D¹⁰⁴) have been implicated in Cul5 binding (Dang et al., 2010, J Virol 84:8561-8570; Dang et al., 2010, J Virol 84:5741-5750), albeit direct interactions have not been shown. Alternatively, the added buried area could arise from CBFβ's ability to prestabilize Vif's HCCH motif, making it more receptive to subsequent Cul5 binding. In either case, the data presented herein support a prior hypothesis that CBFβ up-regulates Vif's interaction with Cul5 (Zhou et al., 2012, PLoS One 7:e33495), which is akin to its role in promoting α-subunit binding to DNA (Bravo et al., 2001, Nat Struct Biol 8:371-378).

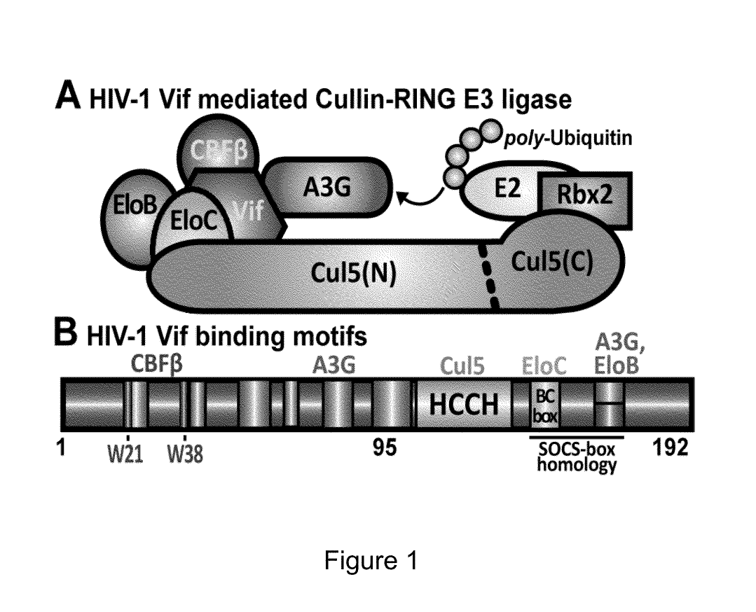

Despite the fact that CBFβ is not required for the Cul5-Vif_{C}/EloB/C interaction, and that Cul5 and CBFβ bind to disparate regions of Vif (FIG. 1B), the present results demonstrate that CBFβ, and the N-terminal half of Vif, enhance the affinity of Cul5(N) for Vif. These factors nearly double the buried area for this host-virus interaction. Importantly, the increased buried area suggests a substantial region of the Vif N-terminus, in addition to the HCCH motif, may become buried upon Cul5 binding. Overall the results presented herein quantify Cul5 affinity and have implications for therapeutics designed to disrupt the Cul5-Vif interface.

The production of these proteins and their validation as a high-affinity interacting complex has paved the way to preparation of a high-throughput assay to identify lead compounds.

The data presented herein allows for development of an in vitro screening to identify small molecule compounds in a high-throughput format that selectively bind to Vif and/or inhibit Vif-dependent interactions between the human antiviral proteins APOBEC3G or A3 related family members and the cellular ubiquitination machinery whose function is co-opted by the virus and whose independent and nonselective targeting would otherwise be toxic to the host cell. The key interaction targeted in the assay is between Cullin5 of the host Cullin-RING ligase complex and Vif, which is included as one of the proteins in the tetramer protein complex EloginB/ElonginC/Vif/CBFβ. Specifically targeting Vif avoids toxic effects to the cell that may arise from targeting the ubiquitination machinery of the host.

The results presented herein demonstrate a solution in the art for assaying for agents that bind to Vif as here-to-fore the methods for purifying sufficient soluble and functional full-length Vif for high throughput screening were not known. The propensity of recombinant Vif to aggregate when expressed alone and during isolation and purification has rendered drug targeting of this essential viral protein impossible. It is shown herein that co-expression of CBFβ allows for expression and purification of full-length, high-affinity Vif protein. Compounds that bind to Vif are candidate antiviral compounds as they may inhibit Vif dependent ubiquitination of APBOEC3G and related family members but also may interfere with Vif binding to APOBEC3G and other A3 proteins or Vif binding to viral capsid proteins and thereby enable increase amounts of APOBEC3G and its family members in order to become incorporated into viral particles leading to the inhibition of viral replication.

The results presented herein demonstrate a solution in the art for assaying for agents that interfere with the binding between Vif and Cul5. The main solution is a method to produce full-length HIV-1 Vif in milligram quantities sufficient for in vitro studies. The results led to the discovery of a method to produce an ElonginB/ElonginC/Vif/CBFβ complex, as well as a region of Cullin5 competent to bind Vif in the context of the former tetramer assembly. The biological significance is that resulting proteins form an interaction network that is necessary and sufficient for probing the Vif-mediated interaction between Cullin5 and ElonginB/C, which is essential to activate the Cullin-RING E3 ligase responsible for degradation of the innate antiviral proteins APOBEC3G and related A3 family members. The mode of generating these proteins in bacteria, their demonstrated solubility, measured high-affinity interactions, and milligram production scale are well suited to development of an assay to screen for small molecule compounds that block the Vif interaction with Cullin5, thus preserving innate antiviral function. The approach disclosed here gets around a standing roadblock in the field in which traditional methods to produce full-length Vif have revealed it is prone to aggregation or insolubility and therefore does not lend itself to in vitro assays required for large-scale, high-throughput screening. While previous methods have been shown to be able to express and purify a C-terminal Vif construct, the present data demonstrates that full-length functionally active Vif is now able to be produced, thereby allowing for interrogation of the entire protein sequence in its ability to bind Cul5.

Protein-protein interactions of Vif are critical for its function as substrate receptor in the E3 ligase complex and HIV-1 infectivity. Thus, blocking one or more of these interactions could abrogate Vif-mediated degradation of A3G, leaving A3G free to carry out its antiretroviral activities. Structure Assisted Drug Design (SADD) involves using pharmacophore maps for screening virtual compound libraries to identify compounds that may prevent Vif protein-protein interactions (Waszkowycz et al., 2001, IBM Systems Journal 40:360-376).

A precedent for using SADD to develop small molecule inhibitors exists and is exemplified well with HIV-1 protease, for which a number of peptidomimetics, such as saquinavir, ritonavir, and indinavir have been designed and act as transition state analogs that bind and block HIV protease activity (Wlodawer, and Vondrasek, 1998, Annu Rev Biophys Biomol Struct 27:249-84). Many other transition state analogs for HIV-1 protease, as well as reverse transcriptase, have been developed with SADD and are in clinical use to treat HIV-1 as a part of HAART (highly active antiretroviral therapy) (Wlodawer, and Vondrasek, 1998, Annu Rev Biophys Biomol Struct 27:249-84).

### Example 2

### Novel Sequences in HIV-1 Vif and Human Cullin5 that Mediate Vif-Cullin5 Binding

Described herein is the discovery of novel sequences in HIV-1 Vif and human Cullin5 (Cul5) that have not been previously described but are now shown to be implicated in forming portions of the molecular interaction interface between Cul5 and the essential HIV-1 protein Vif. As discussed elsewhere herein, Vif is embedded as an integral, full-length sequence component of a multi-protein complex comprising ElonginB/ElonginC/Vif/CBFβ, which is part of a larger human host Cullin-RING E3 ubiquitin-ligase complex whose biological role is to degrade innate immune factors of the host such as APOBEC3G. Knowledge of unique interacting peptides between the host and virus provides a significant advantage in efforts to develop peptide-like molecules intended to disrupt protein interfaces that are essential for viral infectivity.

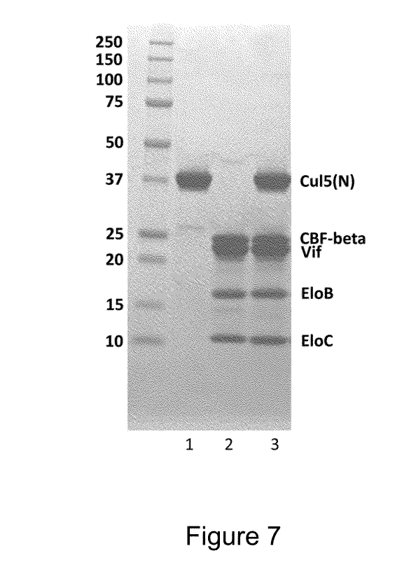

The HIV-1 Vif residues that undergo dynamic exchange upon binding Cul5 have been discovered by Hydrogen Deuterium Exchange Mass Spectrometry (HDX-MS). Milligram quantities of the ElonginB/ElonginC/Vif/CBFβ complex and Cul5(N) have been purified, as described elsewhere herein. The ElonginB/ElonginC/Vif/CBFβ complex was expressed in E. coli and purified using a 6HIS C-terminal tag on CBFβ, followed by SP cation-exchange chromatography and size-exclusion chromatography (FIG. 7). The N-terminal residues (2-384) of Cullin 5 were expressed in E. coli as an N-terminal fusion of GST and purified using glutathione resin followed by cleavage of the GST tag with PreScission Protease (GE), followed by size exclusion chromatography (FIG. 7). A 1:1:1:1:1 stoichiometric pentameric complex comprising ElonginB/ElonginC/Vif/CBFβ/Cul5 was formed by mixing a stoichiometric excess of ElonginB/ElonginC/Vif/CBFβ with Cul5 and pooling the appropriate fractions from a size-exclusion chromatography separation (FIG. 7). Additionally, the complexes formed herein indicate binding activity as expected (including the binding of Cul5 to the ElonginB/ElonginC/Vif/CBFβ complex) and are on par with that of cellular SOCS-box proteins. The details of these interactions are described elsewhere herein. Purified tetramer, Cul5(N), and pentamer were frozen in N₂(1) and sent to ExSAR (New Jersey) for HDX-MS analysis.

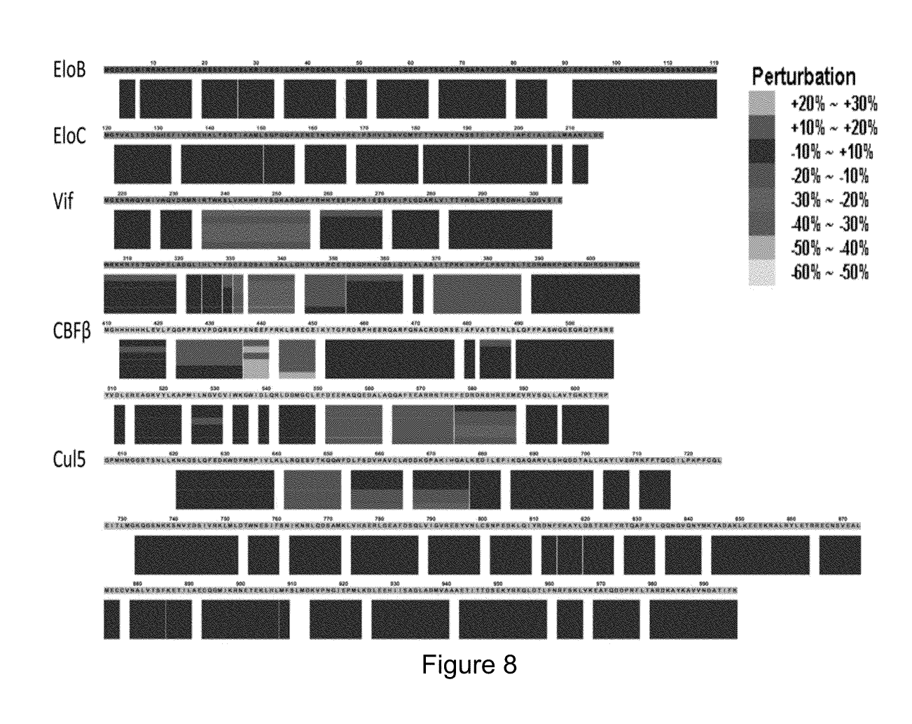

The approach used herein was to use hydrogen-deuterium exchange mass spectrometry (HDX-MS), which was conducted on: (i) the ElonginB/ElonginC/Vif/CBFβ tetramer complex, (ii) Cullin 5, and (iii) the ElonginB/ElonginC/Vif/CBFβ/Cullin 5 pentameric complex, respectively. Each sample was diluted to ˜9 μM in sample buffer, [150 mM NaCl, 20 mM HEPES (pH 7.0)], comprising 60% D₂O for either 15, 50, 150, 500, 1500, or 5000 seconds at 0° C. Hydrogen-deuterium exchange was quenched by dilution with 1.6 M GuHCl and 0.8% formic acid (pH 2.3). Samples were then digested with an immobilized pepsin column; peptides were separated with a reverse-phase C18 column with a linear gradient of 13-35% Buffer B (95% acetonitrile, 5% H₂O, 0.0025% TFA. Peptic fragments from each sample were analyzed by electrospray ioninzation mass spectrometry. Deuterium perturbation analysis of the peptic fragments from the unbound samples (tetramer and Cul5) versus the bound sample (pentamer) identified regions within Vif and Cullin5 that were protected from deuteration as a result of Cullin5 binding to the tetramer. The results reveal five separate segments of Vif and 3 segments of Cul5 (FIG. 8) were significantly protected from hydrogen-deuterium exchange in the bound (pentameric) form compared to the unbound forms (tetrameric and Cul5(N) alone) (FIG. 8). The strong protection from deuteration in the bound form suggests that residues within these regions of Vif and Cul5 are involved in forming a molecular interface during Cul5 binding. Notably, ElonginB and ElonginC did not experience perturbation of deuteration levels upon pentamer formation. The average sequence coverage of all peptides was 95%. Using HDX-MS, specific sequences within HIV-1 Vif (subtype HXB2) were identified including: (a) ¹⁹RTWKSLVKHHMYVSGKARGWF³⁹ (SEQ ID NO: 1), (b) ⁸⁹WRKKRYSTQVDPEL¹⁰⁴ (SEQ ID NO: 2), (c) ¹¹³FD¹¹⁴ (SEQ ID NO: 3), (d) ¹¹⁵CF¹¹⁶ (SEQ ID NO: 4), (e) ¹¹⁷DSAIRKALL¹²⁵ (SEQ ID NO: 5), (f) ¹²⁸IVSPRCEY¹³⁵ (SEQ ID NO: 6), (g) ¹³⁶QAGHNKVGSLQ¹⁴⁶ (SEQ ID NO: 7), and (h) ¹⁵³LITPKKIKPPLPSPTKL¹⁶⁹ (SEQ ID NO: 8).

Exchange perturbed sequences identified within human Cullin5 include: (A) ³⁰LRQESVTKQQW⁴⁰ (SEQ ID NO: 9), (B) ⁴³LFSDVHAVCL⁵² (SEQ ID NO: 10), and (C) ⁵⁵DKGPAKIHQAL⁶⁵ (SEQ ID NO: 11). The data presented herein represents the most complete sequence mapping analysis of this pentapartite complex ever conducted. The identified sequences are not evident or obvious based on current state-of-the field information.

The results suggests that some, or all of the residues within these regions are critical for formation of a biologically functional ElonginB/ElonginC/Vif/CBFβ/Cullin5 complex that forms the core of a host cell's E3 ubiquitin-ligase machine that is required for poly-ubiquitination and subsequent proteasomal degradation of endogenous antiretroviral proteins of the APOBEC3 family.

Currently, there are no high-resolution structural models for the Vif/Cul5 interface. The application of HDX-MS used in the present study to examine this interface—in the context of the very recently identified CBFβ protein—provided a means to identify residues within the host-virus interface. This information provides the identity of peptides that may be operative in disruption of the host-virus protein interaction, and can be developed into a new class of anti-retrovirals against HIV-1.

### Example 3

### Binding of Peptide Inhibitors to Vif/EloB/C/CBFβ

As described elsewhere herein, peptide sequences (SEQ ID NOs: 9-11) were identified that appear to be present in the interface between human Cul5(N) and the HIV-1 Vif/EloB/C/CBFβ complex. Using these results, specific peptides were designed and studied for the ability to bind to the isolated HIV-1 Vif/EloB/C/CBFβ complex. The data presented herein demonstrates the construction of a “lead platform” for the generation of peptides or derivatives thereof that will block the HIV-1 Vif interaction with the host, thereby eliminating an essential viral interaction.

As described elsewhere herein, the following sequences of Cul5 were identified by HDX-MS to participate in the binding between Vif and Cul5: (A) ³⁰LRQESVTKQQW⁴⁰ (SEQ ID NO: 9), (B) ⁴³LFSDVHAVCL⁵² (SEQ ID NO: 10), and (C) ⁵⁵DKGPAKIHQAL⁶⁵ (SEQ ID NO: 11). Using this information, peptides that comprise one or more regions of A, B, or C, or fragments thereof were generated and studied.

The peptides that were studied are depicted in FIG. 9. The peptides include: Cul5(30-65)(SEQ ID NO: 12), Cul5(30-40)(SEQ ID NO: 13), Cul5(43-54)(SEQ ID NO: 14), Cul5(55-65)(SEQ ID NO: 15), Cul5(43-65)(SEQ ID NO: 16), Cul5(37-53)(SEQ ID NO: 17), and Cul5(37-48)(SEQ ID NO: 18).

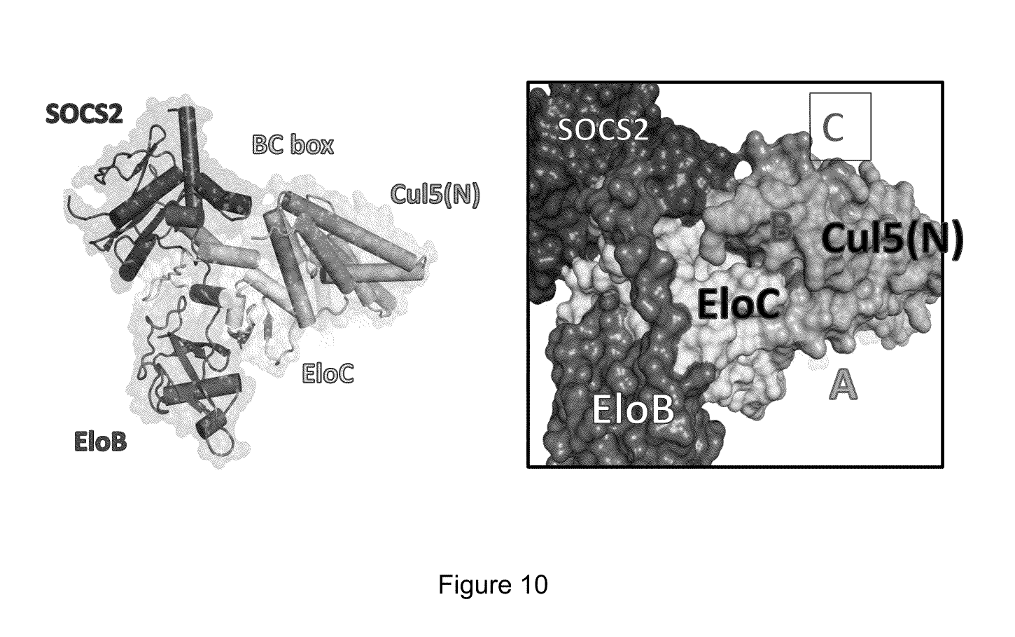

Peptides were mapped onto a recent crystal structure of EloB/C/SOCS2/Cul5(N) (Kim et al., 2013, Acta Crystallogr Sect D, 69: 1587-1597). Structural mapping suggests that a small helical portion of HDXMS region A and most of B form an interface with EloC (FIG. 10). Other regions in A, as well as C, were not expected to show protection from deuteration. This implies that residues within A and C are involved in direct contacts with one of the proteins of the Vif/EloB/C/CBFβ quaternary complex. Alternatively, Cul5(N) adopts a different conformation in isolation than the Cul5(N)/SOC2/EloB/C complex, although superposition of bound and free Cul5(N) structures excludes that latter possibility.

Structural mapping of the Cul5(N) peptides onto the Cul5(N)/SOCS2/B/C crystal structure by Kim et al. revealed that segment A is in an exposed loop with some EloC contacts, region B is mostly buried in the EloC/Cul5 interface, and region C is located in an exposed loop-helix segment (FIG. 10). These findings suggest that HIV-1 Vif utilizes the cellular BC-box as observed, while making unique interactions to Cul5 that include amino acids in regions A and C. As such, peptides comprising sequences from regions A and C have therapeutic potential. While not wishing to be bound by any particular theory, peptides comprising regions A and C may be more specific to inhibiting the binding of Cul5 to Vif, while not effecting physiologically relevant binding to SOCS2.

Based on HDX mass spectrometry experiments synthetic peptides of the N-terminus of Cullin5 were ordered from a commercial vendor (Genscript). Each peptide was N-terminally tagged with a derivative of fluorescein called 5-FAM by an Ahx (aminohexanoic acid) linker.

Each peptide possessed a different charge and therefore was diluted in different solvents prior to anisotropy experiments. Table 3 summarizes the solutions that peptides were resuspended in:

TABLE 3
Cul5 derived synthetic peptides were diluted in the
solvents listed for anisotropy experiments.

| Peptide | Charge | Solvent |
|---|---|---|
| Cul5(30-40) | 0 | NMP* |
| Cul5(43-54) | −2 | 10% NH₄OH |
| Cul5(55-65) | 1 | H₂O |
| Cul5(43-65) | 0 | 10% NH₄OH |
| Cul5(37-48) | −1 | NMP* |
| Cul5(37-53) | −1 | 10% NH₄OH |

*NMP is N-methyl-2-pyrrolidone

Initial peptides in powder form were dissolved in small volumes (50 or 100 μl) of appropriate solvent (Table 3). Dilutions were then made to produce 100 and then 1 μM peptide solutions by use of an empirically developed experimental buffer (henceforth referred to as FA buffer) that comprises: 0.20 M NaCl, 0.05 M HEPES pH 7.4, 0.2 mM TCEP and 0.05 mM Brij 35. The 1 μM peptide solutions were then diluted further with FA buffer to give 25 nM for fluorescence anisotropy measurements.

Fluorescence anisotropy was conducted by titrating concentrated protein (also in FA buffer) into 500 μl of various peptide solutions at 25 nM concentration. The anisotropy readings were measured based on the fluorescence intensity of 5-FAM. The amounts of titrant were increased after each reading on a Fluoromax-3 in order to create binding data fit by a non-linear regression analysis (anisotropy versus concentration of protein titrated). Fits were conducted using Prism software from Graphpad, Inc. The formula that was used for fitting the curve was derived previously as:

Y=Rf+(Rb−Rf)*(0.5/[Peptide])*((Kd+X+[Peptide])−sqrt((Kd+X+[Peptide])̂2−(4*[Peptide]*X)))

where:

X is total protein concentration

Rf is the anisotropy at zero protein concentration

Rb is the anisotropy at saturating protein concentration (floated in fit)

K_{d} is apparent dissociation constant (in the calculations it was set to 1.0 μm)

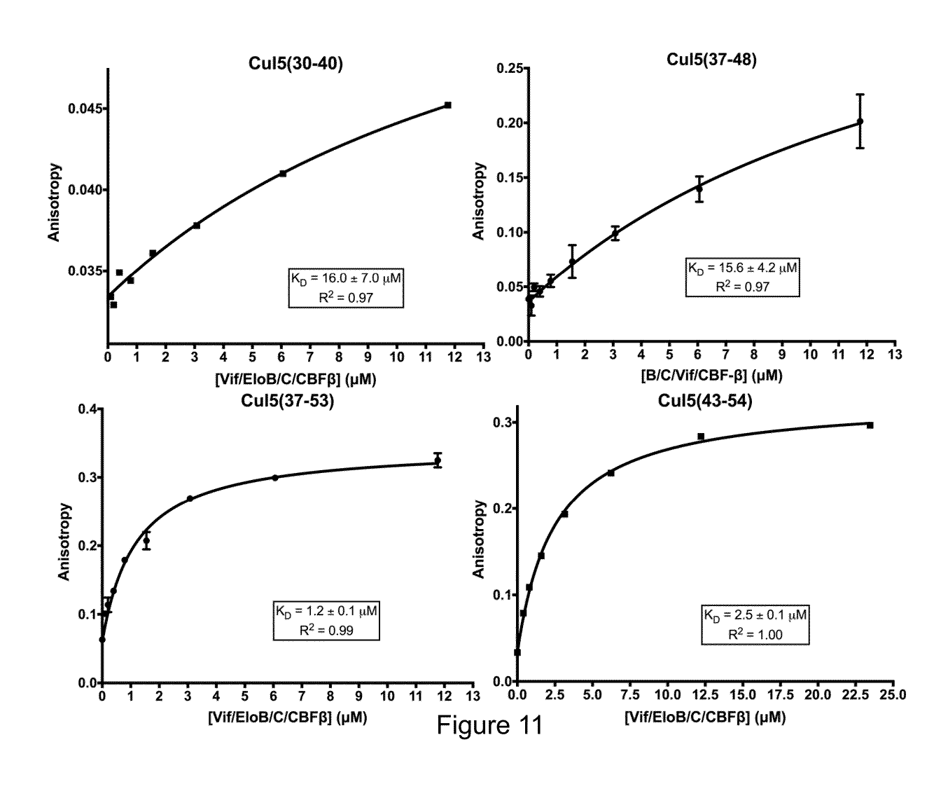

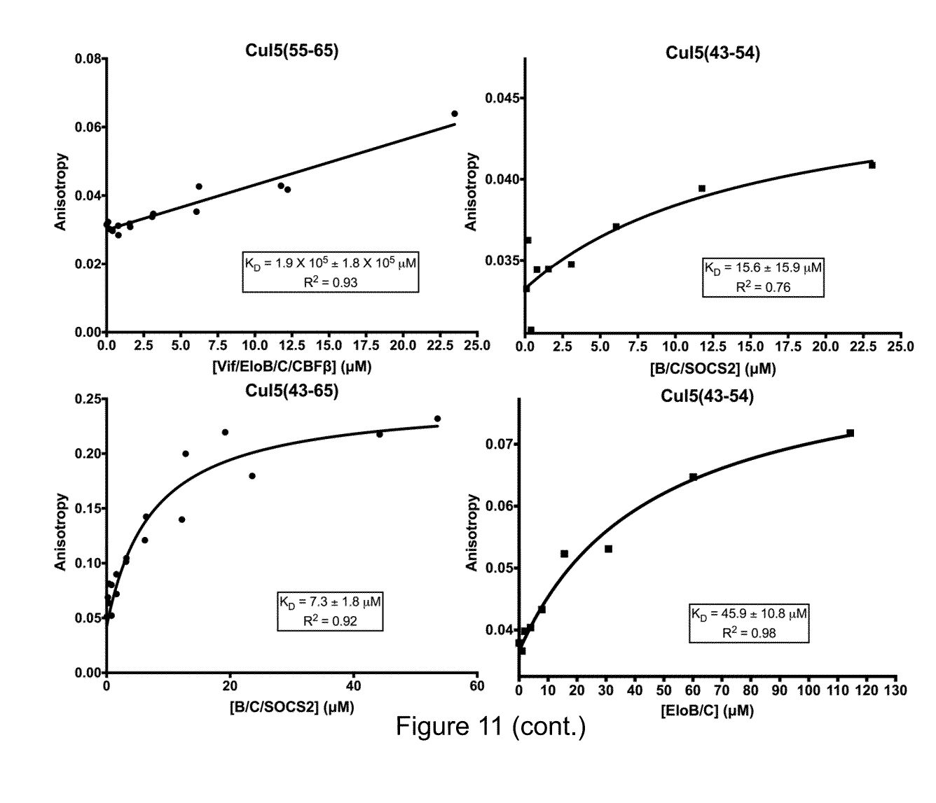

The peptides were examined using fluorescence anisotropy to evaluate binding to isolated Vif/EloB/C/CBFβ, as well as to controls. FIG. 11 depicts the data for the binding of each peptide to either isolated Vif/EloB/C/CBFβ, EloB/C/SOCS2, or EloB/C. The dissociation constant (K_{D}) for each interaction is provided. A summary of the data from the fluorescence anisotropy studies is provided in Table 4.

This data demonstrates that the Cul5 derived peptides examined bind to the Vif/EloB/C/CBFβ complex, and can thus be used as a peptide inhibitor of the interaction of Cul5 with the Vif/EloB/C/CBFβ. Thus, these peptides inhibitors represent therapeutic peptides that can be used to treat viral infections.

TABLE 4
Fluorescence anisotropy data identifying the binding kinetics
of examined Cul5 derived peptides to Vif/EloB/C/CBFβ.

| Complex | Peptide | Kd (μM) | Sd± | R squared |
|---|---|---|---|---|
| Vif/B/C/CBFβ | 30-40 | 16.0 | 7.0 | 0.97 |
| Vif/B/C/CBFβ | 43-54 | 2.5 | 0.1 | 1.00 |
| Vif/B/C/CBFβ | 43-65 | 4.7 | 1.0 | 0.95 |
| Vif/B/C/CBFβ | 55-65 | 190000.0 | 180000.0 | 0.93 |
| Vif/B/C/CBFβ | 37-48 | 15.6 | 4.2 | 0.97 |
| Vif/B/C/CBFβ | 37-53 | 1.2 | 0.1 | 0.99 |
| SOCS2/B/C [(−) control] | 43-65 | 7.3 | 1.8 | 0.92 |
| EloB/C [(−) control] | 43-54 | 45.2 | 10.8 | 0.98 |

### Example 4

### FqRET Assay for High-Throughput Screening

The next set of experiments was designed to provide a method for interrogating the integrity of the Vif/Cul5 interface using a quenched FRET (FqRET) system. This system is used to identify novel compounds (small molecules, peptides or peptidomimetics) that disrupt the aforementioned protein-protein interface. The resulting identified compounds may then be used as lead compounds for antiviral therapeutic development. The mechanism of action is anticipated to be inhibition of HIV-1 infectivity via protection of host-defense factors of the APOBEC3 family (such as APOBEC3G and APOBEC3F) that are susceptible to Vif-dependent degradation, which depends upon the association of Vif with human ElonginB/C and Cullin 5 as a preface to polyubiquitination. The assay is based on a recombinantly produced complex of ElonginB/ElonginC/Vif/CBFβ and a recombinantly produced Cullin5 assembled in vitro. Selected proteins have appropriately positioned FRET donor and FRET quencher molecules linked to the interacting proteins such that a normal, wildtype interaction elicits a dark, quenched fluorescence signal (i.e. FqRET). By contrast, molecules that disrupt the complex are expected to elicit a strong fluorescent signal.

In the context of the present system, the assay works as follows. The expression of a recombinant complex comprising full-length HXB2 Vif (residues 1-192) is predicated upon the inclusion of the recently discovered CBFβ protein. It is discovered herein that co-expression of Vif with CBFβ enables production of: (i) large quantities of soluble Vif, (ii) a Vif protein that is far more functional in terms of binding to Cullin 5 than previously reported, and (iii) this functional form of full-length Vif can be expressed and purified at a modest expense from E. coli bacteria. The assay has been designed to achieve FqRET upon complex formation between ElonginB-EGFP/ElonginC/Vif/CBFβ and Cullin5-REACH2, wherein ElonginB-EGFP fusion protein is the fluorescence donor and Cullin5-REACH2 fusion protein is the fluorescence acceptor and quencher. Quenched complexes are assembled in vitro and dispersed into microtiter plates to retain a low level of fluorescence. When a compound is added during the high-throughput screen that induces the dissociation of Cullin5-REACH2 from the ElonginB-EGFP/ElonginC/Vif/CBFβ complex, a significant fluorescent signal is produced. Other ways of constructing the FqRET system can also be conceived. It is possible to change which terminus of Cullin5 is fused with REACH2. It is also possible to change which terminus of ElonginB is fused with EGFP or switch the EGFP fusion to one of the other proteins of the ElonginB/ElonginC/Vif/CBFβ complex to enhance the FqRET signal.

Such compounds would be considered “hits”. The mode of action by which such hits reverse quenching may be through chemistries that interact with one of the components of the pentapartite complex causing inhibition or competition with the Vif interaction with Cullin 5. These hits are anticipated to have antiviral activity because they will prevent Vif-APOBEC3 family members from interacting with Cullin 5, thereby blocking APOBEC3 from being targeted by the ubiquitination machinery that leads to subsequent proteasomal degradation. Consequently APOBEC3 family members will not be destroyed by Vif and the increased intracellular abundance of these host-defense factors will enable them to enter nascent viral particles from which point the host-defense factors will be positioned to interact with viral replication complexes following infection and thereby block viral infectivity. The interaction of APOBEC3G and Vif is thought to alter APOBEC3G's deaminase processivity but it does not inhibit it nor does it prevent APOBEC3G entry into the budding virus.

FqRET complexes can also be produced by in vitro chemical conjugation of Cullin 5 with commercially available fluorescence donors. Similar conjugation could be conducted on the ElonginB/ElonginC/Vif/CBFβ complex using commercially available fluorescence quenchers. FqRET assays based on complexes composed of proteins chemically conjugated with donor and quencher have greater differentials between quenched and unquenched signals compared to what can be observed in complexes containing EGFP and REACh2 fusion proteins. This is because chemical conjugation positions multiple donors and quenchers along the coupled protein based on the availability of primary and secondary amine groups.

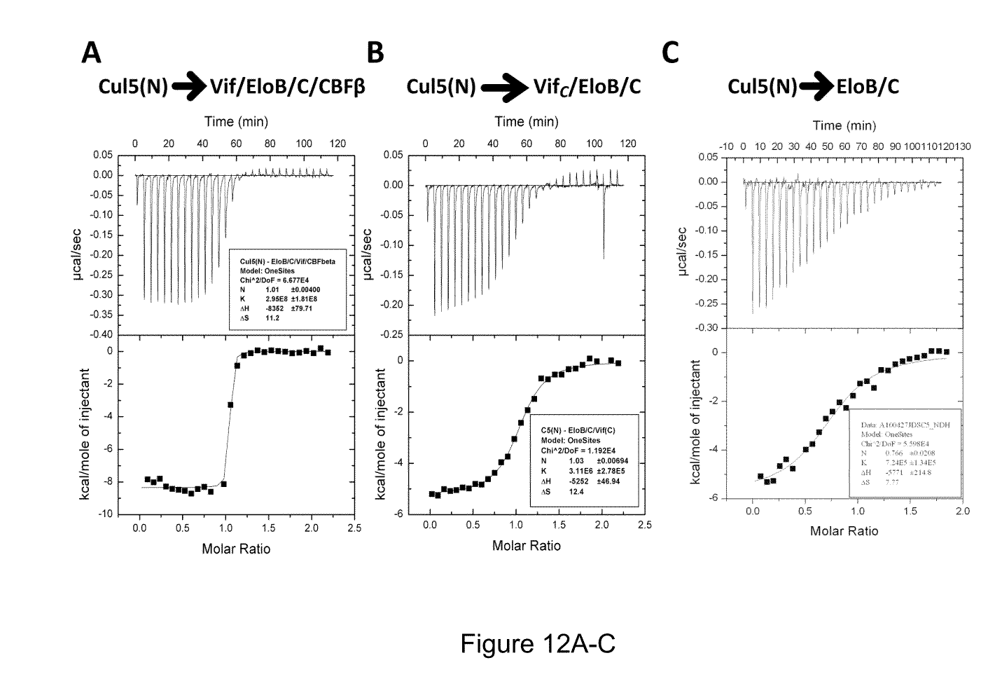

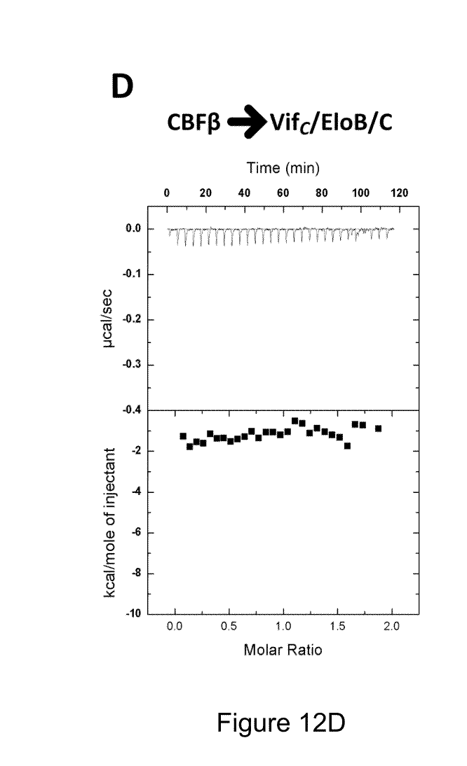

The solubility and functionality of recombinantly expressed ElonginB/ElonginC/Vif/CBFβ and Cullin 5 has been examined. Isothermal titration calorimetry verified the functionality of the tetrameric complex. The ElonginB/ElonginC/Vif/CBFβ complex binds Cul5(N) robustly (FIG. 12A, K_{d} of ˜5±2 nM) while an ElonginB/ElonginC/Vif complex that only comprises the C-terminal half of Vif (95-192) binds ˜65 fold weaker (FIG. 12B, K_{d}=327±40 nM). By contrast, an ElonginB/ElonginC heterodimer bind Cul5 very weakly (FIG. 12C, K_{d}˜6 uM). A direct interaction between CBFβ and Vif's N-terminal half is supported by the finding that CBFβ does not interact with the EloB/EloC/Vif(C-terminal half) complex (FIG. 12D).

An E. coli expression plasmid has been constructed using a pETDuet parent vector (EMD4Bioscience) harboring the genes for expression of both ElonginB-EGFP and ElonginC from two independent cloning sites. Gene sequences have been verified by DNA sequencing. Likewise, a plasmid has been constructed for expression of a Cul5-REACH2 fusion protein comprising the N-terminal 2-384 residues of Cul5 (pRSFDuet, EMD4Bioscience).

A robust Förster quenched-resonance energy transfer (FqRET) assay has been established for high-throughput compound library screening in microtiter plates. The assay is based on selective placement of chromoproteins or chromophores that allow reporting on complex formation between the ElonginB/ElonginC/Vif/CBFβ tetramer and Cul5 in vitro. For example, an appropriately positioned FRET donor and FRET quencher results in a “dark” signal when the pentameric complex is formed. The assay is designed to achieve FqRET based on formation of a complex between ElonginB/ElonginC/Vif/CBFβ and Cul5 in which the latter is expressed as Cullin5-REACH2 fusion protein as the fluorescence acceptor and quencher, and the former is expressed as an ElonginB-EGFP/ElonginC/Vif/CBFβ protein, where ElonginB-EGFP is the fluorescence donor. Quenched complexes assembled in vitro and dispensed into microtiter plates will retain low levels of fluorescence. When the appropriate compound has been identified to block the Vif-Cul5 interaction, the complex disassociates leading to a fluorescence signal. Without wishing to be bound by any particular theory, it is believed that the use of small molecule chromophores to tag the respective proteins as well, which should elicit the same response FqRET response.

Based on the disclosure presented herein, the design of an FqRET assay for high-throughput screening of inhibitors of Vif-mediated binding of Cullin 5 to the ElonginB/C complex can be adapted to any appropriate chemical conjugation of fluorescence donors and quenchers to interacting proteins involved in complex formation.

The assay described herein allows for the identification of ‘hit’ small molecules (organic compounds, peptides or peptidomimetics) that reduce the FqRET signal by disrupting the Vif-Cullin5 interface. In addition, the assay is amenable to screening molecules and lead compounds for optimal disruption properties for the Vif-Cullin5 interface in live cells.

### Example 5

### Expression and Quenching of Tagged FqRET Assay Components

Experiments were conducted to examine the functionality of the FqRET based assay described herein. For example, experiments presented herein are used to identify an optimal configuration of labeled Vif/EloB/C/CBFβ and labeled Cullin5 for use in the assay.

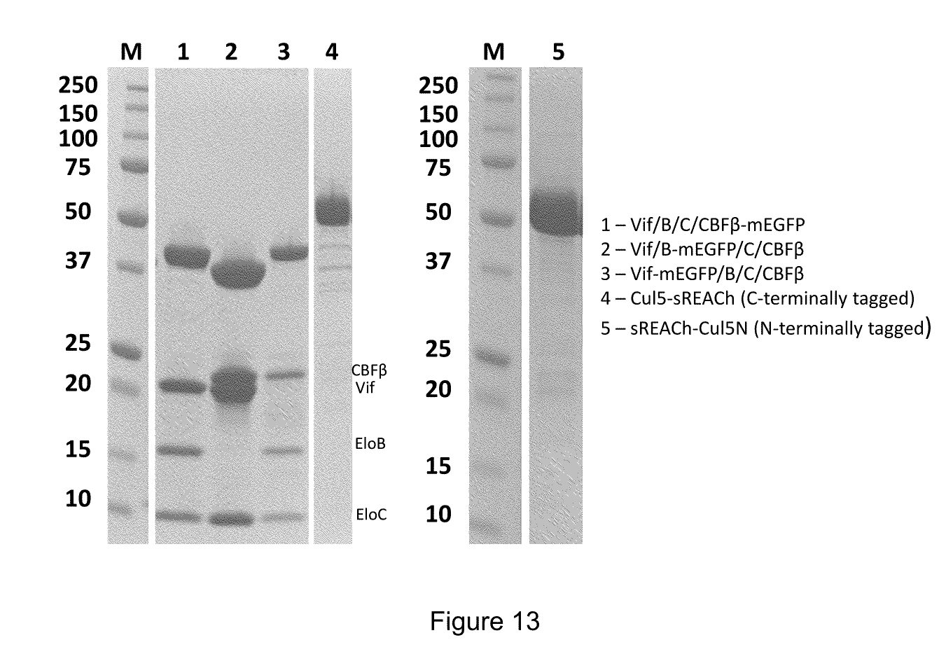

Various complexes were produced using fluorescently tagged proteins in order to identify the optimal FRET quenching combination. Co-expression was optimized using a pCDFDuet-1 plasmid containing full-length Vif(1-192)-mEGFP in multiple cloning site 1 (MCS1), pETDuet-1 with ElonginB-mEGFP and ElonginC in MCS1 and 2, respectively, and 6His-CBFβ(1-182)-mEGFP in MCS1 of pRSFDuet-1. All three plasmids have different ORFs and antibiotic-resistance markers, thus allowing them to be co-expressed in a BL21(DE3) cell line. Purification of quaternary complexes was achieved by IMAC, ion exchange, and size exclusion chromatography discussed elsewhere herein. Cul5(2-384)-sREACh and sREACh-Cul5(2-384) proteins were expressed separately as a GST-fusion or 6His-tagged constructs, respectively. The constructs were expressed and the tagged assay proteins were purified using size exclusion chromatography (FIG. 13).

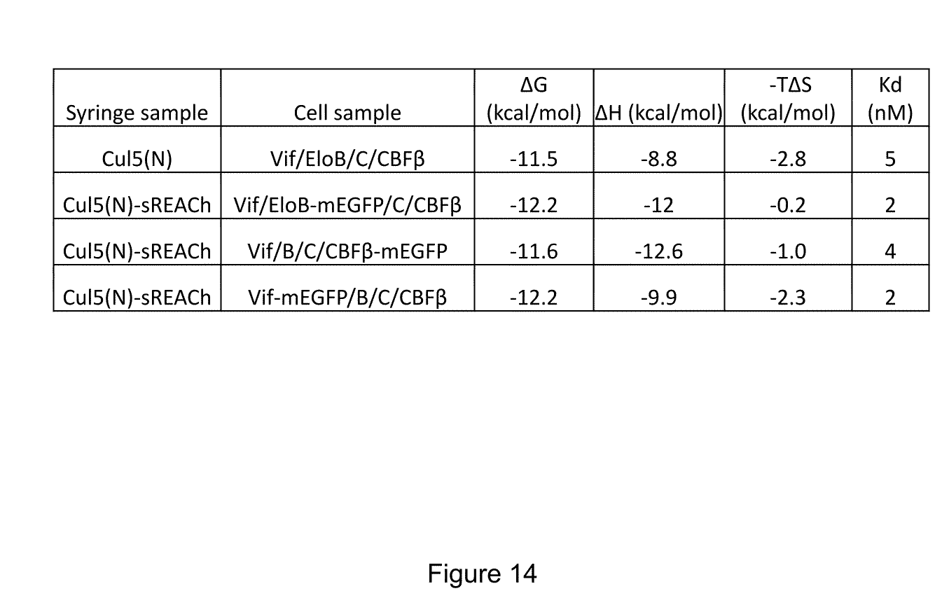

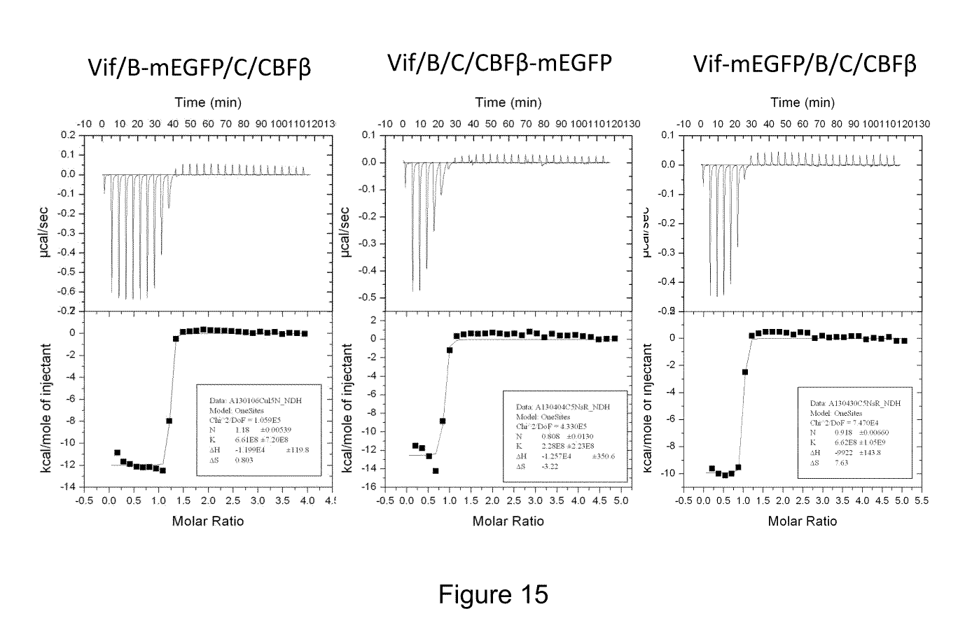

The affinity of Cul5(N) for the tetramer complex was previously benchmarked as reported elsewhere herein. It is demonstrated herein that the interaction between the constructs harboring fluorescent proteins is equally avid. As previously, ITC analysis was used in which Cul5(N)-sREACh was titrated into various quaternary complexes tagged with mEGFP. The observed binding was similar to that between wild-type proteins (FIG. 14 and FIG. 15).

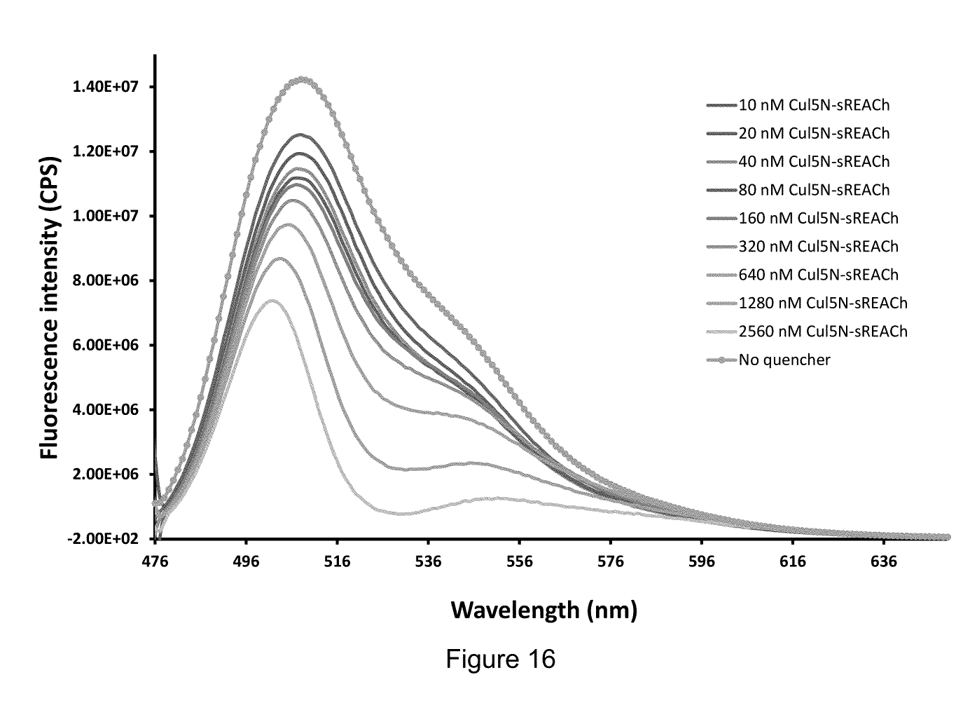

Experiments were performed to investigate the quench between fluorescently tagged quaternary complexes and Cul5(N)-sREACh. FIG. 16 depicts the quenching of Vif/EloB-mEGFP/EloC/CBFβ at 508 nm, when excited at 469 nm, upon the addition of various concentrations of Cul5(N)-sREACh.

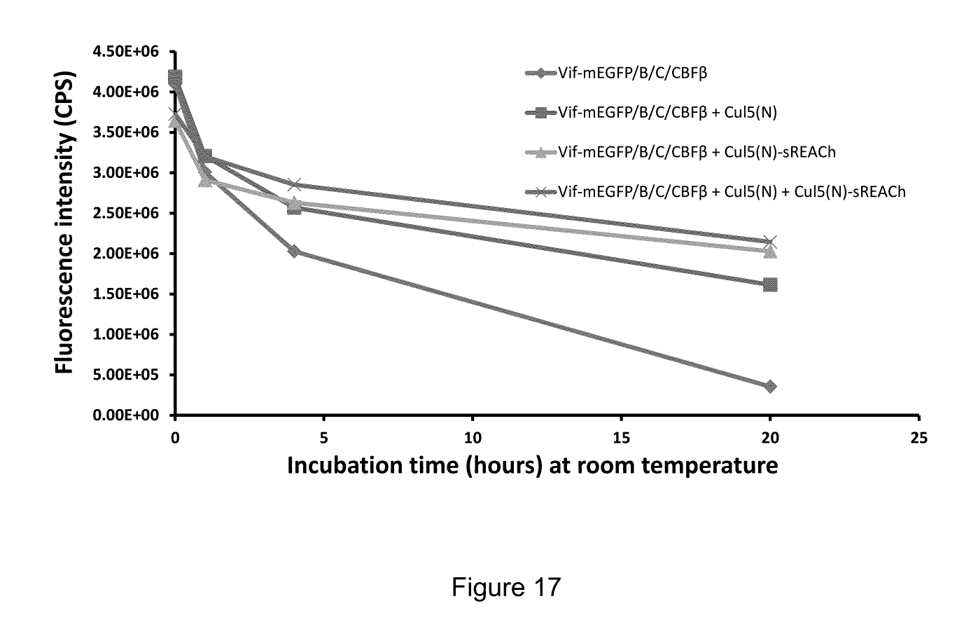

High-throughput experiments on a large scale require that proteins withstand room temperature incubation during sample preparation, liquid handling, and data acquisition. To test the room temperature tolerance of the proteins over an extended screening period, mEGFP tagged quaternary complexes (10 nM) with addition of either 4 molar excess of Cul5(N), Cul5(N)-sREACh (quencher), or both were incubated at room temperature for 20 hours, where fluorescence intensity was monitored at various time points shown (FIG. 17). The results show a loss of fluorescence intensity (FIG. 17) for the free and quenched complexes with time suggesting a loss of protein, possibly due to self-association. No appreciable proteolytic cleavage was observed, and protease inhibitors did not alter this effect.

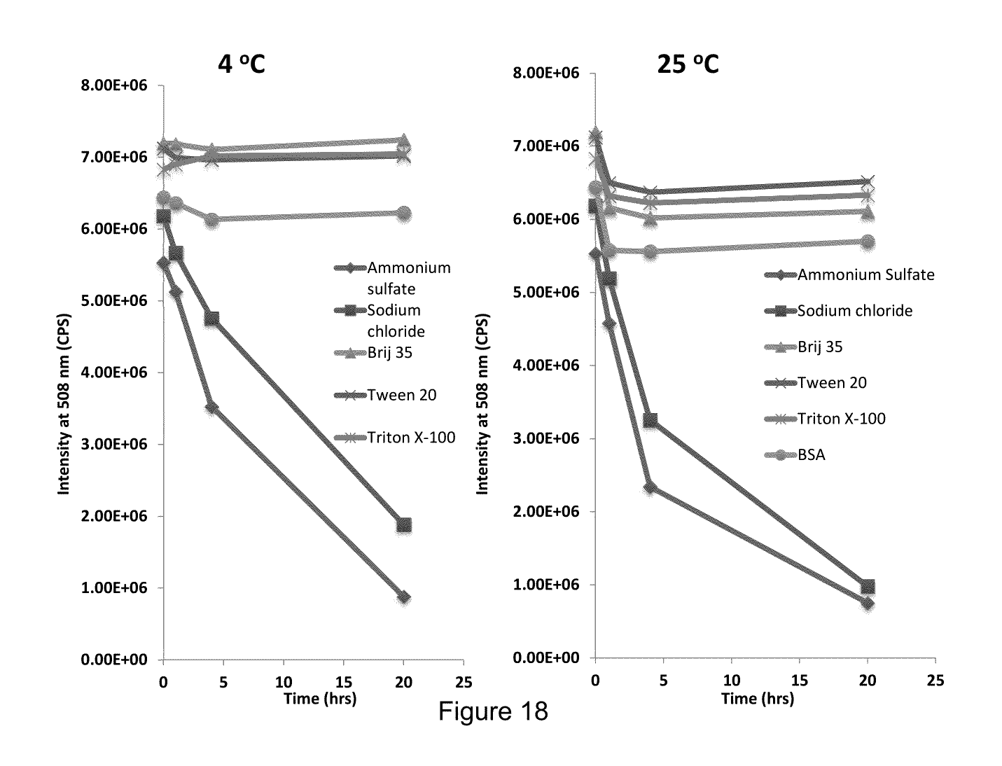

Screens were conducted of agents perceived to block protein aggregation. Kosmotropic salts such as (NH₄)₂SO₄ were tested, as well as variety of non-ionic detergents, which have shown success in membrane protein solubilization. In addition, the protein BSA was also used since it is known to block non-specific binding interactions. The best success was achieved by the detergents and BSA. Due to its low cost and stability, Brij 35 was chosen to be used, which has clear efficacy in maintaining high levels of fluorescence as a function of time at 4° and 25° C. (FIG. 18).

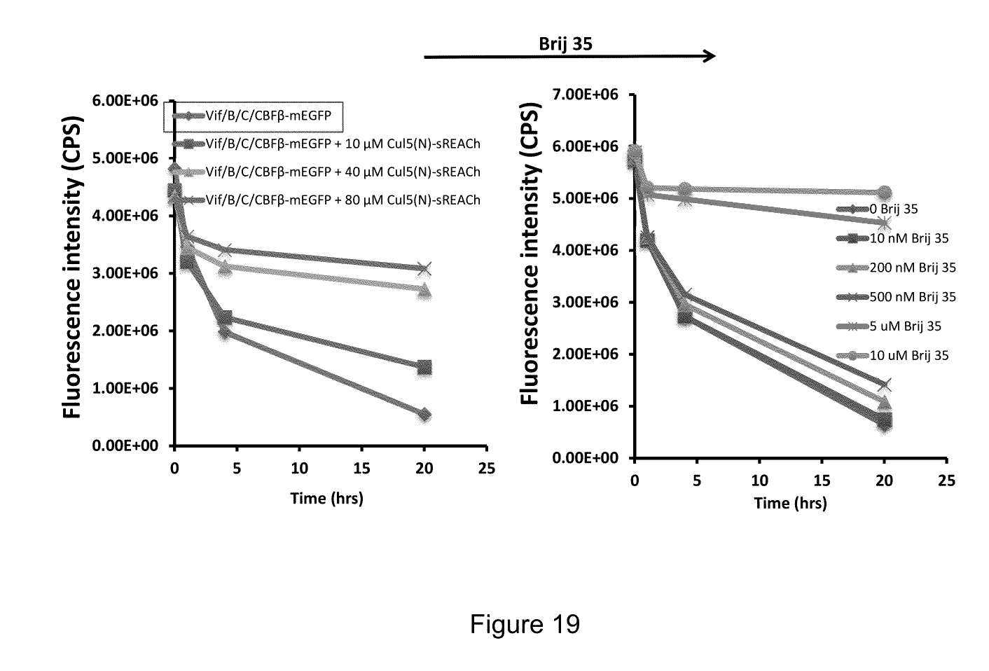

Experiments were conducted to identify the amount of Brij 35 that was necessary and sufficient to stabilize fluorescence as a function of time. Brij 35 proved useful at concentrations significantly less than other proteins, and could also be used in protein purification. A concentration of 10 μM Brij 35 was found to be sufficient for stabilization. FIG. 19 depicts the comparison between non-detergent-containing assay proteins (left) to those with various amounts of Brij 35. The Vif/B/C/CBFβ-mEGFP complex (box) is shown (right) in the presence of varying concentrations of Brij 35. The value of 10 μM is significantly less than the Brij 35 CMC, which is 90 μM. The results shown are representative of all assay complex combinations.

It was determined that the stability of the screening solution requires the use of the detergent Brij 35 as well as 4% glycerol. The optimal FqRET assay buffer used for our benchmarks was: 125 mM NaCl, 50 mM HEPES pH 7.4, 0.2 mM TCEP, 10 μM Brij 35 and 4% glycerol (v/v).

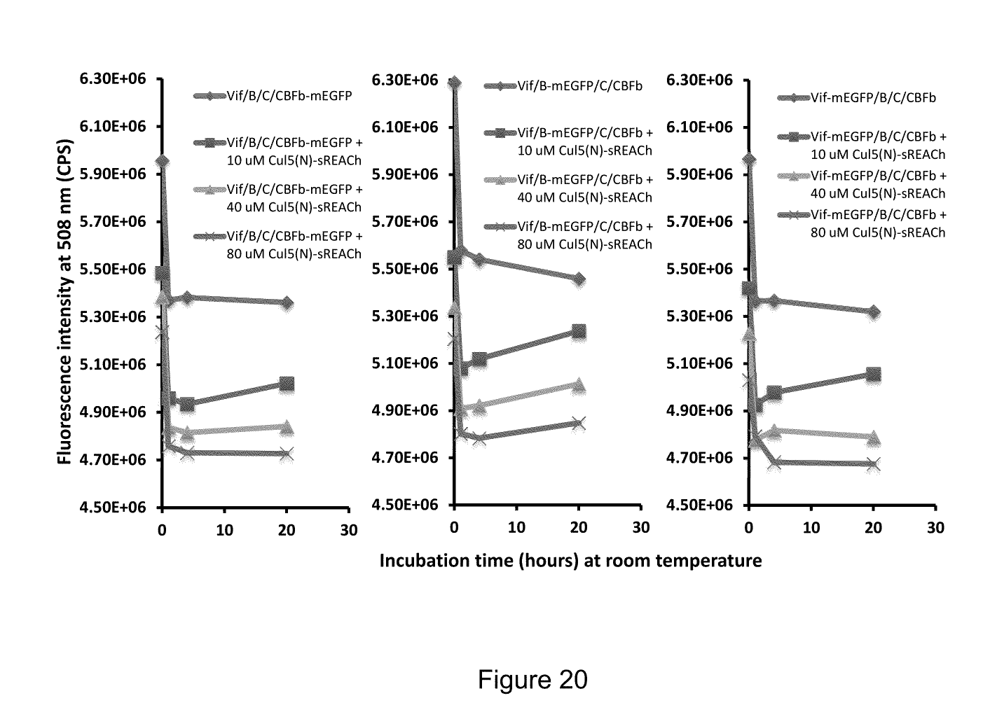

FRET quench testing for the various constructs was performed by titrating different concentrations of C-terminally tagged Cul5(N)-sREACh into 10 μM of mEGFP tagged quaternary complexes. With equimolar concentration of quencher about 8% decrease in fluorescence was observed on average (FIG. 20). Excitation and emission wavelengths that were used were 469 and 508, respectively.

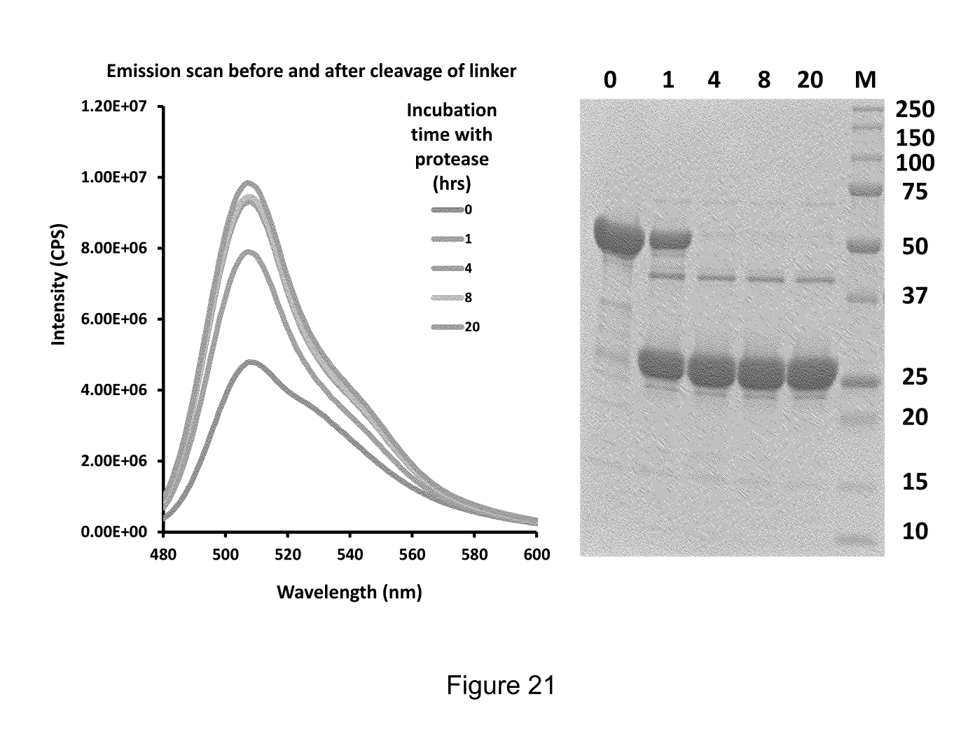

To test for maximum quench capacity, a fusion protein was constructed comprising the two fluorescent proteins of the assay, mEGFP and sREACh, separated by a short flexible linker, which is cleavable by a specific protease. This experiment was done to utilize the known Förster radius of the FRET pair in the context of an optimal linker to achieve an appreciable quench. Once proteins were cleaved, about 50% increase in fluorescence was observed after 20 hours of TEV protease digestion (FIG. 21), which provided information on the appropriate linker for the assay.

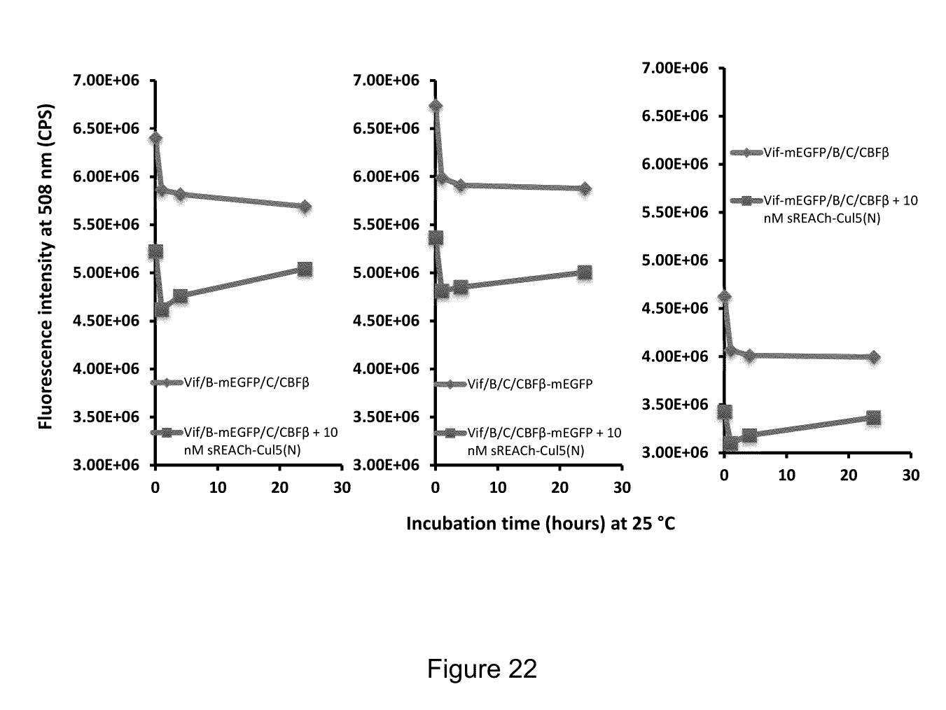

The experiments using the positive control suggested that chromophores in the previous constructs were either too far apart, or conformationally restricted such that they prevented an efficient quench (i.e. low signal-to-noise). To alleviate this problem, the quencher protein (sREACh) was re-engineered to be located on the N-terminus of Cul5(N) rather than its C-terminus. Meanwhile the flexible linker from the mEGFP-sREACh positive control construct was utilized to join the proteins in the configuration sREACh-linker-Cul5(N). On average 20% quenching was observed and the complex appears stable over 24 hr at 25° C. (FIG. 22).

The benchmarks for quenching are as follows. The Vif quaternary complex, where Vif protein is tagged with mEGFP has the best quenching potential (Table 5) with sREACh-Cul5(N) and is used as a primary screen in high-throughput development. Quaternary complexes, where EloB or CBFβ are tagged with mEGFP will be used as additional screens with similar capabilities. This data demonstrates that the quenching efficiency gives a high signal-to-noise in the screening assay.

TABLE 5
Quenching potential of various tagged quaternary complex
upon incubation with sREACh-Cul5(N).

| | Incubation time (hours) | | | |
|---|---|---|---|---|
| | at room temperature | | | |
| Protein sample | 0 | 1 | 4 | 24 |
| Vif/B-mEGFP/C/CBFβ | 18.40% | 21.24% | 18.17% | 11.41% |
| Vif/B/C/CBFβ-mEGFP | 20.36% | 19.65% | 17.95% | 14.84% |
| Vif-mEGFP/B/C/CBFβ | 25.89% | 23.75% | 20.71% | 15.74% |

### Example 6

### Sequences

The amino acid sequences and nucleic acid sequences of components of the screen of the present invention, as described elsewhere herein, include those listed in Table 6 below.

TABLE 6
Exemplary sequences

| | |
|---|---|
| Human EloB | SEQ ID NO: 23 |
| Human EloC | SEQ ID NO: 24 |
| HIV-1 HXB2 Vif(95-192) | SEQ ID NO: 25 |
| HIV-1 Full-length HXB2 Vif (1-192) | SEQ ID NO: 26 |
| Human CBFβ Isoform 2 | SEQ ID NO: 27 |
| GST-Cul5(N)(human residues 2-384, with V341R, L345D | SEQ ID NO: 28 |
| mutations) (Note: 3C cleavage site: QG) | |
| Cul5(N) (post 3C cleavage) | SEQ ID NO: 29 |
| EloB(human residues 1-118)-mEGFP (with A206K mutation) - DNA | SEQ ID NO: 30 |
| sequence | |
| EloB(human residues 1-118)-mEGFP (with A206K mutation) -Peptide | SEQ ID NO: 31 |
| sequence | |
| CBFβ(human residues 1-182)-mEGFP(with A206K mutation) - DNA | SEQ ID NO: 32 |
| sequence | |
| CBFβ(human residues 1-182)-mEGFP(with A206K mutation) - | SEQ ID NO: 33 |
| Peptide sequence | |
| HIV-1 HXB2 Vif(1-192)-mEGFP(with A206K mutation) - DNA | SEQ ID NO: 34 |
| sequence | |
| HIV-1 HXB2 Vif(1-192)-mEGFP(with A206K mutation) - Peptide | SEQ ID NO: 35 |
| sequence | |
| GST-Cul5(N)(human residues 2-384, with V341R, L345D mutations)- | SEQ ID NO: 36 |
| sREACh(with F46L, Q69M, F223R mutations) - DNA sequence | |
| GST-Cul5(N)(human residues 2-384, with V341R, L345D mutations)- | SEQ ID NO: 37 |
| sREACh(with F46L, Q69M, F223R mutations) - Peptide sequence | |
| sREACh(with F46L, Q69M, F223R mutations)- Cul5(N)(human | SEQ ID NO: 38 |
| residues 2-384, with V341R, L345D mutations) - DNA sequence | |
| sREACh(with F46L, Q69M, F223R mutations)- Cul5(N)(human | SEQ ID NO: 39 |
| residues 2-384, with V341R, L345D mutations) - Peptide sequence | |
| sREACh(with F46L, Q69M, F223R mutations)-mEGFP(with A206K | SEQ ID NO: 40 |
| mutation) with optimized quench linker - DNA sequence | |
| sREACh(with F46L, Q69M, F223R mutations)-mEGFP(with A206K | SEQ ID NO: 41 |
| mutation) with optimized quench linker - Peptide sequence | |
| 3C protease linker (derived from human Rhinovirus) - DNA sequence | SEQ ID NO: 42 |
| 3C protease linker (derived from human Rhinovirus) - Peptide | SEQ ID NO: 43 |
| sequence | |

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of producing soluble, functionally active, full-length HIV-1 Vif, said method comprising providing to a cell exogenous ElonginB, ElonginC, Vif, and CBFβ polynucleotide; expressing exogenous ElonginB, ElonginC, Vif, and CBFβ polypeptide therefrom; and isolating an ElonginB/ElonginC/Vif/CBFβ tetramer complex from said cell.

2. An isolated protein complex comprising ElonginB, ElonginC, full-length Vif, and CBFβ wherein said full-length Vif is able to bind Cullin5 at benchmarked levels.

3. An isolated protein complex of claim 2, further comprising Cullin5.

4. The isolated protein complex of claim 2, wherein said isolated protein complex comprises amino acids 1-118 of said ElonginB, and amino acids 17-112 of ElonginC.

5. A method of identifying a compound that inhibits the interaction between Vif and Cullin5, said method comprising
providing a mixture comprising Cullin5, a protein complex comprising ElonginB, ElonginC, Vif, and CBFβ, and a test compound under conditions that are effective for binding of Vif to Cullin5; and
detecting whether or not the test compound inhibits binding of Vif to Cullin5, thereby identifying a compound that inhibits the interaction between Vif and Cullin5.

6. The method of claim 5, wherein the test compound that inhibits the binding between Vif and Cullin5 is an inhibitor of lentiviral infectivity.

7. The method of claim 5, wherein said method is a high throughput method.

8. The method of claim 7, wherein said high throughput method is Förster quenched resonance energy transfer (FqRET).

9. The method of claim 8, wherein at least one of ElonginB, ElonginC, Vif, and CBFβ is labeled with a FRET donor and Cullin5 is labeled with a FRET quencher.

10. The method of claim 8, wherein detecting whether or not the test compound inhibits binding of Vif to Cullin5 comprises detecting an increase in fluorescence compared to a condition where the test compound is absent.

11. The method of claim 5, wherein the mixture comprises Brij 35 and glycerol.

12. The method of claim 5, wherein the mixture is formed by providing a first mixture comprising the protein complex and Cullin5, and contacting the first mixture with the test compound.

13. The method of claim 5, wherein the mixture is formed by providing a first mixture comprising the protein complex, contacting the first mixture with the test compound to produce a second mixture, and contacting the second mixture with Cullin5.

14. The method of claim 5, wherein the mixture is formed by providing a first mixture comprising Cullin5, contacting the first mixture with the test compound to produce a second mixture, and contacting the second mixture with the protein complex.

15. A composition that inhibits the binding of full-length Vif to Cullin5.

16. The composition of claim 15, wherein the composition is identified by a screening method comprising the steps of
providing a mixture comprising Cullin5, a protein complex comprising ElonginB, ElonginC, Vif, and CBFβ, and a test compound under conditions that are effective for binding of Vif to Cullin5; and
detecting whether or not the test compound inhibits binding of Vif to Cullin5.

17. The composition of claim 15, wherein the composition comprises at least one amino acid sequence selected from the group consisting of SEQ ID NOs: 1-18.

18. The composition of claim 15, wherein the composition binds to an epitope of Vif, wherein the epitope is defined by at least one amino acid sequence selected from the group consisting of SEQ ID NOs: 1-8.

19. The composition of claim 15, wherein the composition binds to an epitope of Cullin5, wherein the epitope is defined by at least one amino acid sequence selected from the group consisting of SEQ ID NOs: 9-11.

20. The composition of claim 15, wherein the composition reduces the affinity for a Vif-Cullin5 interaction by inhibiting the binding of Vif and CBFβ.

21. A method for inhibiting infectivity of a lentivirus, the method comprising contacting a cell that is producing the virus with an antiviral-effective amount of a composition that inhibits Vif-Cullin5 binding.

22. The method of claim 21, wherein the antiviral-effective amount of the composition does not substantially affect proteins in the cell other than lentivirus Vif.

23. The method of claim 21, wherein the composition is identified by a screening method comprising the steps of
providing a mixture comprising Cullin5, a protein complex comprising ElonginB, ElonginC, Vif, and CBFβ, and a test compound under conditions that are effective for binding of Vif to Cullin5; and
detecting whether or not the test compound inhibits binding of Vif to Cullin5.

24. The method of claim 21, wherein the lentivirus expresses Vif.

25. The method of claim 21, wherein the lentivirus is HIV.

26. The method of claim 21 wherein the composition inhibits the interaction of Vif with cellular Cullin5-E3 ubiquitin ligase, thereby preventing the degradation of the viral inhibitor, APOBEC3G and/or related family members, and thus allowing the APOBEC3G and/or related family members to inhibit viral infectivity.

27. The method of claim 21, wherein the composition comprises at least one amino acid sequence selected from the group consisting of SEQ ID NOs: 1-18.

28. The method of claim 21, wherein the composition binds to an epitope of Vif, wherein the epitope is defined by at least one amino acid sequence selected from the group consisting of SEQ ID NOs: 1-8.

29. The method of claim 21, wherein the composition binds to an epitope of Cullin5, wherein the epitope is defined by at least one amino acid sequence selected from the group consisting of SEQ ID NOs: 9-11.
